(19) United States
(12) Patent Application Publication
(10) Pub. No.: US 2025/0194942 A1
(43) Pub. Date: Jun. 19, 2025

(54) APPARATUS, SYSTEMS, AND METHODS TO IMPROVE ATRIAL FIBRILLATION OUTCOMES INVOLVING THE LEFT ATRIAL APPENDAGE

(71) Applicant: Ablation Innovations, LLC, Brentwood, TN (US)

(72) Inventors: Daniel Walter Kaiser, Nashville, TN (US); Margaret Rox, Brentwood, TN (US)

(21) Appl. No.: 18/935,350

(22) Filed: Nov. 1, 2024

Related U.S. Application Data
(63) Continuation of application No. PCT/US23/20628, filed on May 1, 2023.
(60) Provisional application No. 63/337,135, filed on May 1, 2022.
(60) Provisional application No. 63/444,940, filed on Feb. 11, 2023.
(60) Provisional application No. 63/456,392, filed on Mar. 31, 2023.

(51) Int. Cl. A61B 5/0215; A61B 5/00; A61N 1/37; A61N 1/375; A61N 1/378

(52) U.S. Cl. CPC A61B 5/0215; A61B 5/686; A61B 5/6869; A61B 5/7264; A61B 5/7275; A61B 5/7289; A61B 5/746; A61N 1/3702; A61N 1/3756; A61N 1/378; A61B 2560/0214; A61B 2560/0223; A61B 2560/0462; A61B 2562/0204; A61B 2562/0219; A61B 2562/0247; A61B 2562/0261

(57) ABSTRACT

Apparatus, systems, and methods are provided for an implantable cardiac device designed for placement within or near the left atrial appendage (LAA) of a heart, configured to estimate left atrial pressure (LAP) or changes therein by detecting mechanical changes associated with variations in LAP. The device comprises a cap integrated with one or more mechanical sensors-such as strain gauges, piezoelectric elements, or accelerometers-that detect mechanical deformations corresponding to LAP fluctuations. A processor receives data from these sensors and estimates LAP based on the detected mechanical changes. The device may also include an electrical sensor to measure an electrocardiogram (ECG) signal, allowing the processor to estimate changes in LAP by comparing time delays between the ECG signal and mechanical signals.

### RELATED APPLICATION DATA

The present application is a continuation-in-part of co-pending International Application No. PCT/US2023/020628, filed May 1, 2023, which claims benefit of provisional applications Ser. Nos. 63/337,135, filed May 1, 2022, 63/444,940, filed Feb. 11, 2023, and 63/456,392,filed Mar. 31, 2023, the entire disclosures of which are expressly incorporated by reference herein. The present application is also related to U.S. Publication No. 2020/0269059, the entire disclosure of which is expressly incorporated by reference herein.

### TECHNICAL FIELD

The present application relates to apparatus, systems, and methods for improving atrial fibrillation outcomes involving the left atrial appendage. More specifically, implantable devices are provided that are designed for placement within a patient's body, e.g., within the left atrial appendage of the atrium of a patient's heart to monitor and treat abnormal rhythms.

### BACKGROUND

Atrial fibrillation (“AF”) is the most common sustained cardiac arrhythmia. Cardiac ablation of atrial fibrillation is one of the most common cardiac procedures. The cornerstone of AF ablation procedures has been pulmonary vein isolation (“PVI”). However, in recent years, non-pulmonary vein triggers have been identified. One of the most common non-pulmonary vein triggers is the left atrial appendage (“LAA”). Therefore, there is a growing interest in performing LAA isolation during ablation procedures. However, there are technical difficulties in creating electrical isolation of the LAA. In addition, numerous reports suggest that there is an increased risk of thromboembolic events following LAA isolation. Inadequate function of the LAA after electrical isolation is felt to be responsible. After electrical isolation, the LAA does not squeeze adequately. As a result, blood can coagulate to form a thrombus in the LAA. This thrombus can then embolize to other parts of the body, which can cause a stroke.

The LAA has various morphologies and sizes. An ablation tool needs to be adaptive enough to accommodate these differences. Some LAAs have a straight structure ('windsock' morphology) while other LAA morphologies include a sharp bend ('chicken-wing' morphology). In addition, an ablation tool that prevents thrombus formation within the LAA is optimal. There are data that suggest that some patients who are in sinus rhythm remain at risk for thrombus formation in the LAA after electrical isolation of the LAA.

The only commercially available leadless pacemaker is intended to be placed in the right ventricle. There are next-generation leadless pacemakers under development to enable placement within the right atrium. However, there are no known devices available or in development that describes a leadless pacemaker designed to be placed within the left atrium. Therefore, improved tools to improve AF ablation procedures, e.g., by monitoring AF episodes, delivering anti-tachycardia pacing (“ATP”) pulses, and/or isolating the LAA without the increased risk of thromboembolic events, may be useful.

Monitoring left atrial pressure (LAP) is crucial in managing patients with heart failure and atrial fibrillation (AF). Elevated LAP is a key indicator of worsening heart failure and can precede clinical symptoms by days or weeks. Traditional methods of measuring LAP are invasive and not suitable for continuous monitoring. Non-invasive techniques lack the accuracy needed for effective patient management.

Implantable devices that can estimate LAP offer a promising solution for continuous monitoring. By detecting mechanical changes within the left atrial appendage (LAA), these devices can provide real-time data on LAP fluctuations. Mechanical sensors such as strain gauges, piezoelectric elements, and accelerometers can detect subtle changes in the LAA's structure corresponding to pressure variations.

Integrating electrical sensors to measure electrocardiogram (ECG) signals allows for a comprehensive analysis of cardiac function. By comparing the timing between mechanical signals and ECG signals, the device can more accurately estimate changes in LAP. This correlation is essential for identifying specific phases of the cardiac cycle and understanding the hemodynamic status of the patient.

The ability to store historical LAP data and analyze trends over time enables early detection of worsening heart failure. The device can trigger alerts when LAP exceeds predetermined thresholds, allowing for timely medical interventions. Additionally, incorporating a leadless pacemaker into the device provides therapeutic capabilities, such as delivering pacing pulses and adjusting heart rate to optimize cardiac output and enhance the identification of the patient's volume status.

Given the various morphologies and sizes of the LAA, an adaptive device that can accommodate these differences is essential. A device equipped with a cap integrated with mechanical sensors can conform to different LAA shapes, such as ‘windsock’ or ‘chicken-wing’ morphologies. This adaptability ensures accurate measurements across a wide patient population.

Furthermore, after LAA isolation procedures, there is a risk of thrombus formation due to inadequate contraction of the LAA. An implantable device within the LAA can monitor mechanical activity and ensure that the LAA is functioning properly post-isolation, potentially reducing the risk of thromboembolic events.

Currently, there are no commercially available devices designed to be placed within the left atrium for these purposes. The development of an implantable cardiac device that can monitor LAP, analyze cardiac signals, and provide therapeutic interventions addresses a significant unmet need in cardiac care.

### SUMMARY

The present application relates to apparatus, systems, and methods for estimating left atrial (LA) pressure and predicting cardiac conditions, including heart failure hospitalization and exacerbation, using mechanical and electrical sensor data from the left atrial appendage (LAA). The system may be designed to minimally-invasively estimate LA pressure, analyze time delays between mechanical and electrical cardiac events, and/or identify early signs of heart failure exacerbation, which may ultimately improve patient management and clinical outcomes. The device and methods herein may also relate to monitoring AF episodes, delivering ATP pulses, and/or achieving electrical isolation of the left atrial appendage (LAA) of a patient's heart and/or preventing thrombus formation after electrical isolation.

More particularly, the systems and methods herein may include a device that is implanted from within the left atrium, isolates the LAA, and prevents thrombus formation within the LAA. In addition, the device may include material that facilitates endothelialization. In one example, the device may be placed within the body through a sheath and takes a desired shape after leaving the deployment sheath. The device may also include systems and methods for measuring LA pressure, providing ATP pulses, monitoring AF, and/or providing electrocautery to seal the LAA, prevent microbleeds, and securely anchor the device.

In one example, there are three primary portions of the device, including an anchoring portion, a coupling portion, and a covering portion. The device may include one or more of these components.

The anchoring portion is deployed first and anchored with the left atrial appendage. In some examples, the anchoring portion undergoes a conformational change within the left atrial or left ventricular, and then is advanced into the left atrial appendage for anchoring. The anchoring portion can adhere to the left atrial appendage through a variety of methods, but primarily through radial forces and tines. In some circumstances, a screw mechanism may attach the anchoring portion to the left atrial appendage tissue. In some examples, the anchoring portion can adhere to the left atrial appendage through electrocautery or some other delivery of energy that causes the tissue to attach to the tines. After re-arranging the shape within the left atrial or left ventricle, the anchoring portion may be collapsed to enable entry into the left atrial appendage. Once optimally positioned within the left atrial appendage, the device is enlarged. The anchoring portion may be enlarged by releasing a tether holding the portion in a collapsed form. In other examples, the anchoring portion has an actuator that adheres the anchor to the left atrial appendage. In some examples, the anchoring portion includes a plurality of elongated subunits connected by connectors. The connections may be biased in a certain manner to either collapse or enlarge the anchoring portion. In other examples, the connectors permit certain rotations relative to other subunits to anchor the device within the left atrial appendage. In some examples, the connectors move the subunits through electrical current, heat, electrical motors, or a screw mechanism. Various methods to release a tether or tethers may enable each connector to activate in a pre-specified manner. In other examples, the activation of electrical motors within various connectors enables the device to enlarge and adhere to the left atrial appendage in a controlled manner.

In another example, some or all the connectors between the various elongated subunits may include an electrical motor. The electrical motor may be a hinged motor or servo motor. The hinged motor can be used to collapse or enlarge the anchoring portion as needed to facilitate entry into the left atrial appendage and/or enlarge the device to anchor the device within the left atrial appendage. Various sensors along the device can be used to measure contact with tissue within the heart. In some examples, changes in impedance can be used to estimate tissue contact and/or force. In other examples, changes in resistance, optical properties, and/or deflection may be measured to determine contact force.

The anchoring portion can be advanced into the left atrial appendage in a variety of mechanisms. In some examples, an intracardiac echocardiography (ICE) catheter is advanced through the device and into the left atrial appendage. In some examples, the ICE catheter is surrounded by a balloon to prevent excessive force against cardiac tissue. The balloon may also contain a pressure sensor to measure the force the balloon and/or ICE catheter is pressing against tissue. In other examples, the ICE catheter has cushioning around the tip to prevent excessive force and/or perforation of cardiac structures. The ICE catheter may be advanced through the device and guided into the left atrial appendage. The ICE catheter may be used to outline the walls of the left atrial appendage to create a detailed map of the left atrium and the left atrial appendage. This electroanatomical map may be used to deploy the device into the left atrial appendage.

In another example, a leadless pacemaker device is provided for implantation within or near a left atrial appendage extending from a left atrium of a heart to monitor and/or treat a patient with conduction abnormalities and/or cardiac dysrhythmias. For example, the device may include a battery capable of storing electrical energy; an anchoring portion to anchor the device within the left atrial appendage; at least one electrode configured for sensing and pacing the left atrium; a control processor configured for processing data; a communication module to communicate to other devices; and a cover portion configured to prevent thrombus from within the left atrial appendage of the left atrium to embolize out of the left atrial appendage of the left atrium. In one example, the cover portion is configured to be rotated independently of the anchoring portion.

In some examples, a catheter is advanced through the device into the left atrial appendage. This catheter may function as a rail to guide the device into the left atrial appendage. In other examples, the delivery sheath may be adjusted to direct the device into the left atrial appendage. In other examples, the electroanatomical map may guide adjustment to delivery sheaths or the device through mechanical or motorized methods to guide the device into the left atrial appendage. In certain examples, the device is advanced with no additional catheters but includes pressure sensing elements to prevent perforation. The device may have a spring mechanism, e.g., between the covering portion and the anchoring portion with the ability to sense force (e.g., via deflection, resistance, or impedance detection, optical fibers, or magnetic sensing).

The coupling portion couples the anchor portion to the covering portion. In some examples, the coupling portion adjusts the distance between the anchor portion and the cover portion. The coupling portion may also measure the amount of force between the coupling portion and the anchoring portion, the amount of force being used against the tissue, or changes in left atrial pressures. The distance between the anchor and cover may be adjustable in a variety of methods. In one example, a screw is turned which pulls a thread to bring the components together. In another example, a screw advances along a following mechanism to advance or retract the covering portion with respect to the anchoring device. An electrical motor may be used to adjust the distance between the cover and the anchor. In another example, a mechanical arm is adjusted which approximates the two structures. The coupling portion may be adjusted to bring the cover against the ostium of the left atrial appendage. The cover may be positioned in the appropriate location within the left atrium such that as the coupling portion approximates the cover towards the anchor, the covering portion covers all aspects of the left atrial appendage.

The cover portion is designed to prevent thrombus from within the left atrial appendage of the left atrium to embolize out of the left atrial appendage of the left atrium. The cover portion may be a substantially flat circle, or may have an elliptical or other non-circular shape. In some examples, the cover portion has a predesigned shape to fit optimally against the opening of the left atrial appendage. The cover portion may have sensors to detect left atrial pressures.

Additionally, the cover portion may be used to capture the LV or LA for pacing. Capturing may take place inside the left atrial appendage either distally or adjacent to the LV using the anchoring device. Capturing may also take place outside the LAA using the cover portion. The pacing electrode may be positioned 0 to 2 centimeters away from the ostium of the left atrial appendage.

In some examples, a pacing system may be provided that includes a pacemaker battery and/or processor housed within a Nitinol frame. In these examples, at least part of the pacemaker housing includes portions of an elongated tube of Nitinol. The Nitinol tube may be cut at spaced-apart distances, e.g., to provide precise cut-outs, to enable the Nitinol tube to fold into a desired shape. This folded Nitinol tube may then be heat-set and/or otherwise treated so that the elongated tube is configured to translate into a pre-designed shape, such as those described elsewhere herein, when deployed and/or otherwise able to move freely out of a delivery sheath. In this manner, a single nitinol tube provides adequate strength to contain the pacemaker components while also enabling the device to fold into a precise shape to enable advancement and deployment into the left atrial appendage. The cuts into the Nitinol may utilize similar technologies well known in the cardiac stent and cardiac valve technologies. In other examples, Nitinol wire may be braided into a desired shape. When utilizing a Nitinol tube, various cuts into the tube can facilitate the Nitinol tube to later be folded, rotated, curved, extended, collapsed, bent, revolved, spun, twirled, translated, or pivoted into the desired position.

The Nitinol may be shape-set into the desired shape via various mechanisms and techniques. For example, the Nitinol wire or tube may be placed into the desired shape and then heat-treated at a closely controlled time and temperature. In addition to or alternatively, electrical heating may also be utilized. Various other solutions may also be utilized to facilitate the process.

In another example, a pacing device may be biased to assume a desired shape as the device is advanced out of a delivery sheath. Alternatively, actuators or other mechanisms may be provided to allow or cause the device to bend, rotate, or otherwise translate into the desired shape other than merely being advanced out of a delivery sheath. For example, the device may include a wire adjacent to the pacemaker housing that maintains a desired shape, e.g., a generally straightened or other delivery configuration. Once the wire is withdrawn, the device is then free to bend, rotate, or otherwise translate into the desired shape. In other examples, tiny motors or forces may be utilized to control the movement of subunits of the device. In other examples, magnetic forces may be used.

In still another example, a plurality of battery subunits of an LAA device may automatically fold or spiral or are able to collapse into a more dense structure. In one example, an external self-expanding cage-like structure is anchored into the LAA. Then, the cage is filled with battery subunits. Finally, a cap or covering portion is placed on the outside of the LAA, similar to other systems herein. In another example, an expanding cage is anchored into the LAA that has a cap over the front of the LAA. Battery subunits are then advanced into the LAA through a small opening in the cap.

In another example, an anchoring mechanism may be deployed into the LAA. Next, the battery and/or processor subunits may be deployed inside of the anchoring mechanism. In some examples, the cap may be placed after the batteries and/or processor are deployed. In another example, the anchor and cap are deployed first. Then, the batteries and/or processor are deployed through the cap into the LAA space with the anchoring mechanism maintaining the device in place.

In another example, a pacemaker device, such as any of those described herein, implanted in the left atrial appendage may communicate with other pacemakers, e.g., implanted elsewhere in the patient's body. For example, a leadless ventricular pacemaker can be placed into the left and/or right ventricle. The LAA pacemaker may communicate or synchronize the timing of pacing through a variety of mechanisms. The two devices may operate independently but be programmed to similar signals to function in a coordinated way. This may include one of these devices (or yet another implanted device) being configured to send communication via pacing below the capture threshold or pacing at times the myocardium is unlikely to capture. In another example, numerous devices may respond to physiologically sensed episodes to coordinate function.

Because Bluetooth capabilities may drain battery life, optionally, any of the LAA devices herein may communicate through other modalities to other, more readily accessed areas inside or outside the body to communicate via Bluetooth or other wireless communication. For example, an LAA pacemaker device may communicate with a typical dual-chamber pacemaker. The LAA device may deliver pacing coordinating timing with the traditional pacemaker to synchronize the two atria. The LAA device may communicate wirelessly with the traditional pacemaker through low-energy communication, while the traditional pacemaker may communicate this information outside the body using Bluetooth or other wireless communication protocols.

In another example, the typical pacing stimulations may include characteristics that enable the LAA pacemaker devices herein to understand the timing of function of the other devices. In one example, the pacing stimulus from the atrium may include characteristics that can be sensed by the LAA device to understand or differentiate device functioning.

In some examples, measurements of the left atrial appendage are obtained using a preprocedural CT scan. In other examples, a trans-esophageal echocardiogram or intracardiac echocardiogram (ICE) imaging is used to measure the left atrial appendage and guide device implantation. In some examples, TEE imaging is performed prior to the implantation procedure. In this example, measurements of the left atrium and left atrial appendage are made using the TEE images. These measurements can then be incorporated into a three-dimensional map to help guide and deploy the device. The TEE images can be used to measure the LAA dimensions and plan how to deploy the device. The TEE images can be made with a TEE probe which is then removed. However, the 3D images can still be used to guide the implantation procedure. The device can use sensors correlating to pressure to determine if the device is appropriately anchored into the LAA. The sensors can also make sure the pressure is not too high, which could lead to complications. In addition, other sensor data, including positioning, impedance, accelerometer, and oxygen sensors can be used to guide implantation or to help with device function.

In some examples, TEE imaging can also assist in performing ablations. For example, ablation or electroporation energy can be delivered from the device. TEE imaging can create left atrial anatomy to determine the location of pulmonary veins and the interatrial septum. This data can be incorporated into 3D mapping to guide aspects of the procedure such as transseptal puncture, guiding ablation tools to the pulmonary veins or left atrial appendage, and implanting the device in the left atrial appendage. The cap covering the ostium of the left atrial appendage may use a pressure sensor or impedance data to verify the left atrial appendage is completely closed.

In one example, a system may be provided that includes an implantable device configured to be positioned within the LAA, equipped with sensors to detect mechanical deformation, vibrations, or other characteristics of the LAA in response to cardiac activity. The device may also integrate an electrocardiogram (ECG) module to provide electrical signals corresponding to cardiac electrical activity. The system may use a combination of strain gauges, accelerometers, pressure sensors, or other mechanical sensors to collect data from the LAA. A data acquisition system collects and synchronizes the mechanical and electrical signals, while a processing unit analyzes the collected data, estimates LA pressure, and predicts patient outcomes based on physiological markers. Machine learning algorithms may also be used to refine the estimation and prediction models.

The device may include a cap that covers at least a portion of the apparatus when implanted within or near the left atrial appendage (LAA) of the heart. The cap may integrate one or more mechanical sensors, such as strain gauges or piezoelectric elements, configured to detect mechanical changes associated with variations in left atrial pressure (LAP). These sensors may measure mechanical deformations of the cap caused by pressure fluctuations within the LAA, allowing for continuous and accurate estimation of LAP.

A processor within the device may receive data from the mechanical sensors and utilize algorithms to estimate LAP or changes in LAP based on the detected mechanical changes. The processor may perform waveform analysis to identify and analyze components of the atrial pressure waveform, including the a wave, c wave, and v wave. By analyzing changes in the timing and amplitude of these mechanical signals, the processor may determine the patient's volume status and detect early signs of worsening heart failure.

The device may also include at least one electrical sensor configured to measure the patient's electrocardiogram (ECG) signal. By comparing time delays between the ECG signal and the mechanical signals from the mechanical sensors, the processor may more accurately estimate changes in LAP. This correlation enables the alignment of mechanical measurements with specific phases of the cardiac cycle, enhancing the precision of LAP estimations.

To provide a holistic view of cardiac function, the device may further include accelerometers to detect motion or vibrations associated with cardiac activity, offering additional data points for LAP estimation. A microphone may capture heart sounds for diagnostic purposes, aiding in the detection of murmurs or other cardiac anomalies. Pressure sensors integrated into the device may provide periodic calibration, ensuring long-term accuracy of LAP measurements by cross-referencing mechanical sensor data with real pressure readings.

In another example, the device may include biochemical sensors to assess left atrial pressure (LAP) through the detection of cardiac stress biomarkers. A chemically responsive membrane, embedded within the device, can measure levels of specific biochemicals such as natriuretic peptides or nitric oxide that correlate with increased atrial pressure due to hemodynamic stress. These chemical concentrations can be analyzed by an integrated processor to infer fluctuations in LAP without reliance on physical deformation sensors. This method allows for continuous, real-time biochemical monitoring within the atrium, providing an innovative alternative to mechanical LAP estimation.

The device may further include an ultrasonic Doppler sensor or optical flow monitor to assess left atrial pressure (LAP) indirectly by analyzing blood flow velocities within the left atrium or pulmonary veins. By measuring changes in flow dynamics correlated with LAP fluctuations, the device can calculate LAP levels without directly measuring pressure within the LAA. An integrated processor can use flow velocity data to model LAP changes, providing a non-invasive, indirect method of estimating LAP. This approach offers continuous LAP monitoring by leveraging flow characteristics rather than relying on strain-based or deformation-based measurements.

The processor may be configured to store historical data of LAP measurements, allowing for long-term trend analysis. By analyzing these trends over time, the device may detect early signs of worsening heart failure or other cardiac conditions. Optionally, the processor may adjust device operation accordingly to optimize patient outcomes and trigger alerts when estimated LAP exceeds predetermined thresholds, enabling timely medical interventions.

In one example, systemic blood pressure may be estimated using waveform analysis and pulse transit time (PTT), leveraging signals detected within the heart and correlating them with downstream vascular pulses. This method involves measuring the time delay between the onset of a pressure wave in the left atrium or left ventricle (LV) and the arrival of this pulse in peripheral arterial vessels. This delay, or transit time, inversely correlates with blood pressure; higher systemic blood pressure results in a faster pulse wave velocity, reducing PTT. By continuously monitoring these transit times and correlating them with baseline data, the device can estimate variations in systemic blood pressure indirectly.

Another example involves measuring pulse wave velocity (PWV), which is strongly associated with blood pressure changes. Using a Doppler or acoustic sensor within the device, the speed of the blood pulse wave traveling from the LV or atrium can be measured. Since systemic blood pressure affects arterial stiffness, leading to an increase in PWV when blood pressure is elevated, tracking PWV trends can provide a non-invasive way to infer systemic blood pressure indirectly. This approach allows the device to assess the rate at which the pulse wave propagates from the heart to the vasculature, indicating shifts in blood pressure over time without requiring a direct arterial blood pressure measurement.

In another example, left atrial pressure (LAP) measurements may be used as a proxy for systemic blood pressure. Although LAP itself does not directly reflect systemic blood pressure, it may provide valuable insights under specific conditions, especially in patients with heart failure or volume overload, where LAP correlates with overall hemodynamic status. By combining real-time LAP data with signals from the LV, such as stroke volume or contractility (detected through pressure or accelerometer-based sensors), a more comprehensive picture of blood pressure trends may be estimated. This technique may use algorithms to interpret changes in LAP alongside LV performance, identifying potential systemic blood pressure shifts based on intracardiac pressures and ventricular response.

Optionally, optical or photoacoustic sensors within the device may estimate blood pressure by detecting hemoglobin concentration changes and vascular dynamics in adjacent tissues. By emitting light pulses, these sensors measure the expansion and contraction of arterial vessels around the atrium or ventricle. The variations in vessel diameter over time can reflect changes in systemic blood pressure, particularly if the sensor is positioned to detect pulsatile changes in arterial cross-sectional area. This optical method provides a minimally invasive way to monitor blood pressure trends and vascular health continuously.

In some examples, a machine learning algorithm may integrate data from these different sensors to provide a real-time systemic blood pressure estimate. By analyzing patterns in LAP, PTT, PWV, and stroke volume metrics, the algorithm may adapt to each patient's baseline measurements, learning to identify subtle changes that correlate with shifts in systemic blood pressure. This approach allows for a continuous, personalized monitoring of blood pressure, leveraging multiple physiological indicators to enhance accuracy without additional invasive sensors.

In some examples, the device is equipped with a communication component, which may transmit data to an external receiver or clinician's monitor. This feature may facilitate remote monitoring and management of the patient's cardiac health, allowing healthcare providers to make informed decisions based on real-time data. A power source may supply energy to the device, supporting its continuous operation. Energy-efficient designs and low-power components may extend battery life, reducing the need for frequent replacements or interventions.

The device may integrate a leadless pacemaker configured to deliver pacing pulses directly to the left atrium. This pacemaker may adjust the heart rate to enhance the identification of the patient's volume status and improve atrial synchrony, particularly in patients with delayed interatrial conduction. By delivering anti-tachycardia pacing (ATP) pulses, the device may help manage atrial fibrillation episodes effectively.

Given the diverse morphologies and sizes of the LAA, such as “windsock” or “chicken-wing” shapes, the device may be designed to be adaptive. The cap and anchoring mechanisms may conform to different anatomical structures, ensuring secure placement and optimal sensor function across a wide patient population.

The cap may not only house mechanical sensors but also serve as a barrier to prevent thrombus formation within the LAA from embolizing to other parts of the body. By effectively sealing the LAA opening, the device may reduce the risk of stroke associated with thromboembolic events following LAA isolation procedures.

The anchoring portion may be deployed within the LAA, utilizing radial forces, tines, or screw mechanisms to secure the device. It may also employ electrocautery or energy delivery to adhere to LAA tissue. The coupling portion may adjust the distance between the anchoring portion and the cap, allowing for optimal positioning and force application against the LAA ostium. The covering portion (cap) may be designed to fit the LAA opening precisely and may have sensors for pressure detection and electrodes for pacing. The cap may ensure complete closure of the LAA to prevent thrombus escape.

Methods for precise deployment and positioning of the device may be incorporated, such as the use of intracardiac echocardiography (ICE) catheters with cushioning or balloon mechanisms to prevent tissue damage during deployment. Creation of detailed electroanatomical maps using ICE or transesophageal echocardiography (TEE) imaging may guide device placement. Sensors on the device may measure contact force, impedance, and tissue interaction during deployment.

Optionally, the device may communicate and synchronize with other implanted cardiac devices, such as ventricular pacemakers, to coordinate cardiac function. Communication may occur through low-energy wireless protocols or through intrinsic cardiac signals, enhancing overall cardiac therapy effectiveness. Machine learning algorithms may be used in the processor to refine LAP estimation and patient outcome predictions. These algorithms may learn from historical data to improve accuracy over time, offering personalized monitoring and treatment adjustments.

The device may enable continuous, minimally invasive monitoring of LAP, a critical parameter in managing heart failure and atrial fibrillation. For example, the device may provide early detection of cardiac decompensation, allowing for proactive interventions. The device may reduce the need for invasive procedures to measure LAP and enhance patient management by providing real-time data and alerts to clinicians.

The systems and methods herein may have applications in substantially continuous cardiac monitoring for patients with conditions such as heart failure or atrial fibrillation, enabling non-invasive or minimally invasive estimation of LA pressure. For example, the systems may enable clinicians to monitor patients' cardiac conditions remotely, providing real-time data to facilitate proactive management and reduce hospitalizations. The system may also be used to optimize pacing in patients with delayed interatrial conduction to improve atrial synchrony.

Other aspects and features including the need for and use of the present invention will become apparent from consideration of the following description taken in conjunction with the accompanying drawings.

### BRIEF DESCRIPTION OF THE DRAWINGS

FIG. 1 is a simplified functional block diagram of an example of a leadless pacemaker device.

FIGS. 2A and 2B are cross-sectional views of an example of a leadless pacemaker undergoing a first conformational change within the left atrium.

FIG. 3 is a schematic illustration of an example of a leadless pacemaker in a contracted state within the left atrium ready for advancement into the left atrial appendage.

FIG. 4 is a schematic illustration of the leadless pacemaker demonstrated in FIG. 3 with the device undergoing a second conformational change as a method to anchor the device within the left atrial appendage.

FIG. 5 is schematic illustration of an example of the leadless pacer being advanced into the left atrial appendage.

FIGS. 6A and 6B are schematic illustrations of a leadless pacemaker entering and then anchoring within the left atrial appendage.

FIG. 7 is a side view of an example of a leadless pacemaker device designed for implantation within the left atrial appendage that includes three portions (a cover portion, a coupling portion, and an anchoring portion) deployed from a distal portion of a delivery sheath.

FIG. 8 is a schematic illustration of an example of the leadless pacemaker device with the cover portion with pacing electrodes being advanced to occlude the left atrial appendage ostium.

FIG. 9 is a flow diagram of an exemplary method for deploying a leadless pacemaker device.

FIG. 10 is a flow diagram of another exemplary method for deploying a leadless pacemaker device.

FIG. 11 is a view of an example of a leadless pacemaker undergoing a first conformational change.

FIG. 12 is a cross-sectional view of the leadless pacemaker of FIG. 11 being deployed inside the left atrial appendage.

FIG. 13 is a schematic illustration of an example of the leadless pacemaker device with a cover portion.

FIGS. 14A and 14B are cross-sectional views of a left atrial appendage showing the leadless pacemaker of FIG. 13 being deployed inside the left atrial appendage after the anchor has been deployed.

FIG. 15 shows another example of a leadless pacemaker.

FIGS. 16A-16C show another example of a pacing device including a Nitinol tube cut to provide a device that folds upon itself when deployed.

FIGS. 17A-17C show another example of a Nitinol tube that may be used for a pacing device introduced into a delivery sheath.

FIGS. 18A-18D show another example of a pacing device including a Nitinol tube carrying subunits that are in a deployed or folded configuration.

FIGS. 19A-19D show still another example of a pacing device including a Nitinol tube carrying subunits that are in a deployed or folded configuration.

FIGS. 20A and 20B show the device of FIGS. 19A-19D being deployed within a left atrial appendage.

FIGS. 21A-21C show yet another example of a pacing device including a Nitinol tube carrying subunits that are in a deployed or folded configuration.

FIG. 22 shows another example of a pacing device in a deployed configuration.

FIG. 23 shows another example of a pacing device in a deployed configuration.

FIG. 24 is a cross-section of a heart showing potential capture locations for pacing from the LAA.

FIGS. 25A and 25B show a non-circular cover portion being used to seal the ostium of the LAA.

FIGS. 26A-26C, 26D1, 26D2, 26E, 26F1, and 26F2 show an example of a device with proximal and distal anchoring system deployment through a sheath.

FIGS. 27A and 27B show an example of a device once released from a sheath and expanded in the left atrial appendage.

FIG. 28 is an example of signals read from sensors with time delay analysis.

FIG. 29 shows an example of a device with strain gauge sensors embedded in the cover portion.

FIG. 30 is a simplified functional block diagram of an example of using time delay analysis from sensors to measure left atrial pressure.

### DETAILED DESCRIPTION

Before the examples are described, it is to be understood that the invention is not limited to the particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein may be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds, and reference to “the polymer” includes reference to one or more polymers and equivalents thereof known to those skilled in the art, and so forth.

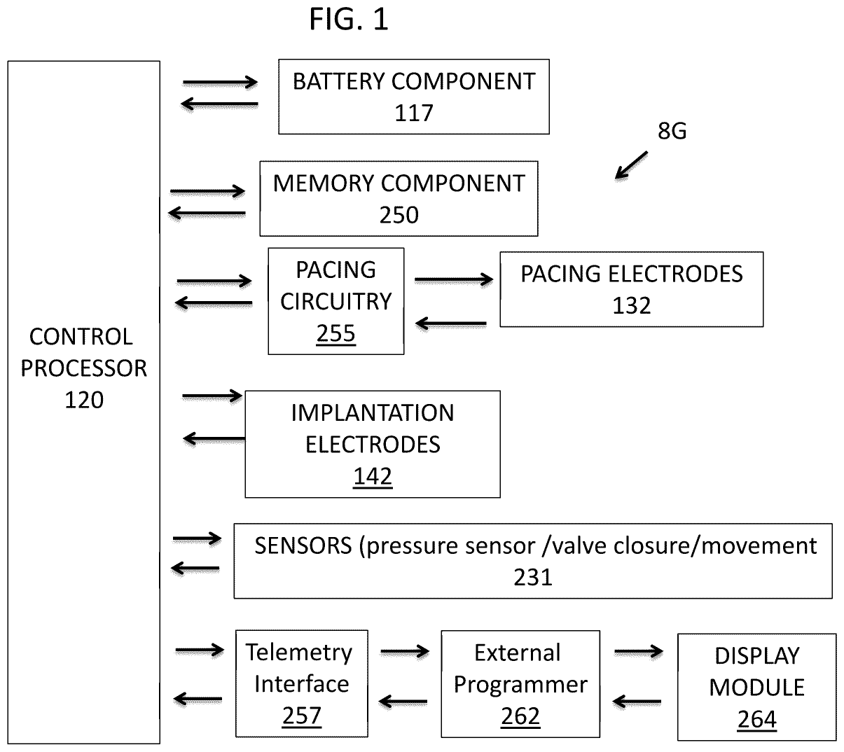

Turning to the drawings, FIG. 1 shows a simplified functional block diagram of an example of a pacemaker device 8G. The device 8G may include a control processor 120, battery component 117, memory component 250, pacing electrodes 132, implantation electrodes 142, various sensors 231, and a telemetry interface 257. The control processor 120 receives input information from various components to determine the function of the different components to treat the patient. The pacing electrodes 132 are used to sense and pace the heart. The pacing electrodes 132 are coupled to pacing circuitry 255, which is coupled to the control processor 120.

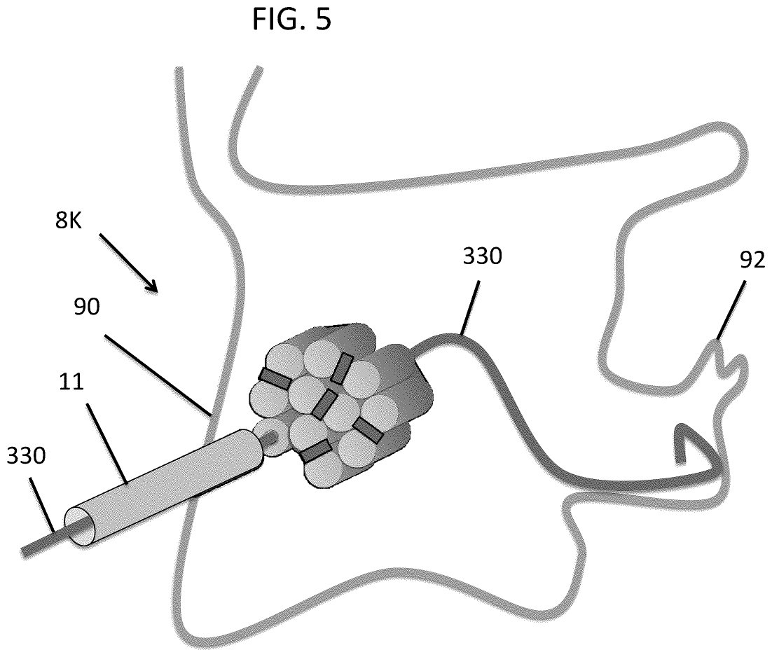

Optionally, the device 8G may include one or more implantation electrodes 142, which may be visualized by a mapping system to help deploy the device 8G within the LAA 92 (not shown, see, e.g., FIG. 5), similar to other examples herein. In addition, optionally, the implantation electrodes 142 may be used to deliver electrical or electroporation energy to electrically isolate the LAA 92. In other examples, the implantation electrodes 142 may deliver energy to help attach the electrodes 142 to LAA 92 tissue to prevent device 8G embolization.

The processor 120 may also be connected to one or more sensors 231. In one example, the sensor(s) 231 may include a three-axis accelerometer. Signals from the three-axis accelerometer may be used by the processor 120 to detect patient activity in the presence of cardiac motion. Alternative sensors 231 may include vibration or movement sensing abilities, which may sense sound or vibrations, e.g., to correlate with valve closure. By determining valve closure, the device 8G may determine what the ventricle of the heart is doing. In another example, one or more temperature and/or movement/accelerometer sensors may be provided that may be coupled to the processor 120 to determine if the patient is exerting or moving in order to determine the pacing rate of the device 8G. In some examples, cardiac motion is used to generate electrical power that can be used to charge or power the device 8G (or any of the other devices herein). For example, the device may include an electromagnetic generator configured to convert the motion of the device due to the beating of the heart into electrical energy, which may be stored in a battery and/or used directly to power electrical components of the device.

In another example, the device 8G may include one or more sensors 231 that correlate with the blood pressure within the left atrium 90. These sensor(s) 231 may help identify a heart failure admission similar to the CardioMEMs™ device. These sensor(s) 231 may also be used to optimize medical therapy. In another example, various measurements between electrodes may be used to guide device 8G therapy. Both near-field and far-field electrical activity may be used to determine atrial and ventricular activity as well as diagnose conduction abnormalities.

In some examples, the device 8G (or any of the other devices herein) may include one or more electrodes configured to measure impedance. The impedance may be used to estimate left atrial pressure within the heart. For example, the device may include a cover or cap (not shown, e.g., similar to the covers described elsewhere herein for covering the entrance to the left atrial appendage) and may include electrodes configured to measure impedance, resistance, acoustics, optics, and/or deflection. For example, as the cap changes shape due to pressure or pressure waveforms, the impedance between the electrodes may change and a processor coupled to the sensors may process signals reflecting the changes in impedance and estimate the pressure. The left atrium pressure is not static and varies with the cardiac cycle, patient movement, and respiration. As the left atrial pressure increases, there are corresponding impedance changes. In addition, the changes in impedance throughout the cardiac cycle may also change. Therefore, there will be absolute impedance changes and a relative change in impedance measurements during the cardiac cycle. These changes may be used to estimate left atrial pressure.

Optionally, the device may have the ability to calibrate the sensors that correlate with pressure. For example, the patient may undergo certain maneuvers or position changes or go into a pressurized chamber. These maneuvers or places and the resultant sensor changes may be used to calibrate the pressure estimations. This pressure may be monitored by the processor of the device and/or transmitted externally, e.g., using a wireless transmitter or other communications interface (not shown) to guide medications, especially diuretics. In another example, the cap or another component of the device 8K configured to change shape as the left atrium pressure changes, may include a strain gauge coupled to a processor of the device. In this example, the processor may analyze signals from the gauge, e.g., related to piezoresistive, capacitive, or impedance-based effects, to measure changes in the shape, and the changes in shape may be used to measure the left atrial pressure.

The impedance measurements along the cap may also be used to determine and monitor the development of endothelialization or device-related thrombus or DRT. As the cap is gradually endothelialized, the impedance measurements should follow a predictable change over time. If the impedance trends over time may be used to alarm the physician, patient, or other care team member to evaluate the pacemaker device within the left atrial appendage.

In addition or alternatively, the device 8K may be configured to measure or estimate the amount of blood flow within the left atrium. In another example, the device may have a sensor that may estimate the risk of developing thrombus formation within the left atrium. This information may help inform the physician or patient about the risks and/or benefits for the patient to be on blood thinners. Optionally, one or more additional sensors, pressure measurements, atrial rhythm, heart rate, patient activity, temperature, etc., may be included in the device to provide other clinical information (prior history, patient age, etc.) to these parameters to create a risk score for whom should be on anticoagulation or whom anticoagulation can be safely stopped.

The pacing circuitry 55 connects to pacing electrodes 132 to the control processor 120. These connections allow for multiple capacities to sense electrical activity (such as myocardial depolarizations), deliver pacing stimulations, and/or deliver defibrillation or cardioversion shocks. The control processor 120 may be connected to a telemetry interface 257. The telemetry interface 257 may wirelessly send and/or receive data from an external programmer 262, which may be coupled to a display module 264 in order to facilitate communication between the control processor 120 and other aspects of the system external to the patient.

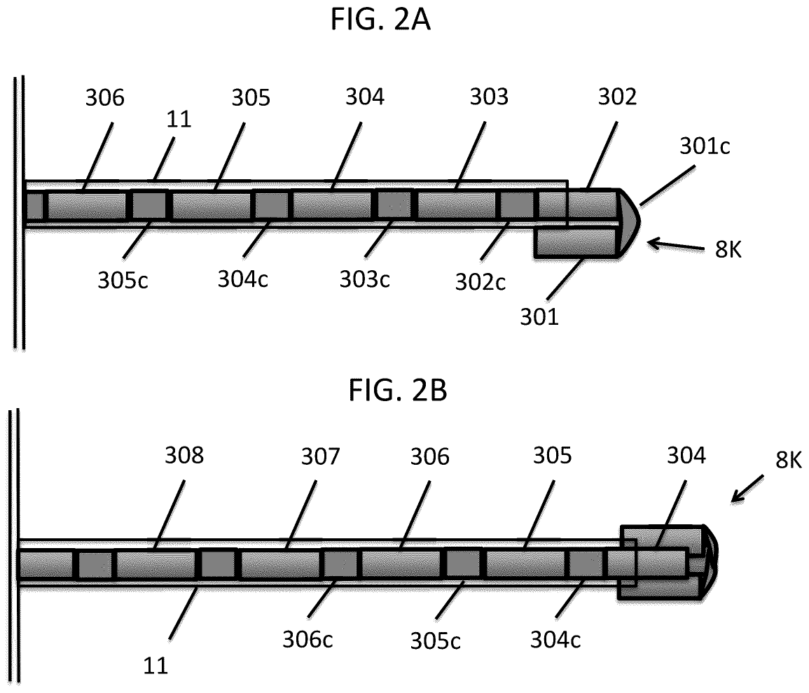

Turning to FIGS. 2A-2B, an exemplary method is shown for deploying a device 8K when the device 8K is advanced out of a delivery sheath 11. Initially, a distal subunit 301 is deployed from the delivery sheath 11, whereupon a connector 301c to an adjacent subunit 302 provides a hinge point, e.g., biased to cause the distal subunit 301 to have an orientation change to the adjacent subunit 302. For example, the connector 301c may be biased to a “U” or other shape, e.g., to cause the distal subunit 301 to rotate or otherwise translate one hundred eighty degrees relative to the second subunit 302. Moving onto FIG. 2B, as the device 8K is further advanced out of the delivery sheath 11, the subunits 302-311 are deployed sequentially, and automatically fold relative to the adjacent subunits into a designed orientation. In some examples, the connectors 302c-311c of the device 8K are configured to automatically orient the subunits 302-311 as the device 8K emerges from the delivery sheath 11.

In other examples, an external force may be used to force the change in orientation. For example, the connectors 301c-311c may have a hinge-type joint where the degrees of freedom are designed to fold the device 8K into the pre-designed orientation.

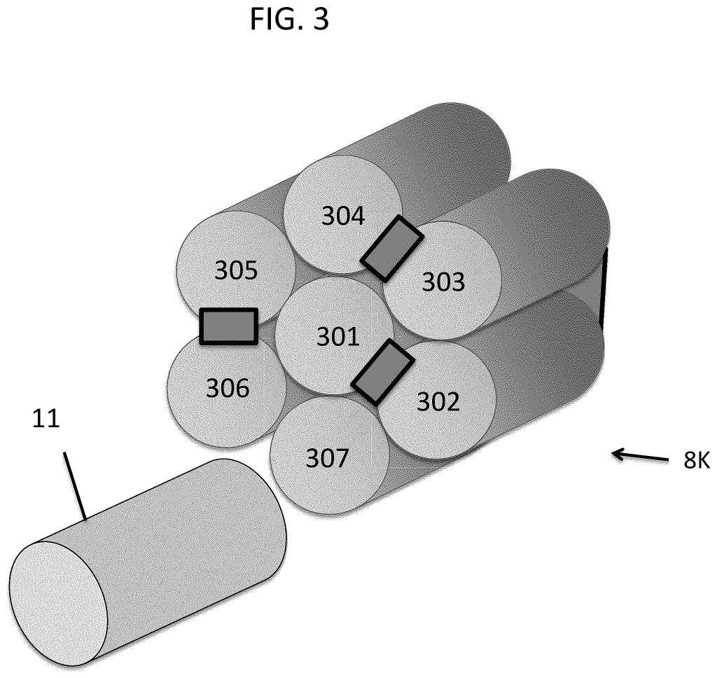

FIG. 3 demonstrates an exemplary expanded configuration where the subunits 301-311 orient themselves into a structure depicted, e.g., where the subunits 301-311 are aligned axially adjacent to one another with the connectors 301c-311c alternating between opposite ends of the expanded structure. In this example, the individual subunits 301-311 are substantially rigid; while the connectors 301c-311c enable the device 8K to fold into the depicted structure. In other examples, there may be more or fewer subunits and connectors, and they may configure into different morphologies than shown here.

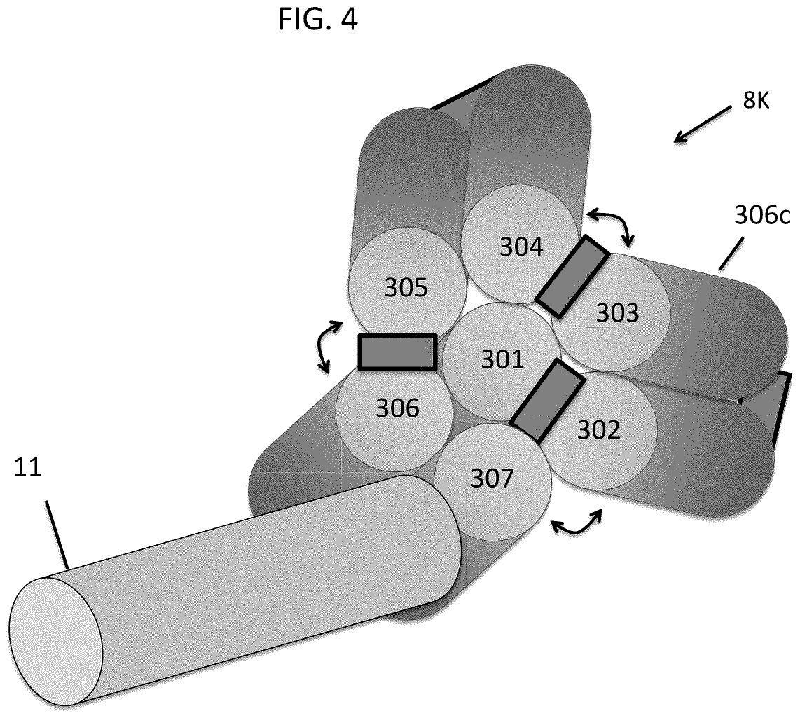

FIG. 4 demonstrates another exemplary expanded configuration of the device 8K where the subunits 301-311 extend radially outwardly from a central region, to provide a conformational change once positioned inside the LAA (not figured). In this example, the connectors 303c, 305c, 307c, 309c, and 311c are designed to expand along their hinge joint, while other connectors 301c, 302c, 304c, 306c, 308c, and 310c are designed to maintain a closed position.

Optionally, one or more tines, anchors, and/or leads (not shown) may be provided on the connectors 304c, 306c, 308c, and 310c that make contact against the LAA (not shown), e.g., to prevent embolization or to confirm contact through electrical signals, similar to other examples herein. In other examples, the connectors 304c, 306c, 308c, and 310c may include electrodes (not shown) that may be used for visualization, cardiac pacing, tissue heating (for tissue ‘sticking’), ablating, and/or electroporation, as previously described elsewhere herein.

Optionally, any of the devices herein may include one or more sensors configured to contact the LAA when the device(s) are implanted. In some examples, a processor coupled to the sensors may be configured to determine the amount of force pushing against the heart tissue, which may be communicated to a location outside the patient's body, e.g., using a wireless transmitter or other communications interface (not shown). For example, an external system may receive information from the sensors and display the amount of force the device is putting against the heart tissue to make sure the force is adequate to stabilize the device. Therefore, in some examples, during the introduction and deployment of the device, the physician may slowly enlarge the device until the amount of measured force is adequate to anchor the device within the left atrial appendage.

Optionally, any of the devices herein may include a mechanism to anchor the device into the left atrium. For example, the device may include using one or more pins, pinches, screws, rivets, hook and eye fasteners, snaps, clamps, and/or adhesives or other mechanical fixtures to anchor the device into the heart. In another example, electrical energy may be delivered to one or more electrodes carried on the device near or in contact with the heart. For example, electrocautery may be delivered to one or more electrodes in contact with heart tissue to heart the cardiac tissue. Alternatively, the energy delivered may include one or more of laser, radiofrequency energy, heat, ultrasonic energy, and the like to cause adhesion. Furthermore, if the anchoring mechanism has caused some bleeding (for example the anchoring tines have punctured cardiac tissue), the energy delivery may be used to cause hemostasis and stop the bleeding. The amount of energy delivered may be delivered using standard ablation techniques.

For example, the power and voltage may be delivered using a feedback loop while measuring changes in impedance, temperatures, current, power, voltage, phase, and/or time. The heat may be used to cause protein denaturing (e.g. collagen and elastin proteins) to attach to the electrode. Therefore, electrical energy can be delivered in order to help anchor the device within the left atrial appendage. Additionally, heat can be used to help attach the cover portion to the left atrium. Electrical energy may also be delivered to change the impedance between an electrode and atrial tissue. The electrical energy may be used to create heat, which will affect the interaction between the electrode or anchor and the atrial tissue. The changes in impedance may decrease the amount of energy required to capture the tissue to prolong battery life or reduce the amount of charging is required.

In another example, a component or portion of the device configured to contact heart tissue when the device is implanted may be covered with material that facilitates adhesion. Optionally, the adhesive mechanism may be activated by blood, electrical energy, time, and/or heat. Optionally, the cover or cap may also be coated with a material that facilitates endothelialization.

In another example, any of the devices herein may include at least three anchoring portions (not shown) configured to be expanded radially to anchor the device 8K within the left atrial appendage 92. The anchoring portion(s) may include one or more tines and/or electrodes that facilitate anchoring. The tines (not shown) may be protected or not exposed as the device 8K re-arranges in the left atrium or left ventricle. Later, as a radial force is used to expand the anchoring portions, these tines are then exposed to atrial tissue to facilitate anchoring.

In other examples, energy delivered to one or more electrodes on the device may be used to expose the tines for anchoring. This may include using electrical energy to move and change the angle of the tines. For example, heat or electrical energy may change a tine made out of Nitinol or other shape memory material to change in such a way as to facilitate anchoring and contact with atrial tissue. In other examples, it is the turning off of electrical energy that changes the tine for anchoring. The electrode or tine may also be able to measure impedance. In other examples, the tine or electrode may include a sensor that corresponds with pressure. Therefore, the pressure sensor or impedance or other sensor may be used to estimate the amount of force against the tissue. In addition, the device may include one or more sensors configured to generate signals related to pressure, which may be used to guide the expansion of an anchoring mechanism to anchor at least a portion of the device 8K within the left atrial appendage 92.

Alternatively, the example shown in FIG. 4 may represent the configuration of the device 8K wherein initially deployed within the left atrium, e.g., when fully deployed from the sheath 11. Features such as tines, hooks, loops, or the like (not shown) on the distal portion of the connectors 304c, 306c, 308c, 310c may be manipulated to narrow the device profile to the configuration presented in FIG. 3 for advancement into the LAA 92.

For example, with reference to FIG. 5, the device 8K may be deployed within the left atrium 90 in the configuration shown in FIG. 4 and then folded or otherwise constrained into the configuration shown in FIG. 3, whereupon the folded device 8K may be advanced into the LAA 92, e.g., over guide 330. In this example, the delivery sheath 11 has been advanced into the left atrium 90, e.g., through the interatrial septum 94. Once inside the left atrium 90, the elongate device 8K may be advanced to create the desired shape within the left atrium 90, e.g., the deployed configuration shown in FIG. 4. Similar to a ship-in-a-bottle, the device 8K may be manipulated to take on a conformational change within the left atrium 90 to take on a desired shape or structure, e.g., that shown in FIGS. 3. The conformed device 8K may then be advanced into the LAA 92. Once inside the LAA 92, the device 8K may take on a second conformational change to deploy the device 8K into the LAA 92, e.g., by releasing the device 8K to allow the subunits to resiliently return towards the configuration shown in FIG. 4. The second conformational change locks the device 8G in place within the LAA 92.

In the example depicted in FIG. 5, an elongate guide member 330 is provided that is configured to pass through a delivery lumen of the delivery sheath 11, the proximal connector 311c (not shown), and the proximal subunit 311. In one example, the elongate guide member 330 may be a wire or may be a steerable catheter. In other examples, the elongate guide member 330 may be similar to a pigtail catheter with an inner lumen that allows an inner wire to advance through the elongate guide member 330. For example, a distal portion of the elongate guide member 330 may be exposed from the sheath 11 and advanced or otherwise directed into the LAA 92, and then the device 8K may be advanced over the elongate guide member 330 in order to position the device 8K optimally within the LAA 92. In some examples, the elongate guide member 330 passes through an inner lumen of the connector 311c and subunit 311 or connector 301c.

In some examples, at least the distal portion of the elongate guide member 330 may have a substantially square or other non-circular cross-section. By being square, the elongate guide 330 may be spun to deliver force to the device 8K. For example, if the elongate guide member 330 and an inner lumen of the subunit 311 have a similar cross-section, rotating the elongate guide member 330 about its longitudinal axis may be used to spin the subunit 311. This spinning motion may be designed to expand the device 8K into a desired expanded configuration, for example, the configuration shown in FIG. 4.

In other examples, the elongate guide member 330 may include a balloon or other expanded member on the distal portion that may be inflated or otherwise expanded to expand or otherwise deploy the device 8K once in place in the LAA 92. The balloon portion may be asymmetric in order to expand the distal or proximal connectors. In other examples, the elongate guide member 330 may also include docking features (not pictured) to interface with the distal portion of the device 8K, e.g., providing an additional landmark for visibility during device placement.

Alternatively, other mechanisms may be provided to orient the device 8K, e.g., including one or more of springs, ratchets, Nitinol or other elastic material, temperature-activated materials, and/or through electricity. In some examples, one of the connectors used to orient the device 8K may also serve as an atraumatic lead in the tip of the delivery sheath 11.

In other examples, a string or wire (not shown) may run through the device 8K. The string may be pulled or otherwise actuated from outside the patient, e.g., using an actuator on the proximal end (not shown) of the sheath 11, to force the subunits to collapse as or after the device 8K is advanced out of the delivery sheath 11. Once the device 8G is positioned within the LAA 92, the string may be relaxed. The relaxation may then cause certain connectors (or all the connectors) to elongate in prescribed directions and/or force to lock the device 8K within the LAA 92. For example, once the string is relaxed, the device 8K may expand automatically through a variety of methods, including but not limited to a spring mechanism, Nitinol, temperature-activated materials, and/or electrical energy. Optionally, the string may be tightened to collapse the device 8K in a desired manner. Repositioning within the LAA 92 while in the narrowed configuration of FIG. 3 may be repeated as needed, by relaxing the string to expand the device 8G, verifying the position, and tightening the string to reposition as necessary. After the string is relaxed and placement within the LAA 92 is confirmed, the string may then be cut or otherwise separated and withdrawn from the device 8K to leave the device 8K in place within the

LAA 92.

In another example, a balloon may be provided on a distal tip (not shown) of the elongate guide member 330, which may be filled with saline and/or other inflation media to expand the subunits 301-311 of the device 8K in a desired manner, e.g., opening the proximal connectors to assume the shape in FIG. 4, creating contact with the distal portion of the device 8K and the LAA 92. If the device 8K needs to be advanced into the LAA 92, the balloon may be inflated within the proximal connectors, thereby expanding the proximal profile of the device 8K and narrowing the position of the distal connectors. The balloon may then be collapsed, the device 8K advanced further into the LAA 92, and the balloon inflated within the distal connectors. In another example, features on the distal connectors remain captured by the delivery system using a string method similar to that described above, and are leveraged to collapse the device after balloon expansion.

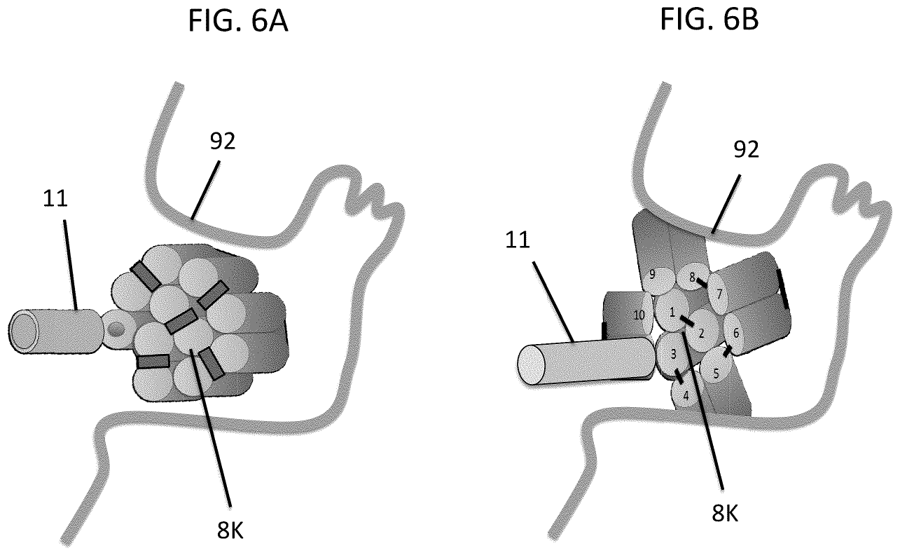

Turning to FIGS. 6A and 6B, an exemplary method is for implanting the device 8K, i.e., deploying, introducing and anchoring the device 8K within the LAA 92. Initially, the device 8K is advanced into the LAA 92 in a constrained condition, e.g., similar to the configuration shown in FIG. 3 Moving onto FIG. 6B, the device 8K may then be enlarged or otherwise deployed to connect within the walls of the LAA 92, e.g., similar to the configuration shown in FIG. 4. The device 8K may then undergo tug testing from outside the patient's body to make sure the device 8K is adequately fixed within the LAA 92, visually assessed using fluoroscopy, echocardiography, or other visual mapping methods to confirm device 8G shape and depth of position, and/or contact with the LAA may be confirmed by electrical signal. If needed, the device may be recaptured, repositioned, and then re-deployed, e.g., using any of the methods described elsewhere herein. In another example, if fixation is insufficient, the proximal hinges may be further elongated or otherwise manipulated in order to widen the angle between the rigid subunits, providing greater apposition to the LAA 92.

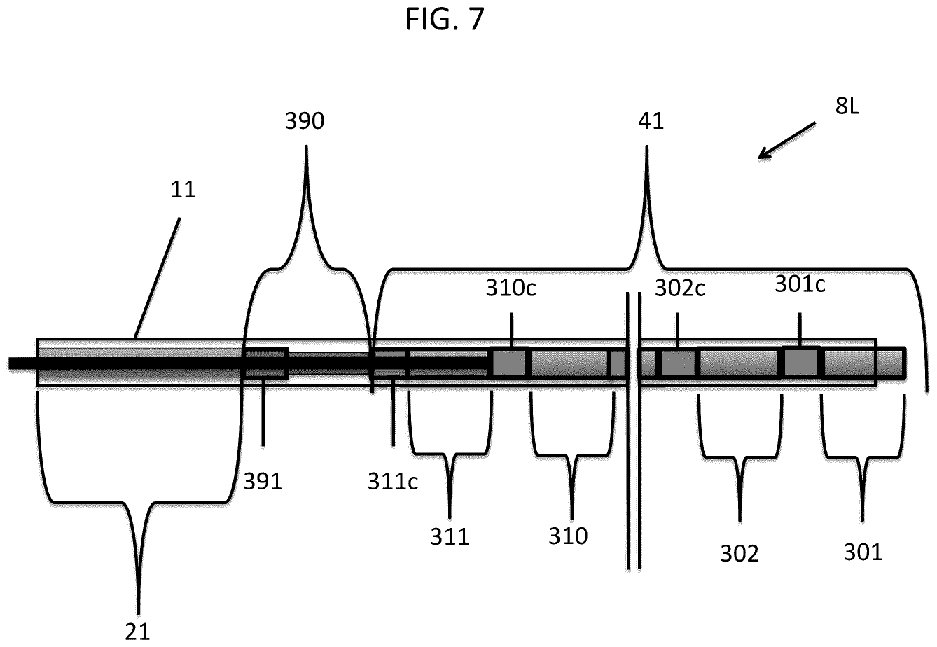

FIG. 7 shows another example of a pacing device 8L designed for implantation with the LAA 92. In this example, the device 8L includes three components, segments, or portions that are introduced, deployed, and remain within the vicinity of the LAA 92: an occluding or cover portion 21, a coupling portion 390, and an anchoring portion 41. The components of the device 8L may be introduced and/or deployed using a deployment sheath or other tubular and/or elongate member 11. The components may be separate devices that may be deployed sequentially or they may be components of a single integral device including different regions. In some examples, the delivery sheath 11 may be biased to a predetermined shape, e.g., including a bend or curve to facilitate the components of the device 8L being deployed from the deployment sheath 11 in a controlled method. The delivery sheath 11 may be adjustable or steered from outside the patient to facilitate delivery of the device 8L into the LAA 92.

With continued reference to FIG. 7, the anchoring portion 41 may be made up of repeating subunits 301-311, e.g., connected sequentially by connectors 301c-311c, similar to other examples herein. The connectors 301c-311c connecting the subunits 301-311 may be flexible to allow the subunits 301-311 to move in a desired manner, e.g., during advancement through the delivery sheath 11, yet bias the device 8L to adopt a desired shape when deployed. For example, the connectors 301c-311c may provide hinge points that permit the subunits 301-311 to fold on themselves to provide a desired expanded configuration. Optionally, the connectors 301c-311c may include one or more tines (not shown), electrodes (not shown), or other components to facilitate deployment, e.g., similar to other examples described elsewhere herein.

The coupling portion 390 is designed approximate the occluding portion 21 to the anchoring portion 41. The coupling portion 390 contains a coupling actuator 391 that enables the distance between the occluding portion 21 and the anchoring portion 41 to be adjustable. Therefore, once the anchoring portion 41 has been anchored within the LAA 92, the coupling portion 390 may be adjusted to make sure the occluding portion 21 completely covers the LAA 92. The mechanism of controlling this distance may be a screw mechanism or a motor. In some examples, a string is used to pull the occluding portion 21 to the anchoring portion 41. In other examples, a spring mechanism automatically pulls the occluding portion to the anchoring portion. The coupling mechanism may enable rotation of the occluding portion with respect to the anchoring portion.

The occluding or cover portion 21 may also be lined by or otherwise include a plurality of electrodes. The occluding portion 21 may be a self-expanding disc to close off the LAA 92 from the rest of the left atrium (“LA”) of a subject's heart. In another example, the occluding portion 21 is biased to a predetermined helical and/or conical shape, e.g., that spirals on itself like a pyramid, that may be shaped to completely close off the LAA when deployed. The cover portion 21 (or any of the other covers described herein) may be substantially circular or, alternatively, the cover portion 21 may have an oval or other non-circular, e.g., symmetrical shape. Optionally, the occluding portion 21 may be made of or covered in a certain material that facilitates endothelialization and/or minimizes platelet aggregation. The occluding portion 21 may be elliptical in shape and the coupling attachment site may be in the center or off-center in order to make sure the occluding portion 21 completely closes off the LAA 92. In another example, the occluding portion 22 has electrodes that may be used to measure impedance across the occluding section 21, e.g., to monitor for complete coverage of the LAA 92.

In some examples, the device may include one or more sensors to measure pressure attached to or embedded in the proximal side of the cover portion 21 facing the LA 90, on the distal side of the cover portion 21 facing the LAA 92, or between the cover portion 21 and anchoring portion 41. If the cover portion is fully endothelialized, this may prevent LA-facing pressure sensors from measuring the pressure accurately, or a pressure sensor in the LA 90 may increase the risk of stroke. Where a pressure sensor is facing the LA 90, the sensor material may be protected to prevent endothelialization over the diaphragm of the sensor. In some examples, strain gauges to measure changes in the deflection of the endothelialized tissue may be used on the cover portion inside the LAA or on the coupling portion. The strain gauge may be made of piezoresistive, capacitive, or impedance-based materials that transmit electrical signals to an external receiver. As the cover portion and endothelialized tissue flex in response to changes in

LA pressure, the resistance, capacitance, or impedance of the sensor will be altered. In some examples, the device may include an ultrasonic transducer that sends signals from the LAA to the LA through the endothelialized tissue to a receiver, measuring the LA pressure acoustically by measuring parameters such as amplitude, phase, or time of flight. In other examples, an optical sensor may be used to measure pressure changes by detecting alterations in the optical properties (e.g., refractive index, wavelength, or intensity) of the sensor.

Therefore, in some arrangements, the system includes a sensor that comprises an impedance sensor coupled between the cover portion and the anchoring portion. The left atrial pressure than is transmitted to the cover portion. By having the sensor coupled between the cover portion and the anchoring portion, the measured pressure can be used to in part to estimate left atrial pressure. The sensor can be coupled to the processor and used to detect changes in pressure by measuring changes in impedance, capacitance, and/or resistance.

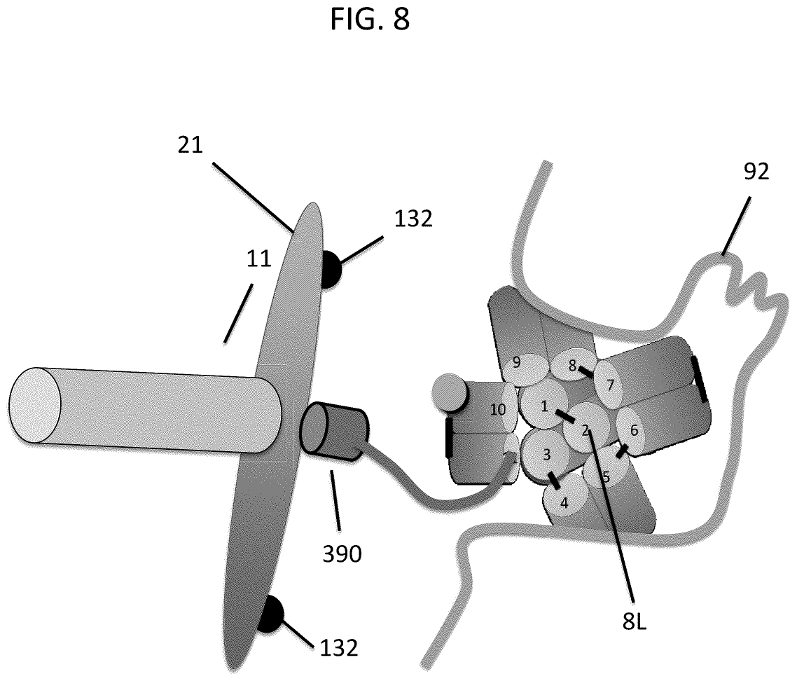

Moving on to FIG. 8, once the anchoring portion 41 is satisfactorily deployed within the LAA 92, a cover or occluding portion 21 may be deployed to cover the ostium of the LAA 92. The cover 21 may be made out of a variety of materials. The cover 21 is designed to cap the ostium of the LAA 92 and prevent thrombus or clot that may form within the LAA 92 from leaving the LAA 92. In some examples, the cover 21 includes one or more pacing electrodes 132, e.g., located on the LAA 92 side of the cover 21. These pacing electrodes 132 may be configured to contact the wall of left atrium 90 outside of the LAA 92. Therefore, if the LAA 92 is electrically isolated, the pacing electrodes 132 are still able to sense and capture atrial tissue.

The pacing electrodes 132 may be single or bipolar electrodes. The pacing electrodes 132 are therefore able to sense atrial depolarizations and deliver pacing stimulations to pace the atrial tissue. In some examples, atrial anti-tachycardia pacing (ATP) may be delivered from one electrode 132 and sensed by other electrodes. Therefore, pacing stimulations may be delivered at one location; and distant electrodes are able to determine if the pacing stimulations are capturing heart tissue. The ATP algorithm may then change pacing strategies based on whether the pacing stimulations are capturing the atria.

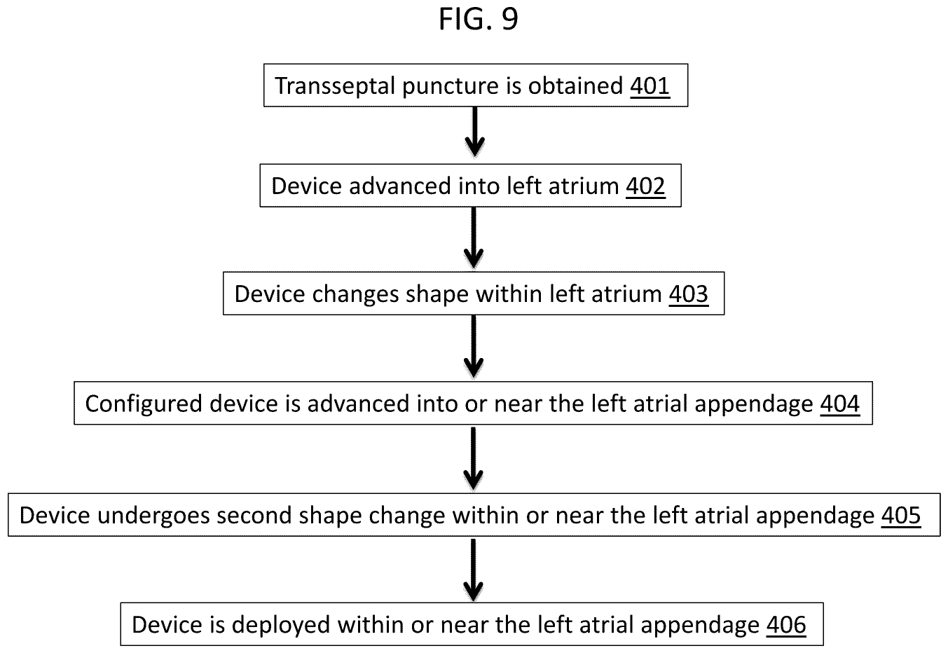

Turning to FIG. 9, a flow diagram is shown illustrating an exemplary method for implanting a device, such as the device 8K (or any other devices herein). In step 401, access of the left atrium is obtained. A delivery sheath is typically placed across from the right atrium into the left atrium. In step 402, the device is advanced into the left atrium. Moving to step 403, once part of the device is located within the left atrium, and the device changes shape (e.g., automatically upon deployment or upon being actuated). The shape change may be described as a conformation change or a configuration change. Moving to step 404, the conformed or configured device may then be advanced into or near the left atrial appendage. In step 405, the device may then undergo a second shape change within or near the left atrial appendage (e.g., constrained or otherwise manipulated into a smaller profile). This second shape change may also be described as a second conformational or second configuration change. In some examples, this second shape change is utilized to keep the device within or near the left atrial appendage. In step 406, the device may then be deployed within or near the left atrial appendage. The delivery tools may then be removed from the left atrium.

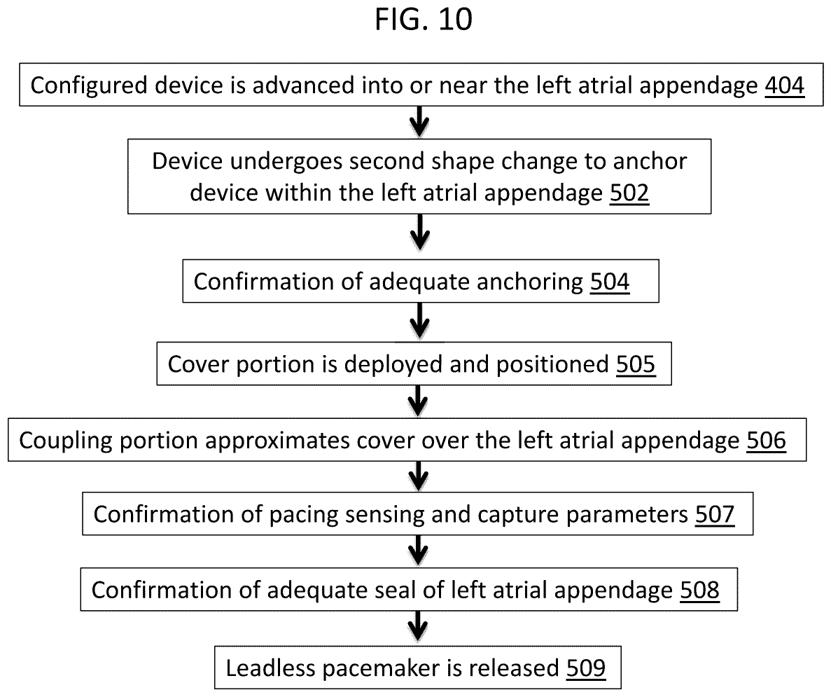

FIG. 10 is another flow diagram describing another exemplary method for implanting a leadless pacer into the left atrial appendage. Following Step 404 from FIG. 9; the procedure may advance to Step 502 where the device undergoes a second shape change within or near the left atrial appendage to anchor the device within the left atrial appendage. The device may be anchored by radial force, or tines, or a combination. In other examples, additional anchoring mechanisms may be utilized. Moving to step 504, there is then confirmation that the of adequate anchoring. In one example, the coupling portion may perform a tug test to verify the anchor is firmly anchored within the left atrial appendage. In other examples, the degree of compression may be utilized. In another example, the electro-anatomical map may be analyzed to verify adequate anchoring. In one example, at least one electrical metric or metric from the electro-anatomical map is utilized to verify adequate anchoring.

Moving on to step 505, the cover portion is deployed and positioned within the left atrium. In some examples, the cover may be rotated such that the pacing electrode(s) land against the optimal position in the left atrium. For example, the cover may be carried by the device such that the cover may be rotatable relative to other components of the device. Next in step 506, the coupling portion is activated to approximate the cover over the left atrial appendage. In some examples, a screw mechanism adjusts the distance between the anchor and the covering portion. In other examples, a string or electrical motor may be utilized. In yet another example, a spring mechanism is utilized.

Moving on to step 507, confirmation of pacing sensing and capture parameters is performed. Next in step 508, confirmation of adequate seal of the left atrial appendage is performed. In one example, the electro-anatomical map can be analyzed to verify adequate seal. In another example, at least one electrical metric or a metric from the electro-anatomical map is utilized to verify adequate covering or seal of the left atrial appendage. Moving on to step 509, once the device has achieved all implantation criteria, the device can be released.

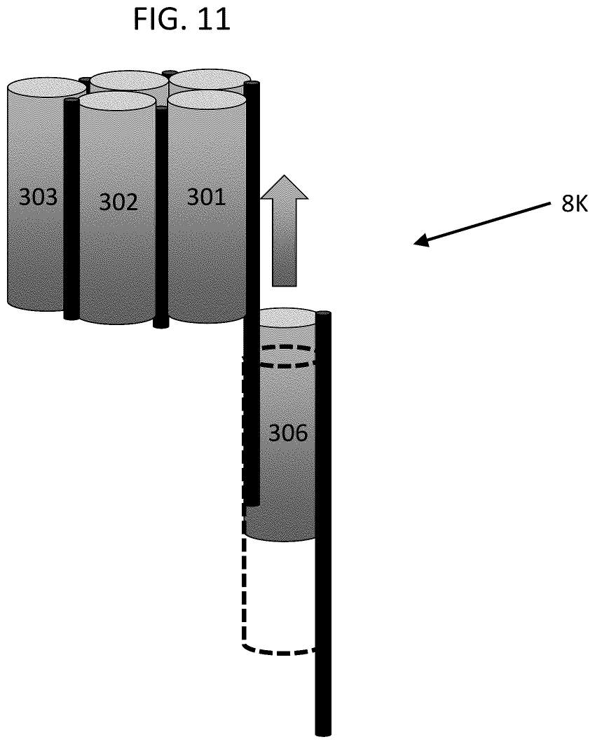

FIG. 11 demonstrates an exemplary expanded configuration of a leadless pacemaker device 8K that includes a plurality of subunits 301-311 (e.g., connected together similar to other devices herein) that orient themselves into a structure as depicted, e.g., where the subunits 301-311 are aligned axially. In this example, the subunits translate adjacent one another such that one subunit can slide parallel to its neighboring subunit. This conformational change can occur in the left atrium. In other examples, the conformational change may occur directly within the LAA 92. The subunits may automatically arrange themselves in the desired configuration or may need external forces to arrange in the desired configuration.

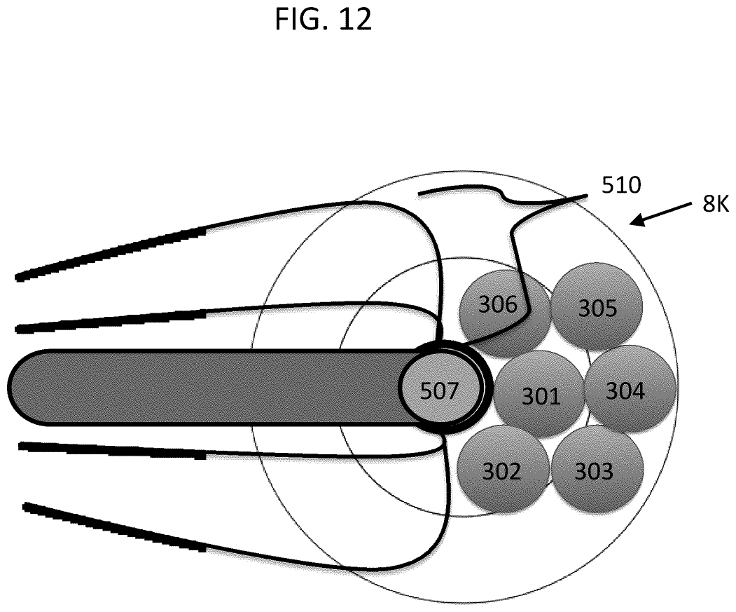

FIG. 12 is a cross sectional view of the leadless pacemaker device 8K being deployed inside the left atrial appendage. In this figure, there is a deployment subunit 507 that anchors the device 8K within the LAA 92. The deployment subunits can have expandable tines 510 that enlarge the device 8K and anchor the device 8K within the LAA 92. The expandable tines 510 may automatically expand when allowed to do so, e.g., by unsheathing or a spring-like mechanism. In other examples, the expanding tines 507 are enlarged by the proceduralist. This could be similar to an umbrella device-where force proximally expands the expandable tines 510. Furthermore, the amount of force placed on the expandable tines 510 may be titratable. For example, the proceduralist may be able to gradually enlarge the expanding tines 510 to a desired amount. Determining when to stop expanding the expandable tines 510 could be via one of more sensors, e.g., a pressure sensor located on the device 8k (not shown). In addition, by knowing the size of the device in relation to the number of turns delivered by the proceduralist may also determine when enough radial force has been delivered. The expandable tines 510 may be made of Nitinol or other elastic material, which can be deployed and then automatically expand. The expandable tines 510 can also be ‘re-captured’ into a contracted state should repositioning be desired.

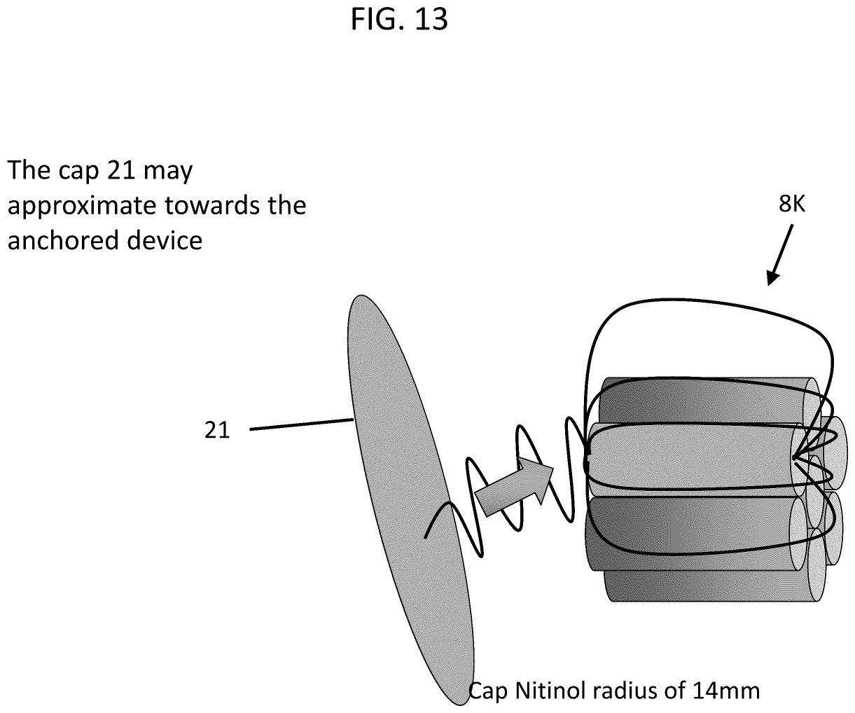

FIG. 13 is another schematic illustration of an example of the leadless pacemaker device 8Kk with a cover portion 21 over the LAA 92. In this example, the deployment subunit 507 has been expanded with the expandable tines 510 adjacent to the LAA 92. The subunits 302 and 306 can be seen well positioned within the LAA 92. The cover portion 21 can then be deployed to cover the surface of the LAA 92 ostium to close off potential thrombus or clot from within the LAA 92 to embolize out of the heart. The cover portion 21 may be attached to the deployment subunit 507 by a cover connector 570 that automatically approximates the cover portion 21 over the LAA 92 ostium. In some examples, the cover portion 21 is approximated to the device 8K by the proceduralist; in other examples, the cover connector 570 utilizes an automatic force, such as a spring, in order to land the cover portion 21 over the LAA ostium 92. The proceduralist may have the ability to tighten or loosen the connection or reposition the cover portion 21 to the desired location. The cover portion 21 may be a single cohesive subunit or may comprise numerous individual components, all of which prevent blood clots from leaving the LAA 92.

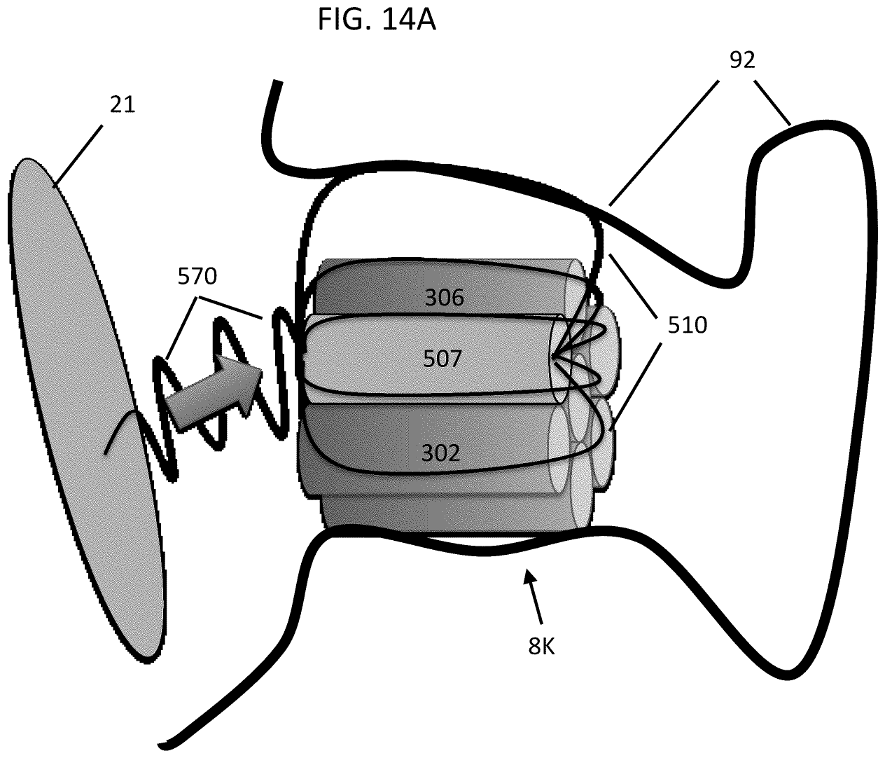

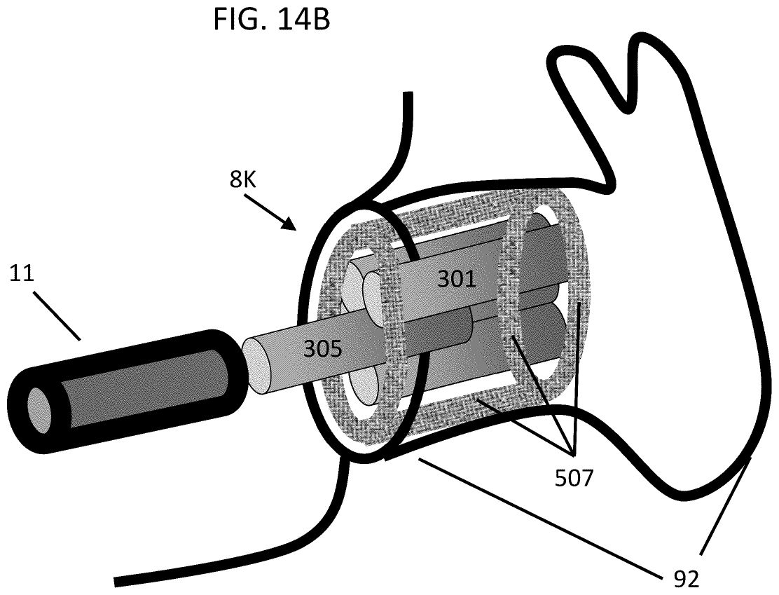

FIGS. 14A and 14B are cross-sectional views of a left atrial appendage 92 showing the leadless pacemaker device 8K of FIG. 13 being deployed inside the left atrial appendage 92 after the anchoring subunit 507 has been deployed. In this example, the anchoring subunit 507 was anchored first into the LAA 92. After the anchoring subunit 507 is deployed, other subunits (subunits 301 and 305 are labeled) can be deployed inside of the anchoring subunit 507. In this example, the delivery sheath 11 can be used to fill the inside of the anchoring subunit 507. Here, the subunit 305 can translate next to neighboring subunits into the LAA 92 and inside the anchoring subunit 507. In some examples, a cover portion 21 (not shown) can be placed adjacent to the other subunits. In other examples, the outside-facing subunits may have a certain covering that facilitates tissue growth and/or prevents thrombus formation on the face of the device 8K.

In another circumstance, the device 8K may communicate or be charged by another implanted device located more superficially in the body (not shown). For example, the device 8K may be charged via a device implanted in the left shoulder. The superficial device can send ultrasound or other energy modalities to charge the device 8K. The superficial device can also communicate to other devices external to the body. The superficial device can then be charged via inductance or other modalities to enable the device 8K to have a longer battery life.

In another example, the device 8K may function with a leadless device that paces the ventricles (not shown). The device 8K can send pulses to communicate with the leadless device in the ventricle. In other examples, the functioning of the device 8K may be programmed to facilitate function with other devices. For example, the pulses delivered by the device 8K may be sensed by a leadless ventricular pacemaker. In order to coordinate timing of pacing, the device 8K can deliver a pulse upon sensing an atrial depolarization. This pulse can be sensed by the ventricular pacemaker in order to determine device functioning.

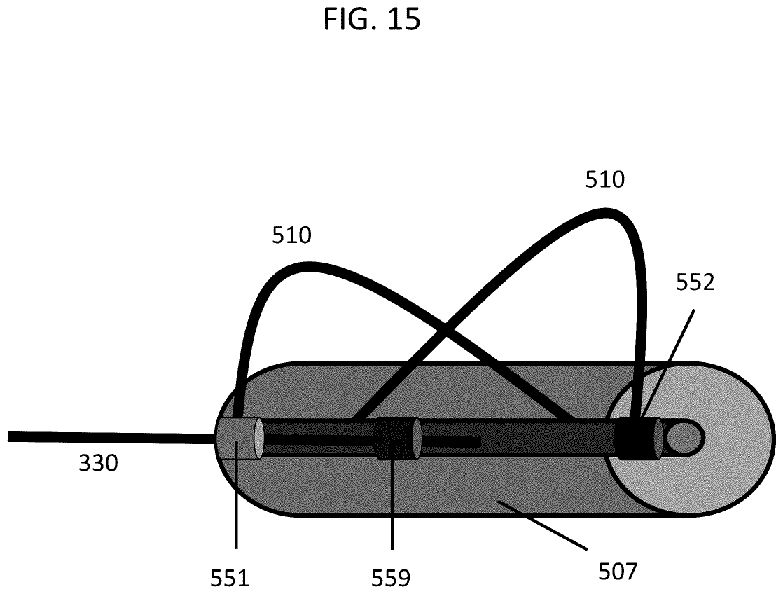

Turning to FIG. 15, the device 8K can be expanded in different locations. For example, the proximal and distal aspects of the anchoring subunit 507 may be enlarged separately. In one example, the elongate guide member 330 can be used to enlarge the tines 510. In this example, the elongate guide member can a component 559 that can engage with other components 551 and 552 that can be used to expand the tines 510. By advancing or pulling back the elongate guide member 330 relative to the device 8K, the component 559 on the elongate guide member may be positioned to engage with the other components 551 or 552. For example, the component 559 can be rotated which engages and also rotates another component 551. When the component 551 rotates, the tines 510 move away from the center of the anchoring subunit 507. The radial force of the tines 510 can be used to anchor the anchoring subunit 507 within the LAA 92. The subunits 551 and 552 can be rotated or moved in different directions to enlarge or contract the tines 510. Pressure recordings can be used to determine when the force is adequate to successfully anchor the anchoring subunit 507 within the LAA 92. The pressure recording may be used with a pressure sensor. In other examples, a mapping system and/or impedance measurements can be used to determine when the force on the tines 507 is adequate to anchor the anchoring subunit 507. Optionally, a ‘tug test’ may be used where the anchoring subunit is pulled away from the LAA 92. The mapping system, impedance measurements, or pressure sensors may be used to determine if the anchoring subunit is adequately anchored within the LAA 92.

In other examples, the tines 510 are held in place by an outer sheath (not shown). When the outer sheath is withdrawn from the anchoring subunit 507, the tines 510 are free to expand and enlarge. The outer sheath (not shown) may be advanced over the tines 510 to collapse the tines 510 should the anchoring subunit 507 need to be repositioned or withdrawn.

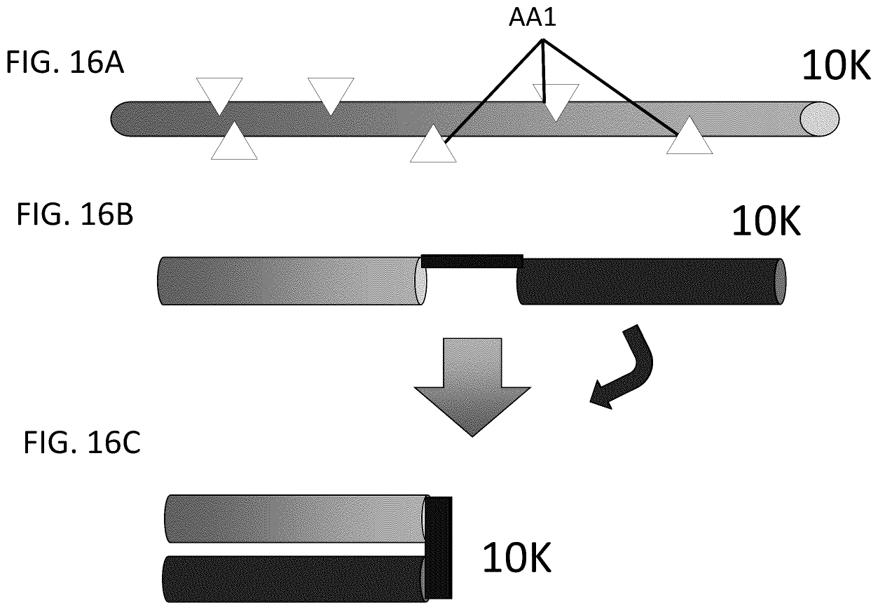

Turning to FIGS. 16A-16C, another example of a device 8K is shown that is configured to be implanted into the LAA, similar to other devices herein. As shown in FIG. 16A, a Nitinol tube 10K is provided with various cuts AA1 made into the tube 10K, e.g., spaced apart from one another along a length of the tube 10K. As shown in FIG. 1B, the Nitinol tube 10K may be folded at the location of each cut AA1 in the tube, which may enable the tube 10K to fold at this location. As shown in FIG. 16C, the Nitinol tube 10K may then be heat-set at high temperatures or otherwise processed such that the tube 10k takes on the folded shape shown. In this manner, an elongated Nitinol tube 10K may have a plurality of spaced-apart cuts AA1 formed in the tube 10K, which allow the tube 10K to be folded into the designed orientation, and then heat set. In other examples, the cuts AA1 are designed to enable the Nitinol tube 10K to fold, bend, translate, rotate, spin, or various other movements to enable various complex orientations from the original elongated tube 10K.

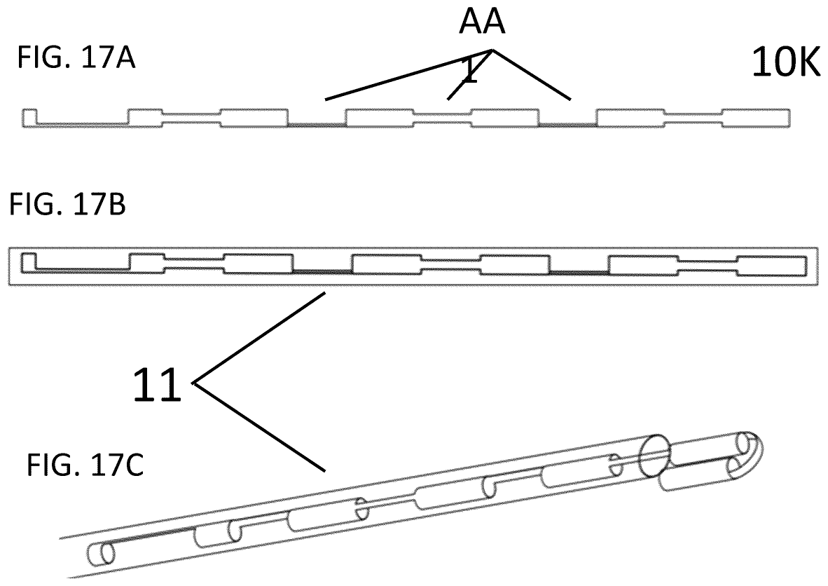

After the Nitinol tube 10K has been created and processed to take on the desired shape, the tube 10K may then be filled with a plurality of batteries, subunits, sensors, and/or processors, e.g., including components similar to other devices herein, in order to use the Nitinol tube 10K as a delivery device for the pacemaker device 8K. In other examples, the foldable Nitinol tube 10K may be used to deliver other medical devices into the body. Turning to FIGS. 17A-17C, another example of an elongated nitinol tube 10K is shown including various cuts AA1 formed in the wall of the tube. FIG. 17B shows the elongated Nitinol tube 10K introduced within a delivery sheath 11. As shown in FIG. 17C, as the Nitinol tube 10K is advanced out of the delivery sheath 11, the tube 10K automatically folds at the locations formed to cause the device to rotate, bend, spin, or otherwise translate into a desired shape within the heart or other region within the body.

In one example, the delivery sheath 11 is placed inside another larger/outer delivery sheath (not shown). For example, the system may include is a larger delivery sheath (not shown) that is used to obtain access to the left atrium. Then, the inner delivery sheath 11 may be advanced into the outer delivery sheath (not shown). In some examples, the inner delivery sheath 11 is longer than the outer delivery sheath (not shown), such that the distal end of the inner delivery sheath 11 extends beyond the distal end of the outer delivery sheath when fully advanced (not shown). Optionally, in this example, a distal tip of the inner delivery sheath 11 may include a bend or rotation formed therein that enables the distal tip of the inner delivery sheath 11 to have a predetermined orientation relative to the left atrial appendage when advanced from the larger delivery sheath. In other examples, the inner delivery sheath 11 may include a bend or curve having sufficient bias such that the inner delivery sheath 11 may cause the outer delivery sheath to bend or curve in a desired manner when the inner delivery sheath 11 is advanced into of the outer delivery sheath (not shown), e.g., to orient the sheaths within the left atrium of a patient's heart.

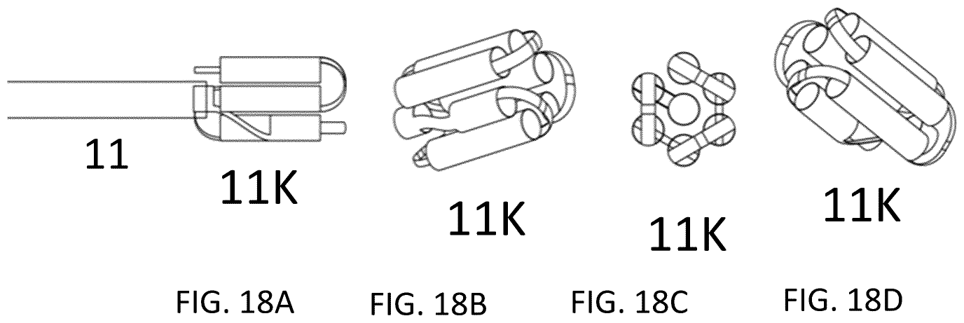

Turning to FIGS. 18A-18D, another pacing device is shown that includes a Nitinol tube 10K folded into a complex configuration 11K. In one example, the tube 10K may be configured such that one of the subunits of the elongated Nitinol tube 10K curves into the center of the folded configuration 11K, e.g., when the tube 10K is deployed from a delivery sheath (not shown). The center subunit may be hollow to allow wires and other structures (not shown) to pass through the center of the folded structure 11K. For example, this center subunit may be used to pass a guiding structure into the left atrial appendage, which may then be used as a guide or rail to advance the folded structure 11K into the left atrial appendage, e.g., after being deployed within the left atrium (not shown). This hollow center space may also be used to place a separate structure to expand or enlarge the folded structure 11K.

In some examples, the uncut nitinol tube sections may be biased to be oriented substantially parallel to one another in the folded configuration. In other examples, the folded structure 11K is angled such that the distal ends of the subunits are more compressed or closer together than their proximal ends. For example, in the deployed configuration, the distal ends of the subunits may contact one another while the proximal ends may be spaced apart, e.g., such that the folded structure 11K is biased to assume a funnel-type shape, which may facilitate advancing the device deep into the LAA.

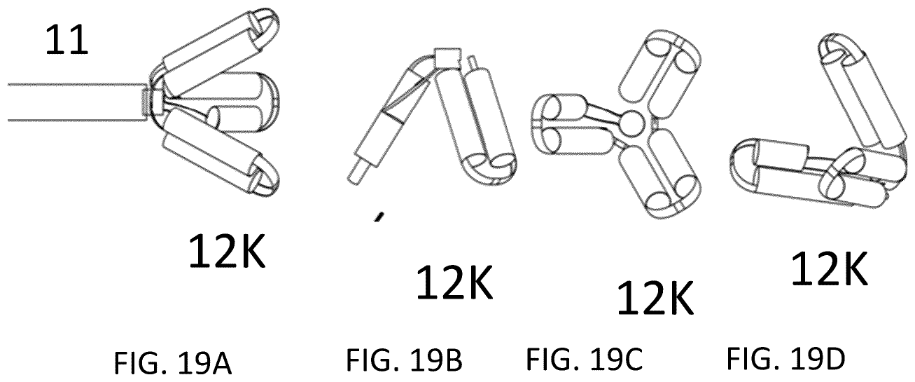

Turning to FIGS. 19A-19D, still another example of a deployed pacing device is shown that includes a Nitinol tube 10K that has folded into a folded structure 11K and is now expanded on the distal end of the folded structure 11K to an expanded structure 12K. In the example shown, the distal ends of subunits carried on the tube have expanded relative to their proximal ends. Alternatively, the proximal ends may be expanded to a larger profile than the distal ends, or the entire structure 12K may expand.

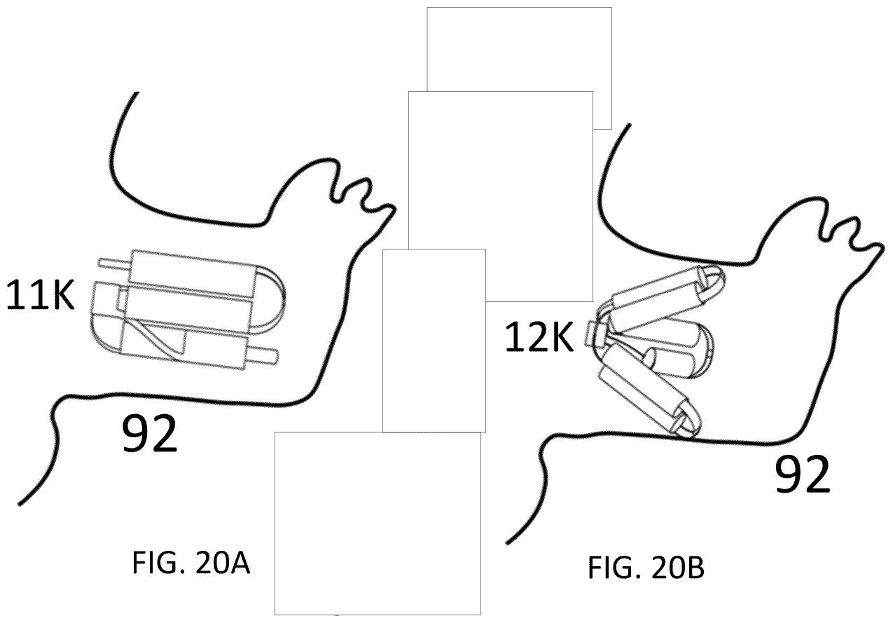

Turning to FIGS. 20A and 20B, a deployed pacing device is shown, e.g., similar to the device shown in FIGS. 19A-19D, that includes a Nitinol tube carrying subunits that has adopted a folded structure 11K and is being implanted into a LAA 92, e.g., using methods similar to other devices herein. FIG. 20A shows the folded structure 11K in a uniform configuration, while FIG. 20B shows the folded structure after being expanded into the expanded structure 12K. This expanded structure may be used to anchor the device into the LAA 92. Optionally, one or more sensors may be carried on the device, e.g., pressure sensors mounted on the expanded distal ends of one or more of the subunits. These sensors may be used to verify device positioning and adequate anchoring of the device. Optionally, the subunits may also include one or more tines, screws, or other features (not shown) to help the device anchor into the LAA 92.

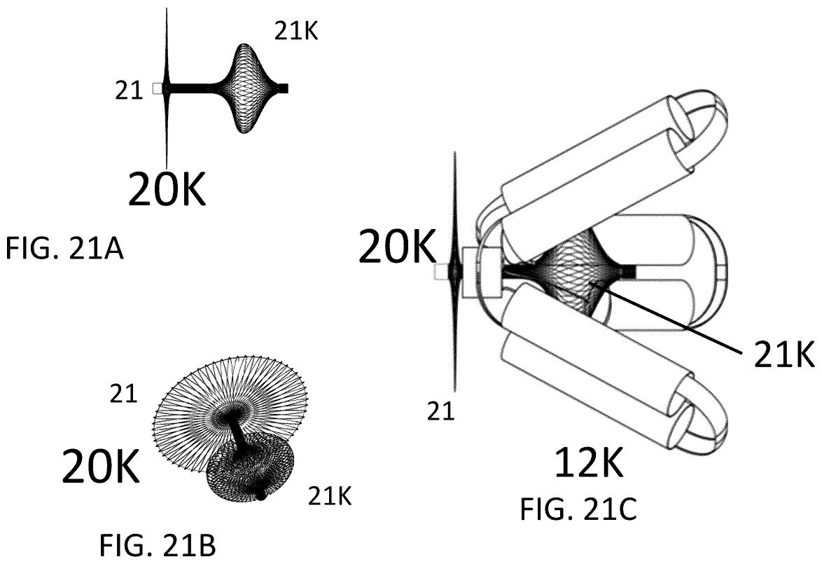

Turning to FIGS. 21A-21C, another example of a pacing device is shown that includes an enlarging component 20K that may be deployed and implanted within a left atrial appendage. In one example, the enlarging component 20K may include a cover portion 21 and an enlarging portion 21K. The cover portion 21 may be used to close off the LAA (not shown), e.g., similar to other devices herein. In some examples, the enlarging component 20K is made of braided Nitinol. In some examples, a spring or other biasing mechanism is located between the enlarging portion 21K and the cover portion 21. This spring mechanism may be used to keep the expanded structure 12K in tension with the cover 21 when deployed. For example, this tension may be used to keep the cap 21 from being pushed into the ostium of the LAA 92. In addition, this tension may be used to keep one or more anchors on the device (not shown) engaged in a predictable fashion against the tissue of the LAA. The cap 21 may be rigidly affixed to the enlarging portion 21K. In some examples, the cap is attached to the enlarging portion in such a way that the cap can rotate freely and independently of the enlarging portion and the anchoring device.

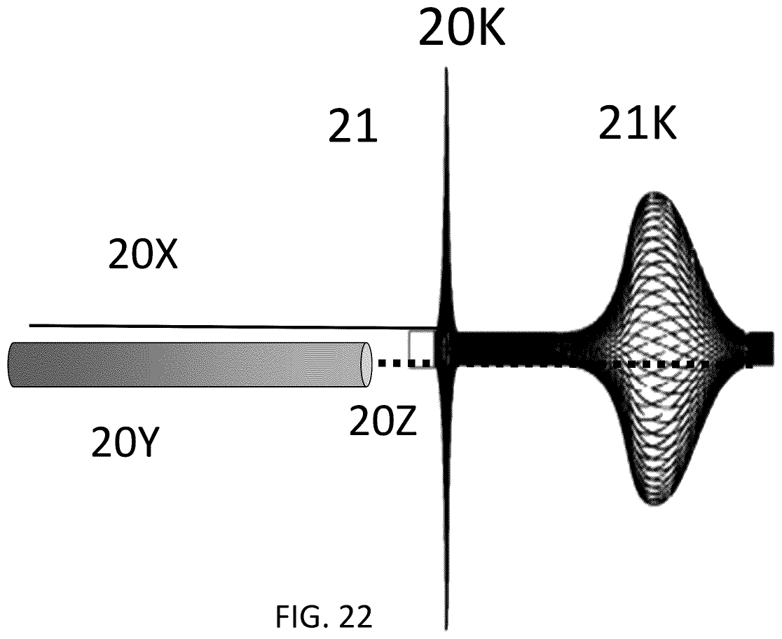

Turning to FIG. 22, another example of a distal portion of a pacing device is shown that includes an enlarging component 20K with both an enlarging portion 21K and a cover portion 21. In one example, the enlarging component 20K has multiple connections, such as 20X, 20Y, and 20Z. For example, connection 20X is a connection wire whereby the enlarging component 20K may be controlled; e.g., the connection wire 20X may be advanced or withdrawn with significant force to control the position of the entire device. Optionally, this connection wire 20X may be used to collapse the cover 21 and pull the enlarging component 20K back into a delivery sheath, e.g., after deployment (not shown). In addition, this connection wire 20X may be released to deploy the enlarging component 20K into the LAA. The releasing mechanism may be one or a combination of various mechanisms, including but not limited to spinning a screw, disconnecting, or breaking the distal end. Breaking the distal end may include delivering energy to burn or cut the distal end. In some examples, a magnetic force may be turned off to release the enlarging component 20K.

In some examples, the connection wire 20X may be more of a connection tube 20Y. In some examples, both a connection wire 20X and a connection tube 20Y may be utilized. In some examples, the connection tube 20Y may have a turning wire 20Z. The turning wire 20Z may be used to control the size of the enlarging potion 21K or the cover 21. For example, the turning wire 20Z may turn a screw (not shown). Optionally, turning the screw may be used to enlarge the enlarging portion 21K. For example, a screw and follow mechanism may be provided to controllably enlarge or collapse the enlarging portion 21K. Optionally, combined with pressure sensors on the anchors (not shown), turning the turning wire 20Z may be used to anchor the device with reliable and safe force within the LAA. Optionally, the turning wire 20Z may include an inflatable balloon (not shown) that may be inflated in order to have control turning a screw mechanism. In another option, a mechanism may be provided to control the size of the enlarging portion 21K and/or the cover 21, i.e., to allow them to be enlarged and/or collapsed as desired.

Optionally, a mechanism may be provided to release the turning wire 20Z, if included.

For example, in some cases, the turning wire 20Z may be advanced and pulled back into the enlarging portion 21K as needed. Optionally, a connector (not shown) may be provided that accepts the wire in order to turn a screw. In other examples, the turning wire 20Z has its own anchor and release mechanisms. In other examples, the turning wire 20Z may be advanced and pulled back freely, with a separate mechanism (e.g., the connection tube 20Y) used to release control of the enlarging portion 21K and/or cover 21.

In some examples, the device may include a mechanism that controls the size of the enlarging portion 21K. For example, a cover or inner brace (not shown) may be provided that may be set prior to insertion into the body. For example, mapping of the LAA may be utilized to determine the optimal size of the enlarging potion 21K before introducing the device into a patient's body. For example, measurements may be made ahead of time, and then the enlarging portion 21K may be set to the correct size. A covering tube or inner brace (not shown) may be placed or positioned so that once free, the expandable portion 21K expands to a predictable size.

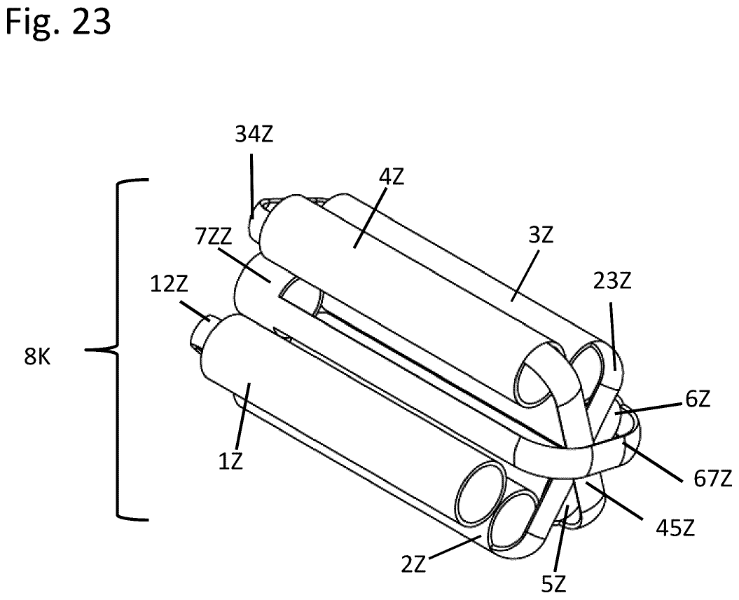

Turning to FIG. 23, another example of a device 8K is shown, i.e. an implantable pacemaker device that includes a housing having a proximal end and a distal end, the housing sized for implantation within the left atrial appendage of a heart (not shown); a cover portion (not shown), e.g., similar to other devices herein, configured to prevent thrombus from within the left atrial appendage of the left atrium to embolize out of the left atrial appendage of the left atrium; at least one pacing electrode capable of pacing cardiac tissue, e.g., adjacent the atrium; a control processor capable of processing data; and a flexible battery.

The flexible battery may be configured to change shape within the left atrial of the heart (not shown), e.g., from a contracted or delivery configuration to an expanded or deployed configuration, before being advanced into the left atrial appendage, e.g., similar to other devices herein.

In another example, the flexible battery may be configured to change shape once introduced into the left atrial appendage. In these examples, the flexible battery may change shape from an elongated shape to a more contracted shape. In another example, the flexible battery may spiral or fold inside the left atrial appendage.

Moving to the particulars of FIG. 23, the flexible battery includes a plurality of elongated sections 1Z-6Z with more flexible sections 12Z, 23Z, 34Z, 45Z, 56Z, and 67Z, e.g., connecting the elongated sections 1Z-6Z sequentially. The sections of the flexible battery are configured to transition from an elongated arrangement, e.g., a linear configuration similar to other devices herein (not shown), to a more contracted arrangement, e.g., as shown in FIG. 23. Note, the flexible battery may connect segments on the far side of the device on the distal side. While on the proximal side, the more flexible battery portions may connect segments that are adjacent. In this example, the distal end is held more tightly together, while the proximal end is configured to expand further than the distal end in the deployed configuration. For example, by placing an expanding mechanism on the inside of the device, the proximal end may expand. This expansion may be used to anchor the device 8K into the left atrial appendage.

As shown in FIG. 23, the device includes a hollow subunit 7ZZ coupled to the flexible battery. The hollow subunit 7ZZ includes a lumen sized to receive a guide wire (not shown). For example, a guide wire may be introduced into the heart, e.g., into the left atrial appendage, and used to guide the contracted device or the deliver sheath into the left atrial appendage.

In FIG. 23, the device includes three segments that expand radially to anchor the device 8K into the left atrial appendage. Some aspects of the device that contact atrial tissue may deliver electrocautery or electrical energy at the interface between the tissue and the device. The heat generated may facilitate attachment of the device to the tissue. In addition, the heat generated may facilitate blood coagulation to prevent bleeding and/or pericardial effusions. Therefore, in one example, the pacemaker device may include a housing having a proximal end and a distal end, the housing sized for implantation within the left atrial appendage of a heart; a cover portion (not shown) designed to prevent thrombus from within the left atrial appendage of the left atrium to embolize out of the left atrial appendage of the left atrium; at least one pacing electrode capable of pacing the atrium; a control processor capable of processing data; a battery; and at least one anchoring portion configured to contact cardiac tissue, wherein the anchoring portion is able to deliver electrical energy to facilitate at least one of the following: hemostasis and device adherence to the cardiac tissue. In one example, the anchoring portion may include bipolar electrodes or a monopolar electrode with a grounding pad. Delivery of electrical energy may be controlled by a feedback loop, for example, using standard electrosurgery techniques including changes in temperature, impedance, and/or power delivery, etc.

To anchor the device 8K into the left atrial appendage, the device 8K may include at least three components (for example, the components of 12Z, 34Z, and a third component (not shown), which may be configured to expand outward to anchor the device 8K by the outward radial force.

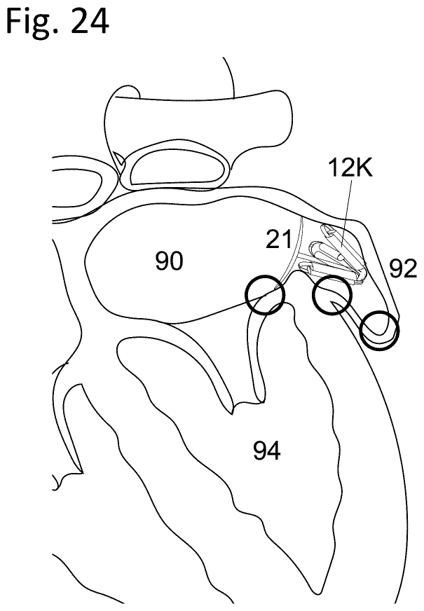

In FIG. 24, different capture locations are shown for pacing from the LAA 92. The device may capture outside the ostium in the left atrium 90, inside the LAA 92 adjacent to the left ventricle 94, or distally inside the LAA 92, as examples.

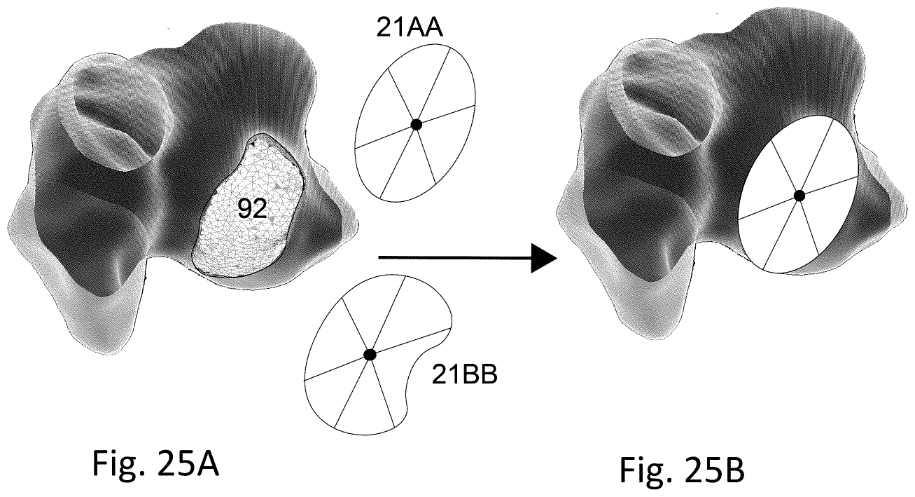

FIG. 25A shows an exemplary ostium of the LAA 92, which is typically non-circular in shape, like an oval, and a corresponding non-circular cover portion, which is used to seal the LAA 92 in FIG. 25B. Creating a non-circular cover portion may prevent leaks from forming around the closure. The cover portion may be allowed to rotate freely to achieve the best alignment during implantation. As examples of non-circular cover portion shapes, 21AA is an oval shape whereas 21BB is a symmetric “bean” shape to avoid the mitral valve. The cover portion may require rotation to optimally close the left atrial appendage. Therefore, the optimal cover portion can be a non-circular but symmetric design that closes off the left atrial appendage without impeding the pulmonary veins or the mitral valve. In some examples, the anchoring portion can connect to various cover portions. Therefore, the cover portion may be specifically designed to the shape of the patient's left atrial appendage and ostium.

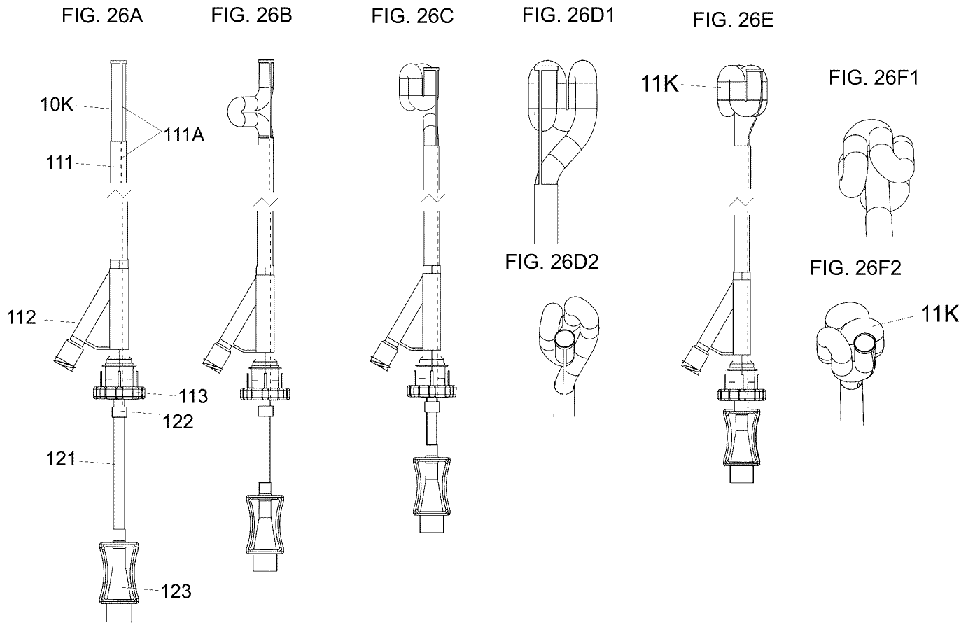

FIGS. 26A-26F show another example of a device 10K with a multi-part sheath and anchoring system. This system may be deployed through an outer sheath 11 (not pictured), e.g., similar to other devices herein. The inner sheath 111 is preloaded with device 10K. Anchoring mechanism 111A is attached to the distal end (furthest from handle) of the device 10K and runs through the length of sheath 111. The anchoring device is controllable with a mechanism 122 which can lock the anchor device in place, rotate it, or otherwise manipulate or configure the distal end of device 10K. Inner member 121 is used to push device 10K out of sheath 111 and may be controlled by mechanism 123 for manipulation of the device. Sheath 111 includes a flush port 112 to eliminate air bubbles in addition to a control mechanism 113 which can rotate, manipulate, and/or lock sheath 111 or lock inner member 121 in place with respect to sheath 111. Control mechanisms 113, 122, and/or 123 may also be locked with respect to each other in combination of independently.

FIG. 26A shows where the device 10K is fully loaded in the sheath 111 and not yet deployed. In FIG. 26B, member 121 begins pushing the device 10K out of sheath 111 which is also locked on the distal end by anchor 111A. FIG. 26C shows further deployment of the device 10K, also shown in FIGS. 26D1 and 26D2 from different angles. In FIG. 26E, the device is fully extended into its next configuration state 11K. FIGS. 26F1 and 26F2 show alternative viewing angles of the deployed device 11K.

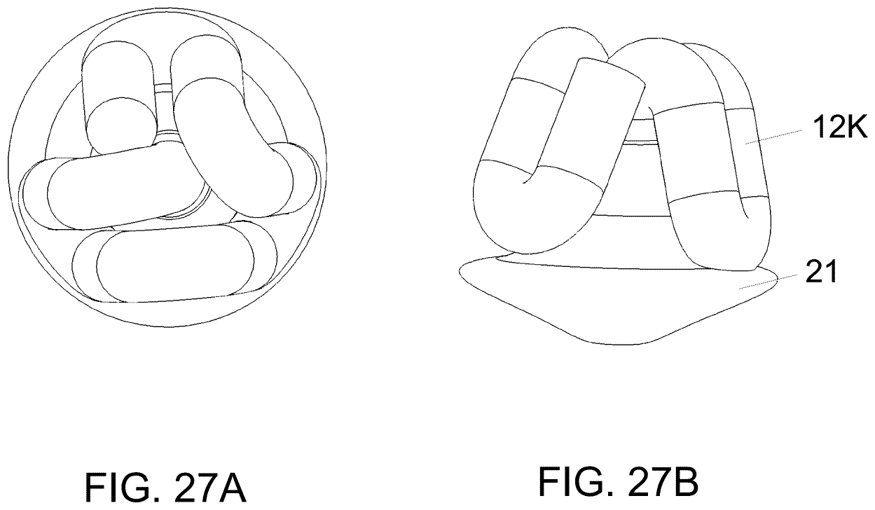

FIGS. 27A and 27B show another example (from two different viewing angles) of a device 12K which has reached a new configuration state (from straightened device 10K within the sheath, to deployed out of sheath device 11K). In this example, a combined expansion mechanism and cover portion 21 pushes against the inner walls of the device in configuration 11K to expand the device into configuration 12K. Cover portion 21 in this example is configured into a “bell” or “mushroom” shape which extends over the edges of device 12K and acts as a cap or cover to the left atrial appendage.

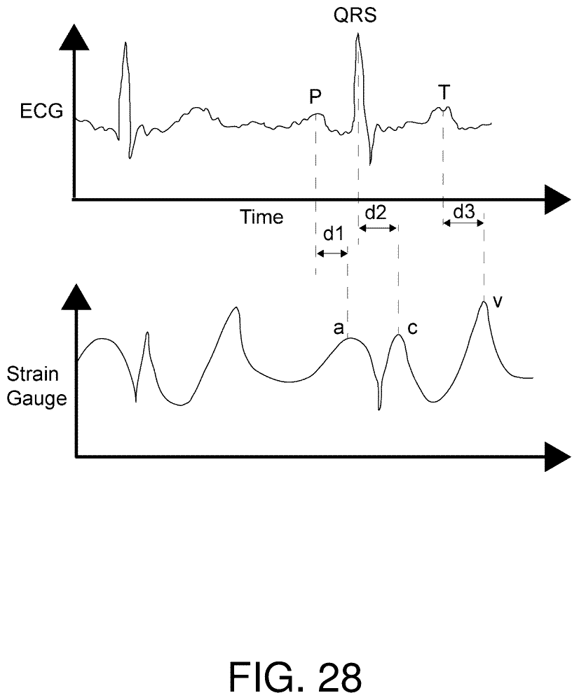

FIG. 28 illustrates a method for estimating left atrial pressure (LAP) by comparing electrical signals from an electrocardiogram (ECG) with mechanical responses detected by a strain gauge positioned on a device within the left atrial appendage (LAA). The figure is divided into two parts: the top graph represents the ECG signal, while the bottom graph displays the corresponding mechanical signal detected by the strain gauge. Top Graph-ECG Signal: The ECG graph captures the electrical activity of the heart, showing the key P, QRS, and T waves that mark distinct phases in the cardiac cycle: The P-wave indicates atrial depolarization, the electrical event that triggers atrial contraction. The QRS complex represents ventricular depolarization, initiating ventricular contraction. The T-wave corresponds to ventricular repolarization, as the ventricles relax. Bottom Graph-Strain Gauge Signal: The lower graph represents the mechanical response of the left atrium as detected by a strain gauge positioned in the LAA. This mechanical signal displays three characteristic waves: The a wave represents atrial contraction that occurs following the P-wave. The c wave reflects the ventricular contraction impact as the mitral valve bulges into the left atrium during ventricular contraction. The v wave signifies venous filling of the left atrium as blood flows into it during ventricular relaxation.

Time Delay Measurement (d1, d2, d3): The time intervals d1, d2, and d3 represent the delays between specific ECG signals and their corresponding mechanical responses: d1 is the time delay between the P-wave (atrial depolarization) and the a wave (atrial contraction). d2 measures the delay between the QRS complex (ventricular depolarization) and the c wave (ventricular contraction impact). d3 is the delay between the T-wave (ventricular repolarization) and the v wave (venous filling). Use of Time Delays for Estimating Left Atrial Pressure: The system leverages these time delays (d1, d2, d3) to estimate left atrial pressure dynamically. The underlying principle is that changes in left atrial pressure affect the compliance of the atrial wall, thereby altering the time delays between electrical events (ECG) and mechanical responses. For instance, an elevated left atrial pressure results in reduced compliance, causing shorter time delays as the atrial wall stiffens. Conversely, a lower atrial pressure leads to longer delays due to increased compliance. By continuously monitoring and analyzing these delays, the system provides a real-time, non-invasive estimation of LAP. This method offers an innovative approach to cardiac monitoring by using time delay analysis rather than direct pressure measurements, making it a minimally invasive solution for patients with cardiovascular conditions.

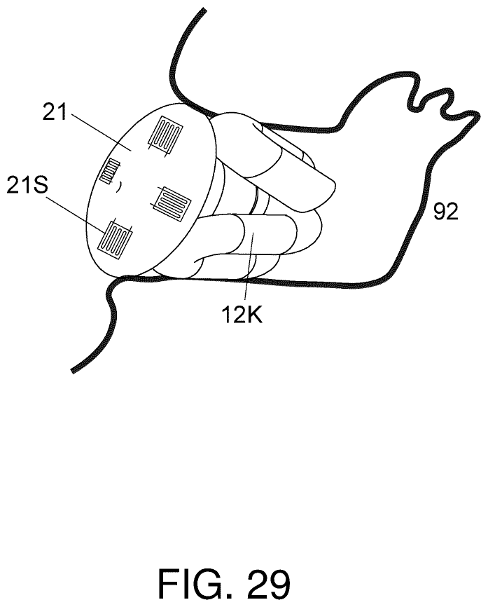

FIG. 29 is an example of device 12K embedded within the left atrial appendage 92, which has a cover portion 21 that faces into the left atrium. Embedded within the cover portion 21 are strain gauge sensors 21S, which are specifically configured to detect mechanical deformations in response to pressure changes within the left atrium. The cover portion's location at the ostium enables it to monitor the mechanical forces exerted by atrial wall movements during different phases of the cardiac cycle. Strain gauge sensors 21S are sensitive to changes in tension and compression that occur as the left atrium contracts and relaxes. By measuring these mechanical responses, the sensors provide real-time data on atrial wall compliance, which is directly related to left atrial pressure (LAP). The strain gauges within cover portion 21 detect subtle changes in the atrial wall's mechanical behavior, which can be correlated to pressure fluctuations. For example, during atrial contraction, an increase in atrial pressure leads to measurable deformation in the cover portion. This deformation is captured as a strain signal by sensors 21S, enabling the device to detect atrial pressure changes without a direct pressure sensor. The strategic placement of the strain gauge sensors at the ostium ensures that the sensors detect pressure-related deformations in a consistent manner, as this region is exposed to both atrial contractions and ventricular impacts (e.g., from the mitral valve during ventricular systole). The data collected by the strain gauge sensors 21S is transmitted to a data acquisition module within or connected to device 12K, where it can be synchronized with electrocardiogram (ECG) signals or other mechanical signals from the heart. In certain examples, the device may also include wireless communication capabilities, allowing it to send the strain gauge data to an external processing unit or clinician's interface for further analysis, enabling remote monitoring of left atrial pressure.

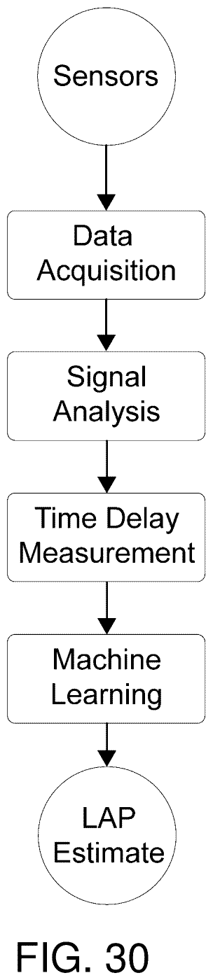

FIG. 30 shows an exemplary flow chart explaining the process for reading sensor data and estimating LAP, e.g., using one or more processors included in any of the devices and systems described herein. FIG. 30 provides a simple block diagram outlining the process for reading sensor data and estimating left atrial pressure (LAP) in a sequential flow. The diagram illustrates the core stages in the data processing pipeline, from data collection to LAP estimation.

The process begins with Sensors, which represent the collection of data from various sources, such as ECG, strain gauges, and accelerometers embedded in or near the left atrial appendage, e.g., as described in the exemplary devices and systems described elsewhere herein. These sensors capture both electrical signals (e.g., P, QRS, and T waves from the ECG) and mechanical responses (e.g., a, c, and v waves) that correlate with the heart's physical activity.

The data collected by the sensors is transmitted to the Data Acquisition block, where it is synchronized and prepared for analysis. This stage ensures that the ECG and mechanical signals are aligned in time, enabling accurate comparison and correlation between the electrical and mechanical events within the cardiac cycle.

In the Signal Analysis block, the processor(s) processes the acquired data to extract essential features, such as signal amplitude and waveform shape, that provide insight into the mechanical behavior of the left atrium. This step prepares the data for the Time Delay Measurement, where the system calculates the time intervals between specific ECG signals (e.g., P-wave) and corresponding mechanical responses (e.g., a wave). These time delays are critical for estimating LAP, as variations in delay can indicate changes in atrial pressure and compliance. The Machine Learning block utilizes the extracted features and measured time delays as inputs to a trained model. This model, developed using datasets with known LAP measurements, analyzes patterns within the data to predict the current left atrial pressure. Finally, the LAP Estimate is provided as an output, representing the real-time assessment of left atrial pressure. This estimate can be displayed on a clinician's interface or used to inform further cardiac monitoring and treatment decisions. This sequential flow enables a continuous, non-invasive method for estimating LAP based on synchronized electrical and mechanical signals from the heart.

In some examples, the device for implanting within the left atrial appendage (LAA) of the heart is designed for adaptive deployment through a dual-sheath delivery system. The deployment process begins by inserting an outer sheath, which serves as a guiding structure, into the left atrium of the heart. The device itself is preloaded into an inner sheath, which is carefully flushed with saline to eliminate air bubbles and ensure smooth delivery.

An anchor mechanism, e.g., in the form of a wire or similar structure, is attached to the distal end of the device. This anchor is user-actuated from the proximal end, enabling the physician to hold the distal end steady as the device is deployed. As the device is pushed out of the inner sheath, the anchor secures the distal end, allowing the various components of the device to unfurl and adopt their preset shape around the first subunit. This process ensures a stable and controlled deployment within the left atrium.

Once the device has been fully extended and configured outside of the inner sheath in the left atrium, the outer sheath is then used to guide the entire assembly into the LAA. This dual-sheath method allows for gradual and precise advancement, minimizing the risk of damaging the delicate heart structures. When the device is correctly positioned within the LAA, the physician can actuate an expander mechanism that pushes the configured device outward, ensuring optimal contact with the LAA walls. This expander can take various forms, such as a mesh cage, a balloon, or other expandable structures, each designed to create an even distribution of force against the LAA walls for secure positioning.

The expander may also facilitate the deployment of a cover element, which balloons out from the proximal end of the device or otherwise expands within the LAA or left atrium to form a cap over the opening of the LAA. In one example, this cover may adopt a mushroom or bell-like shape to provide effective isolation of the LAA from the left atrium. Alternatively, the cover may be an independent component from the expander, configured to extend and seal the LAA ostium once the device is securely anchored. The flexibility in design ensures that different LAA morphologies can be effectively managed.

Before advancing the device into the LAA, the physician may also release the anchor. This can be done through either mechanical or electrical methods, enabling the device to maintain its position autonomously while adapting to the anatomical structure of the LAA. The anchor-release feature ensures that, once in place, the device is free to conform to the unique contours of the LAA, while maintaining its stability.

In other examples, the anchor is released after the device is implanted in the LAA, and is capable of translating and/or rotating independently of the sheath, to be used as an additional steering mechanism to place the device correctly in the appendage.

The combination of the dual-sheath delivery system, anchoring mechanism, expander, and cover component provides a comprehensive solution for securely and effectively isolating the LAA. These features ensure precise placement and long-term stability, significantly reducing the risk of thrombus formation and improving patient outcomes in atrial fibrillation treatment.

In addition to having electrodes coupled to the atrial side of the cover portion, optionally, the cover portion may also have tines or anchors to help anchor the cover to the atrial tissue. In some examples, the tines have a certain orientation, such that by rotating the cover portion, the tines are driven into the atrial tissue in order to anchor the cover portion into the atrial tissue. In addition, this system or method may be used to drive the pacing electrodes into the atrial tissue. In some examples, the electrodes may be needle-like. The rotational force may be used to drive the needle-like electrodes into the atrial tissue. This can be used to anchor the cover portion as well as obtain optimal sensing and pacing parameters for the electrodes.

In addition or alternatively, electrical energy may be delivered to the electrodes to heat the electrodes. This electrical energy or heat may be used to facilitate device adherence to the tissue. In another example, this electrical energy or heat may be used to obtain hemostasis, or stop any bleeding at or near the location of the electrode. In another example, this electrical energy or heart may be used to change the electrical impedance of the electrode.

In other examples, the device may include at least one electrode coupled to the cover portion. The electrode is designed to be placed adjacent the cover potion and the left atrial tissue; but outside of the left atrial appendage. In some examples, the device may include at least two electrodes, coupled to the cover portion; and positioned outside the left atrial appendage, but adjacent atrial tissue. The cover portion is designed to cover the electrode so this electrode is not in direct contact with free-flowing blood flow within the left atrium.

In some cases, the cover portion may be positioned over the left atrial ostium; however, the pacing electrodes may not have optimal sensing or capture threshold parameters. Therefore, the cover portion may be rotated in order to obtain better sensing or pacing parameters. In addition, the cover portion may not deliver enough force towards the left atrium such that the pacing electrodes are optimally adjacent atrial tissue. Therefore, the cover portion may be advanced towards the anchoring portion; such that the cover portion is tightened adjacent the ostium of the left atrial appendage. The advancement of the cover portion to the anchoring portion may reduce the slack between these two portions. This may be achieved through various mechanisms including turning a screw, shortening a tether, among various other potential mechanisms.

Therefore, in one example, a leadless pacemaker device may be provided that is designed for implantation within or near a left atrial appendage extending from a left atrium of a heart to monitor and/or treat a patient with conduction abnormalities and/or cardiac dysrhythmias, the device including a battery capable of storing electrical energy; an anchoring portion to anchor the device within the left atrial appendage; at least one electrode configured for sensing and pacing the left atrium; a control processor configured for processing data; a communication module to communicate to other devices; and a cover portion configured to prevent thrombus from within the left atrial appendage of the left atrium to embolize out of the left atrial appendage of the left atrium; wherein the cover portion is configured to be rotated independently of the anchoring portion. In some examples, there may be at least one electrode coupled to the cover portion but located outside the left atrial appendage. In other examples, there are at least two electrodes, coupled to the cover portion, adjacent to atrial tissue, and located outside the left atrial appendage.

In another example (not shown), the flexible battery may be configured to spiral within left atrial appendage during deployment. In one example, the flexible battery may be configured to assume a tight spiral, e.g., in a delivery condition, which may then expand into a wider spiral, e.g., when deployed within the patient's heart. The expansion of the spiral may hold the flexible battery within the left atrial appendage. In another example, the flexible battery may be configured to assume a relatively tight spiral or coil in the delivery condition, and the tight spiral may be configured to at least partially unravel to expand the flexible battery within the left atrial appendage. In other examples, the flexible battery may be biased to expand, e.g., to spiral outwardly automatically, when the device is deployed from a delivery sheath, e.g., to substantially to fill the left atrial appendage with more battery than if the flexible battery advanced straight into the left atrium appendage.

In some examples, the device may physically occlude the opening of the LAA while incorporating electronic components for pacing, sensing, or therapeutic functions. For example, the device may include a self-expanding metal frame made from materials, such as nitinol or other shape-memory alloys, covered with a permeable or impermeable membrane. Upon deployment, the frame may expand to engage the walls of the LAA securely. Electronic components such as sensors, electrodes, and power sources may be integrated into the frame or membrane. The membrane may serve both to isolate the LAA and as a platform for electronic integration.

In some examples, the device may include a collapsible framework that expands upon deployment to conform snugly to the LAA's internal anatomy, providing a stable platform for electronic components. Constructed from flexible materials, the device may be constrained during catheter delivery and expand when released into the LAA. For example, the device may include anchoring elements like radial force mechanisms or flexible struts that press against the LAA walls, ensuring consistent contact and secure anchoring, accommodating various LAA sizes and shapes. In addition or alternatively, the device may also include a helical or corkscrew shape that anchors by being gently screwed into the LAA tissue. Delivered via a rotational catheter system, the device may be rotated during deployment to embed its helical structure into the LAA wall. The central shaft may house electronic components, and the helical threads may act as both the anchoring mechanism and the electrical interface with cardiac tissue.

In some examples, the device may open like an umbrella or disc within the LAA to provide occlusion and anchoring. Delivered in a collapsed state, the device's canopy may expand upon deployment to cover the LAA orifice. Peripheral anchors or hooks may secure it to the LAA walls. The umbrella surface may be embedded with sensors, electrodes, or energy-harvesting components, ensuring even distribution of force and minimizing pressure points on the tissue. A plug-shaped device may feature expandable lobes or flanges that anchor into the LAA. Inserted into the LAA opening, the device's lobes may expand outward to press against the walls, securing it in place. The central body may contain the electronics and power source. The plug may serve the dual purpose of anchoring the device and occluding the LAA to prevent blood flow into the appendage.

In some examples, the device may also be a mesh structure that expands to fill the LAA, providing anchoring and a surface area for electronic integration. Made from self-expanding materials, the device may be compressed during delivery and expand within the LAA. The mesh may conform to the appendage's internal contours, and its struts may incorporate electrodes and sensors. It may exert gentle radial force to remain in place without damaging the tissue. The device may feature biomimetic designs that mimic biological anchoring mechanisms, such as tendrils or barbed structures, to enhance integration with cardiac tissue. Featuring micro-scale barbs, hooks, or Velcro-like surfaces, the device may engage with the natural trabeculations of the LAA. These structures may provide secure anchoring while minimizing trauma and promoting endothelialization, potentially reducing the risk of thrombosis.

In another example, the device may include bioadhesive elements that bond to the left atrial appendage (LAA) tissue upon exposure to blood or other specific biochemical cues. The bioadhesive materials may include reactive polymers that bond upon interaction with the LAA's unique chemical environment. For instance, a pH-sensitive adhesive may activate to secure the device upon contact with the blood, eliminating the need for tines, screws, or mechanical expansion. This anchoring method allows for stable device fixation through bio-compatible chemical bonding, ensuring effective implantation without mechanical protrusions or anchoring structures.

In another example, the device may use photothermal attachment to bond with LAA tissue without relying on mechanical components. The anchoring process uses a low-energy, localized light source, such as a laser or LED, to induce mild thermal changes in specific points on the device's surface, causing limited tissue adhesion. The photothermal effect allows the device to secure itself to the LAA with precision, creating a stable bond through targeted heat without requiring tines, radial forces, or additional invasive components. This technique provides a minimally invasive, controlled anchoring solution suitable for sensitive atrial tissue.

In some examples, the device may use biocompatible adhesives to bond with the LAA tissue. Upon deployment, the device may apply a medical-grade adhesive to its contact surfaces, creating a strong bond with the endocardial tissue. This method may avoid mechanical fixation, reducing the risk of tissue perforation or damage. The device may also anchor by inflating a balloon within the LAA to fill the space and press against the walls. It may include a deflated balloon during delivery, which may be inflated with a biocompatible fluid or gas upon placement. The balloon may conform to the LAA shape, providing stable anchoring. Its surface may be embedded with electrodes or sensors and may include a self-sealing mechanism to maintain inflation.

In some examples, the device may use mechanical anchors in conjunction with membranes to both secure and isolate the LAA. It may deploy mechanical anchors, such as hooks or expandable frames, and include a membrane or mesh that covers the LAA entrance. The membrane may provide a barrier to blood flow, aiding in thrombosis prevention, while the anchors ensure device stability. The device may also anchor by attaching sutures to the LAA tissue. Specialized delivery tools may place sutures through the device and into the LAA wall. Once secured, the sutures may be tied or fixed with clips, anchoring the device. This method may allow for precise positioning and adjustment during the procedure.

In some examples, the device may use magnetic attraction to anchor within the LAA. For example, the device may include magnetic elements that interact with magnetized particles delivered to or coated on the LAA tissue. Magnetic forces may hold the device in place without mechanical penetration, potentially reducing tissue trauma. The device may be equipped with claw-like appendages that grip the LAA tissue upon deployment. The claws may be folded during delivery and extend outward when the device reaches the LAA. They may gently grasp the tissue, providing secure anchoring with atraumatic tips to minimize injury.

In some examples, the device may utilize hydrogels that expand upon absorbing bodily fluids to anchor within the LAA. Components made of hydrogel materials may swell after exposure to blood, filling the LAA cavity and securing the device through mechanical pressure. The hydrogels may be biocompatible and designed to degrade over time if temporary anchoring is desired. The device may also be a mesh structure with integrated barbs that anchor into the LAA tissue. Deployed within the LAA, the mesh's barbs may engage with the trabeculated inner surface, providing a scaffold for electronic components. The mesh may be customized to match the patient's anatomy for optimal fit.

In some examples, the device may include flexible tails or filaments that extend into the LAA for anchoring. The tails may become entangled within the natural trabeculations of the LAA, anchoring the device through friction and mechanical interaction without penetrating the tissue. The device may adhere to the LAA walls using suction created by negative pressure. It may include suction cups or chambers that, when activated, create a vacuum effect, causing it to adhere to the tissue. This anchoring method may avoid mechanical fixation and can be reversed if repositioning is necessary.

In some examples, the device may deploy a biocompatible foam within the LAA to provide anchoring and occlusion. Upon deployment, the device may release a foam that expands to fill the LAA. The foam may solidify, anchoring the device and isolating the LAA. Optionally, the foam may be formulated to be biodegradable if temporary occlusion is desired.

Optionally, any of the devices herein may include multiple parts that assemble and lock together within the LAA. Delivered sequentially via catheter, the components may interconnect within the LAA, forming a cohesive structure. This approach may allow larger devices to be implanted through smaller catheters, providing secure anchoring through mechanical interlocking.

In some examples, the device may utilize materials that change properties in response to stimuli like temperature or pH. Made from shape-memory polymers or alloys, the device may remain flexible during delivery and become rigid upon exposure to body temperature or specific chemical environments. This change may enhance anchoring and structural integrity. The device may partition the LAA into smaller sections to prevent thrombus formation and provide anchoring surfaces. It may extend frameworks or barriers within the LAA, reducing chamber size and limiting blood stasis. These structures may anchor the device and promote tissue integration.

In some examples, the device may anchor by exerting outward radial force against the LAA walls. Similar to vascular stents, the device may expand to press uniformly against the LAA interior, providing frictional anchoring without penetrating the tissue. Thus, the device may accommodate different LAA sizes. The device may be designed with surfaces that encourage tissue growth into them, enhancing long-term anchoring. Featuring porous surfaces or bioactive coatings, the device may promote endothelial cell migration and tissue ingrowth. Over time, the device may become integrated with the cardiac tissue, providing stable anchoring and reducing dislodgement risk.

In some examples, the device may include anchoring elements that can be adjusted post-deployment to optimize positioning. For example, the device may include components that can be expanded, contracted, or repositioned using external controls or specialized catheters, allowing fine-tuning of the device's position and anchoring force after deployment. The device may also feature flexible joints that accommodate cardiac motion, reducing stress on anchoring points. Incorporating ball-and-socket or flexible joint mechanisms, the device may move with the heart's contractions, minimizing mechanical stress on both the tissue and the device, potentially increasing durability and anchoring reliability.

These mechanical configurations provide a range of strategies for designing implantable devices that anchor securely within the LAA and effectively isolate it from the left atrium. By carefully considering factors such as anatomical variability, deliverability, stability, and integration with electronic components, these designs aim to enhance patient outcomes while minimizing risks associated with implantation. Key considerations for mechanical implementation include ensuring biocompatibility, deliverability, stability, retrievability, promoting endothelialization, minimizing thrombus formation, and supporting the incorporation of electronic components without compromising mechanical function. By combining these mechanical designs with advanced electronic capabilities, implantable devices in the LAA may provide effective therapies for cardiac conditions while ensuring patient safety and device longevity.

In one example, the systems herein may include a cardiac pacing system designed to enhance atrial synchrony in patients with delayed interatrial conduction. In patients with delayed interatrial conduction, the left atrium contracts after the left ventricular contraction, resulting in asynchronous contraction that can lead to suboptimal cardiac performance and reduced hemodynamic efficiency. Traditional pacemaker systems typically focus on right atrial and ventricular pacing, which may not adequately address interatrial conduction delays. To address this issue, the invention may include a right atrial lead positioned in the right atrium and a left atrial appendage (LAA) pacing device implanted in the LAA. The right atrial lead may be configured to sense intrinsic atrial activity or deliver pacing pulses to the right atrium. The LAA device may be equipped to detect signals from the right atrial lead and, in response, deliver pacing stimuli to the left atrium after a predetermined delay. This coordination may ensure that the left atrium contracts in synchrony with or immediately after the right atrium, improving overall cardiac function.

The system may include a right atrial lead implanted in the right atrium, which may function to sense intrinsic atrial electrical activity, deliver pacing pulses when intrinsic activity is absent or inadequate, and transmit identifiable electrical signals or markers detectable by the LAA device. The pacing device may be implanted within the LAA and may be designed to detect signals from the right atrial lead, either through direct electrical sensing of myocardial activity or via wireless communication methods such as radiofrequency (RF) signals. The device may compute an appropriate delay interval after detecting the right atrial signal and deliver a pacing pulse to the left atrium to induce timely contraction.

Upon sensing a natural or paced event in the right atrium, the right atrial lead may generate a signal detectable by the LAA device. The LAA device, upon detecting this signal, may initiate a timing sequence based on a preset atrioventricular (AV) or interatrial (IA) delay. After the delay, the LAA device may deliver a pacing pulse to the left atrium, ensuring synchronized contraction with the right atrium. By coordinating left atrial pacing with right atrial activity, the system may mitigate the effects of delayed interatrial conduction, improving overall hemodynamics by contributing to more efficient ventricular filling and cardiac output. The delay intervals may be programmed to suit individual patient physiology, optimizing therapy for improved outcomes.

In some examples, the device may use a dual component pacing system, consisting of a right atrial lead and a pacing device placed within the left atrial appendage (LAA). The right atrial lead is implanted in the right atrium, where it senses intrinsic atrial electrical activity and, when necessary, delivers pacing pulses. The lead also transmits an identifiable electrical signal that is detectable by the pacing device. The device, implanted directly within the LAA, detects these signals from the right atrial lead and responds by delivering pacing stimuli to the left atrium after a calculated delay interval. This delay may be adjusted to promote synchronized contraction of both atria, improving overall cardiac function.

During typical operation, the right atrial lead senses intrinsic atrial activity and transmits a signal upon detection of atrial depolarization. The device, equipped with detection circuitry, receives this signal and initiates a countdown based on a preprogrammed delay interval. The device then delivers an electrical pacing pulse to the left atrium, ensuring that it contracts in synchrony with, or just after, the right atrium. This coordination is essential for enhancing the hemodynamic contribution of atrial contraction, especially in patients where delayed interatrial conduction impairs ventricular filling and reduces cardiac output.

The device may include features to facilitate communication between the right atrial lead and the pacing components. Detection of right atrial signals may occur through direct myocardial conduction, where the right atrium's electrical activity propagates through cardiac tissue to the device, or through wireless communication methods such as radiofrequency (RF) signaling. In examples utilizing RF signaling, the device may include a receiver capable of capturing transmissions from the right atrial lead, enabling precise timing and delivery of left atrial pacing.

The device may be programmable, allowing physicians to customize the delay interval between right atrial activity and left atrial pacing to meet individual patient needs. This customization ensures that pacing is tailored to the patient's specific conduction characteristics, thereby improving synchrony and promoting optimal atrial function. The delay interval may be adjusted over time as the patient's condition evolves, ensuring that the therapeutic benefit is maintained throughout the course of treatment. The system's dual-component design may provide significant therapeutic advantages over traditional pacing systems, which often fail to adequately address delayed atrial conduction. By enhancing atrial synchrony, the system contributes to more efficient ventricular filling, improved cardiac output, and better overall hemodynamic performance. This is particularly advantageous for patients with heart failure or other cardiac conditions where atrial contraction plays a critical role in supporting ventricular function. By timing the left atrial contraction to closely follow that of the right atrium, the invention mitigates the negative effects of delayed interatrial conduction, thus optimizing cardiac performance.

As an alternative to traditional metallic leads, the device may include optical fibers designed for optogenetics-inspired pacing. Optical fibers positioned near atrial tissue deliver light pulses to genetically modified cardiac cells that are responsive to specific light wavelengths. This approach utilizes non-metallic, flexible optical fibers that minimize interference with MRI imaging and reduce the structural invasiveness of traditional leads. Through gene therapy, cardiac cells in targeted atrial areas could be modified to respond to light-based stimulation, enabling precise and minimally invasive control of atrial rhythm. This optical pacing method provides a novel pathway for managing atrial fibrillation without conventional electrical pacing methods.

In another example, the pacing device may include epicardial pacing electrodes positioned on the outer surface of the left atrium through a subcutaneous approach. This epicardial configuration avoids direct penetration into the left atrial chamber, reducing procedural complexity and potential risks associated with endocardial placements, particularly for patients with complex atrial anatomies or prior scarring. By using minimally invasive thoracoscopic techniques to place the electrode array on the epicardial surface, the device provides effective atrial pacing while minimizing trauma to intracardiac structures. This approach also supports epicardial lead placement with broader access to atrial tissue, offering enhanced options for pacing in non-traditional atrial zones.

The pacing device may include an integrated adaptive algorithm powered by machine learning, which refines pacing intervals and settings based on historical and real-time physiological data. This AI-driven pacing algorithm analyzes patient-specific data, such as heart rate trends, arrhythmia patterns, and other physiological metrics, to predict and adjust pacing needs dynamically. The algorithm continuously learns and updates based on each patient's response to pacing, optimizing energy efficiency and reducing unnecessary pacing. By leveraging patient history and current physiological states, this adaptive approach tailors pacing delivery to the individual's unique cardiac profile, promoting enhanced therapeutic effectiveness and energy conservation.

To accommodate non-uniform atrial fibrosis patterns and evolving atrial needs, the device may further include a multi-electrode array deployed within or adjacent to the left atrial appendage. This electrode array allows selective stimulation in multiple zones within the atrium, adjusting the pacing location as necessary to address irregular atrial rhythms or scarring. Through selective zone-based pacing, the multi-electrode array can dynamically control the area of stimulation, enhancing efficacy in atria with fibrotic or heterogeneous conduction areas. The array enables flexible pacing responses tailored to various physiological conditions, supporting atrial synchrony with higher precision and adaptability.

In some examples, the device may include electrodes or energy delivery elements to facilitate ablation, providing additional therapeutic options for patients who require electrical isolation of the left atrium. In cases of atrial fibrillation or other arrhythmias, the ability to deliver ablation energy from the device allows dual-purpose treatment, combining pacing with electrical modification of the atrial substrate. This feature may be utilized in conjunction with the pacing function, using the same detection and control systems to provide a seamless therapeutic solution.

Optionally, any of the devices and systems herein may also integrate a power source, which may be in the form of a battery or an energy harvesting system that collects power from cardiac motion or other physiological activities. The device's power management system may include features to conserve energy, such as duty cycling or adaptive power usage, ensuring a long operational lifespan despite the limited space available within the LAA. Alternatively, the system may be wirelessly rechargeable, utilizing inductive coupling to recharge the internal power source as needed, thereby minimizing the need for surgical intervention to replace the battery.

To facilitate ease of implantation, both the right atrial lead and the pacing device may be designed for catheter-based delivery. The right atrial lead may be introduced through the venous system and positioned within the right atrium, while the LAA pacing device may be delivered transeptally via the left atrium. Once positioned within the LAA, the pacing device may use a combination of mechanical anchors, such as tines, hooks, or self-expanding structures, to secure itself in place. Secure anchoring ensures consistent contact with atrial tissue, which is essential for both pacing efficacy and signal detection.

The systems and methods herein may also encompass additional features and functionalities that enhance the system's versatility and therapeutic potential for managing delayed interatrial conduction. The system may incorporate advanced sensing capabilities that extend beyond simply detecting right atrial activity. The right atrial lead and pacing device may include high-resolution sensors that continuously monitor various physiological parameters, such as atrial pressure, electrical conduction velocity, and atrial wall motion. These sensors may provide valuable data that can be processed by the control unit to optimize the timing of pacing pulses, ensuring that the delay interval between right and left atrial contractions is adjusted in real-time based on the patient's physiological state. By incorporating these additional sensing capabilities, the system may provide a more responsive and adaptive approach to managing atrial synchrony, improving overall cardiac efficiency.

In some examples, the device may also include a mechanism for dynamic anchoring, allowing the device to adjust its positioning within the LAA in response to changes in cardiac anatomy or hemodynamic conditions. This dynamic anchoring may be facilitated through the use of expandable structures, such as shape-memory alloys or inflatable elements, which may be controlled by the pacing device's control unit. By enabling the device to reposition itself as needed, the risk of dislodgement or suboptimal contact with atrial tissue may be minimized, thereby enhancing both pacing efficacy and long-term device stability. In one example, the device may include an expandable framework that adjusts its shape based on real-time feedback from sensors monitoring tissue contact and anchoring stability.

The system may incorporate a dynamic adaptive anchoring mechanism designed to respond to changes in patient activity levels and hemodynamic conditions. In some examples, the anchoring mechanism may include elements that automatically adjust their force or configuration based on sensor inputs, such as variations in heart rate or atrial pressure. This adaptability may ensure that the device remains securely anchored during periods of physical exertion or other activities that alter cardiac dynamics, thereby reducing the risk of migration or inadequate contact with atrial tissue. The dynamic anchoring mechanism may utilize materials like shape-memory alloys or electroactive polymers that change shape or stiffness in response to electrical stimuli or temperature changes.

In addition to pacing and ablation, the LAA device may be equipped with drug delivery capabilities, allowing for the localized administration of therapeutic agents directly to the left atrium or LAA. This drug delivery feature may be particularly beneficial for managing inflammation, reducing the risk of thrombus formation, or delivering anti-arrhythmic medications to prevent the recurrence of atrial fibrillation. The drug delivery mechanism may be included with the pacing system's control unit, allowing for precise timing and dosage of drug release in coordination with the patient's cardiac cycle. In one example, the device may include a micro-pump that releases medication in response to detected increases in atrial pressure or other indicators of arrhythmic events.

Optionally, the systems herein may further incorporate a multi-modal energy harvesting module, designed to supplement the device's power supply by harnessing various forms of energy from the body. In some examples, the device may utilize piezoelectric elements to convert the mechanical energy generated by cardiac contractions into electrical energy, which can then be used to power the device. Additionally, the system may include thermoelectric materials that generate electricity from temperature gradients within the body, or electromagnetic induction mechanisms that capture energy from blood flow. By integrating multiple energy harvesting methods, the device may achieve a more sustainable power supply, thereby extending its operational lifespan and reducing the need for battery replacements or recharging procedures.

The device may also include patient-specific customization features to improve its fit and performance within the LAA. In one example, pre-procedural imaging data, such as CT or echocardiogram images, may be used to create a 3D model of the patient's LAA anatomy. This model may then be used to produce a customized device or components that conform precisely to the anatomical contours of the LAA, ensuring effective isolation and minimizing the risk of thromboembolic events or dislodgement. By tailoring the device to the patient's unique anatomy, the risk of complications may be reduced, and the effectiveness of the pacing and isolation functions may be enhanced.

Wireless communication capabilities may also be integrated into the systems herein to enable remote monitoring and programming of the pacing device. The device may be equipped with a low-energy communication module, such as Bluetooth Low Energy (BLE) or similar technology, allowing it to transmit data to an external receiver or a wearable monitoring device. This capability may provide healthcare providers with continuous access to real-time data regarding device performance, atrial activity, and patient-specific metrics, enabling more proactive management of the patient's condition. Additionally, the communication module may support non-invasive reprogramming of pacing parameters, allowing for adjustments to be made without the need for surgical intervention. In one example, the device may also include an emergency alert system that communicates critical data to healthcare providers in the event of significant changes in cardiac function.

The systems and methods herein may also include one or more safety features designed to prevent complications associated with pacing or ablation therapy. For example, the device may be equipped with temperature sensors that monitor tissue temperature during ablation procedures, ensuring that energy delivery remains within safe limits to prevent thermal injury to surrounding structures. Furthermore, the system may incorporate impedance monitoring to assess the quality of tissue contact during both pacing and ablation, allowing the control unit to adjust energy delivery in real-time to maintain effective and safe therapeutic outcomes. In some examples, the device may also include a feedback loop that automatically modulates energy output based on sensor data, ensuring consistent and safe ablation.

Another aspect of the systems and methods herein may involve the use of biocompatible and bioactive materials in the construction of the device. The housing of the device may be coated with materials that promote endothelialization, encouraging the growth of a natural endothelial layer over the device's surface. This endothelial layer may help to reduce the risk of thrombus formation and improve biocompatibility, thereby enhancing the long-term safety and efficacy of the device. Additionally, the use of bioactive coatings that release anti-thrombogenic agents may further reduce the risk of clot formation, particularly during the initial period following implantation. In one example, the coating may include a polymer matrix embedded with heparin or other anti-coagulant agents that are gradually released over time.

The device may be constructed with a thrombus-preventive surface incorporating micro-textured, superhydrophobic properties that passively repel platelet adhesion and clot formation. By using a self-cleaning, anti-fouling surface design, the device prevents thrombus buildup without requiring endothelialization-promoting coatings or anti-thrombogenic layers. This micro-textured surface may be created with nano-structured coatings that inhibit platelet attachment, effectively preventing thrombus formation at the LAA opening. This design passively minimizes clot development, allowing for extended implant efficacy without active thrombus management mechanisms.

The system may also be designed to facilitate ease of retrieval and repositioning, if necessary. The device may include features that allow it to be captured and withdrawn through a catheter-based retrieval system, enabling repositioning or removal in the event of device malfunction or changes in patient condition. The retrieval mechanism may include collapsible anchors or a tether that can be engaged by a specialized catheter, allowing for safe and controlled extraction of the device. In one example, the device may incorporate a detachable anchoring system that releases the device in response to an external signal, simplifying the retrieval process and reducing the risk of tissue damage.

Optionally, the systems and methods herein may include a range of advanced features designed to enhance the effectiveness, safety, and adaptability of the system. By incorporating advanced sensing capabilities, dynamic anchoring, drug delivery, multi-modal energy harvesting, wireless communication, safety features, biocompatible materials, and retrievability, the invention provides a comprehensive solution for managing delayed interatrial conduction. These enhancements may contribute to improved patient outcomes by ensuring synchronized atrial contractions, optimizing cardiac function, and reducing the risk of complications associated with traditional pacing systems.

In one example, the system may employ hydrogel-based batteries, which are biocompatible and can be molded into various shapes to fit within the LAA without requiring connectors. These hydrogel-based batteries may utilize a combination of solid electrolytes embedded in a hydrogel matrix, allowing for efficient energy storage and delivery while maintaining the flexibility needed to conform to the complex geometry of the LAA. By eliminating rigid connectors, these batteries can seamlessly integrate into the pacing device's housing, thereby minimizing bulk and maximizing contact with the cardiac tissue. The use of hydrogels also allows the battery to remain securely in place, accommodating cardiac motion without compromising performance.

The device may also utilize nanotechnology to create miniature batteries or energy storage components that can be dispersed within the device rather than relying on a linear chain of connected units. In one example, nanoscale energy storage devices may be distributed throughout the housing of the device, providing a decentralized power source that reduces complexity and potential points of failure. These nanobatteries may incorporate advanced materials, such as carbon nanotubes or graphene, to achieve high energy density while maintaining a compact form factor. By distributing these miniature energy storage units throughout the device, the overall size and rigidity can be reduced, ensuring a more comfortable fit and improving the biocompatibility of the implant.

Another approach may involve the use of solid-state batteries, which can be made thinner and more flexible compared to conventional liquid electrolyte batteries. Solid-state batteries are inherently safer due to the absence of flammable liquid electrolytes, which reduces the risk of leakage or thermal runaway. The use of solid-state batteries in the LAA pacing device may allow the power source to be integrated directly into the housing or other structural components, minimizing the space required for energy storage while enhancing the device's safety profile. These batteries may also be configured to conform to the anatomical features of the LAA, further improving the fit and reducing the potential for mechanical irritation.

In some examples, alternative connector designs may be implemented to address the challenges associated with connecting multiple battery or energy storage units. These connectors may be rigid or fixed, ensuring that each battery subunit remains in a predetermined orientation relative to adjacent subunits. By using rigid connectors, the pacing device may avoid complications related to subunit movement or misalignment, providing a more reliable and mechanically stable power source. This fixed configuration may also simplify the manufacturing process, reducing the likelihood of defects or failures due to misaligned components.

The device may also feature modular snap-fit battery units that can be assembled into a fixed configuration, avoiding the need for flexible connectors altogether. These snap-fit units may be designed to provide a rigid assembly that can be customized based on the specific power needs of the device. For example, additional modules may be added to increase energy capacity, or fewer modules may be used to reduce the size of the implant. By employing a snap-fit mechanism, the battery units can be securely connected without the need for complex assembly tools, simplifying both the manufacturing and implantation processes.

To further enhance the adaptability of the device, the battery units may be arranged in a spiral or helical configuration that conforms to the LAA's unique shape. This spiral configuration may allow the battery components to wrap around the interior of the LAA, providing a more stable and comfortable fit while ensuring even distribution of weight and contact pressure. In one example, the spiral configuration may be designed to expand upon deployment, allowing the device to adapt to the specific dimensions and contours of the LAA without causing undue stress on the cardiac tissue.

Another example may incorporate biodegradable batteries, which are designed to provide power for a limited time before safely dissolving within the body. These biodegradable batteries may be particularly useful for temporary pacing needs, such as during the healing period following a surgical procedure or until a permanent pacing solution can be established. The biodegradable materials used in these batteries may be selected to provide the necessary energy for the duration of the therapy and then degrade naturally, reducing the need for surgical removal and minimizing long-term risks to the patient.

The device may also integrate energy storage directly into its circuitry using advanced materials such as graphene supercapacitors. These supercapacitors may offer rapid charging and discharging capabilities, providing an efficient means of energy storage that eliminates the need for separate battery components. By incorporating energy storage directly into the device's electronic circuits, the overall size and complexity of the implant may be reduced, improving both its reliability and ease of implantation. Graphene supercapacitors, in particular, may provide high energy density and long cycle life, ensuring that the device remains operational for an extended period without the need for frequent recharging or replacement.

To accommodate the diverse morphologies of the left atrial appendage (LAA), the device may be equipped with adaptive design features capable of conforming to varying anatomical structures. The LAA has multiple common morphological types, such as “windsock,” “chicken-wing,” and “cactus” shapes, each presenting unique challenges for device deployment and stabilization. A potential design element to address this variability may be the use of flexible, segmented anchor components. These anchors may be engineered with a multi-segment structure that includes joints allowing the device to articulate, thereby adjusting its shape to adapt to both straight and highly curved LAA morphologies. Each segment may include independently deployable tines that grip the LAA wall, ensuring proper anchoring regardless of the appendage's angle or curvature.

The device may further utilize an expandable scaffold system made of shape-memory alloys, such as Nitinol, which could be pre-programmed to achieve various configurations upon deployment. This scaffold may be heat-set to specific shapes that expand differently based on the morphology encountered. Nitinol is particularly well-suited due to its superelasticity and shape-memory properties, enabling the scaffold to return to a predetermined configuration after being constrained during delivery. The scaffold may take the form of a braided mesh or lattice structure, which can provide enhanced flexibility and conformability to complex LAA geometries. The braided design ensures that the device can expand uniformly and provide consistent coverage, regardless of the variations in LAA anatomy. In some examples, this scaffold may be configured to hold batteries or other power sources for pacing and/or for powering sensors and data processing and communication modules.

In addition to Nitinol, other materials may also be considered for different components of the devices herein. Examples include cobalt-chromium alloys for components requiring additional rigidity and strength, polyethylene terephthalate (PET) or expanded polytetrafluoroethylene (ePTFE) as biocompatible polymers for the covering structure to provide a soft and compliant surface that encourages tissue growth, and titanium or stainless steel for elements where long-term durability and resistance to corrosion are critical.

Bioelastic polymers, such as polyurethane elastomers or silicone elastomers, may be used for components needing flexibility and biocompatibility, ideal for coverings or anchoring segments. Hydrogels may be used where a soft, tissue-like consistency is needed to enhance biocompatibility or promote integration with native tissue. Polyurethane provides high elasticity and durability and is suitable for flexible components that require prolonged exposure to the body's dynamic environment. Silicone elastomers are ideal for parts of the device that must conform to anatomical variations due to their flexibility and stability. Graphene-based coatings may enhance electrical conductivity and ensure biocompatibility of certain electronic components. Phospholipid polymer coatings may provide a biocompatible and anti-thrombogenic surface, reducing platelet adhesion and improving integration with the surrounding tissue. Parylene may be used as a coating material to provide a moisture barrier and enhance the longevity of internal electronics. Carbon composites may be used for lightweight yet strong components that need to provide structural support without adding significant weight. These materials are chosen for their biocompatibility, mechanical properties, and ability to withstand the dynamic environment of the cardiovascular system.

In another example, structural components of the device may be constructed from advanced layered biopolymers or carbon fiber composites instead of or in addition to Nitinol.

These materials offer a comparable level of flexibility and shape memory, enabling the device to adapt to the variable morphologies of the left atrial appendage (LAA). Biopolymers, layered with specific orientations, can achieve resilience and bending properties similar to metal, while carbon fiber composites provide additional tensile strength and durability. Both materials are MRI-compatible and non-metallic, thus avoiding potential artifacts in imaging and enabling a safer profile for long-term implantation. These non-metallic alternatives retain the structural adaptability needed for secure anchoring and precise positioning within cardiac tissue while circumventing the magnetic and thermal constraints associated with traditional Nitinol components.

Another example may include a modular covering structure that changes its surface area to fit varying LAA sizes. This covering may be made of an elastomeric mesh that provides a flexible surface, adjusting its coverage area as needed to form a secure seal at the LAA ostium. This flexibility may be useful for ensuring that the device can both isolate the LAA effectively and minimize the risk of thrombus formation. In addition, the use of helical struts with adjustable tension may facilitate a more precise fit by allowing the device to contour itself along the specific bends and angles of each unique LAA structure. These helical struts may also enable the device to exert uniform pressure, improving stabilization without causing localized stress points that might lead to tissue damage or erosion. In some examples, the cover is a mesh-like structure or scaffold that does not fully occlude the LAA opening and uses electrochemical or biochemical coatings to discourage clot formation.

Optionally, to assist with deployment and conformational adjustment, the devices and systems herein may incorporate a variety of mechanisms. For example, a Nitinol-based expansion device may facilitate deployment by expanding the scaffold into the correct shape upon exiting the catheter. This expansion mechanism would rely on the superelastic properties of Nitinol, allowing it to transition smoothly from a constrained delivery state to its functional form. Inflatable balloon elements may assist in expanding the device into contact with the LAA walls before anchor engagement. Micro-anchor hooks or barbs may provide additional stability by anchoring into the LAA tissue upon deployment. These hooks may be made from biocompatible metals or polymers capable of securely attaching to the tissue without causing undue damage. Alternatively, active fixation mechanisms involving micro-screws or deployable pins may be used to further ensure stability in challenging anatomies.

Another example incorporates micro-motors or actuators within the anchoring system. These micro-motors may allow fine-tuned adjustments after the initial deployment, enabling the device to reposition itself or modify its configuration in real time. Such actuators may be controlled via external commands, providing the physician with enhanced control over the positioning and stabilization process.

The device may also employ an external guidewire system that remains connected during the initial deployment phase. This guidewire may assist in maneuvering the device into the optimal position, particularly in more complex LAA shapes. Once the device is correctly positioned and fully deployed, the guidewire can be detached, allowing the device to remain securely anchored.

Additional deployment and adjustment mechanisms may include polymer-coated springs, which can help in deploying the device by providing a controlled expansion force, and electroactive polymers (EAPs), which can change shape when electrically stimulated, potentially allowing for fine adjustments in the device's configuration after deployment. A hydraulic adjustment mechanism, involving small hydraulic chambers within the device, may be filled or emptied to adjust its shape and ensure optimal conformal contact with the LAA.

Optionally, to address thrombus prevention and promote endothelialization, the device may incorporate a multi-faceted approach that includes both mechanical and biochemical features. The cover portion of the device may be coated with an anti-thrombogenic material, such as heparin or a nitric oxide-releasing polymer, which helps to reduce platelet adhesion and thrombus formation during the critical initial period following implantation. Additionally, the covering may be designed to facilitate endothelialization by incorporating a surface microstructure that mimics the natural extracellular matrix, promoting cell attachment and growth.

Optionally, the devices and systems herein may further include a mesh structure that aligns with the LAA ostium, forming a semi-permeable barrier rather than a complete occlusion. The mesh may be constructed of a biocompatible, anti-thrombogenic material designed to prevent embolic material from exiting the LAA while allowing normal blood flow dynamics. This mesh may deploy as an expandable structure that self-adjusts to the LAA opening without fully capping the LAA, thereby reducing the risk of thrombus formation without obstructing natural atrial flow patterns. By avoiding a complete cap, this design provides a partial physical barrier that does not interfere with atrial blood flow or require complete closure.

To evaluate successful endothelialization in vivo, the device may be equipped with integrated sensors capable of monitoring biomarkers indicative of endothelial health and maturation. These sensors may measure local nitric oxide production, which is a key indicator of healthy endothelial function. Additionally, the device may include electrodes that can measure electrical impedance changes over time, which correlate with the development of an endothelial layer. As endothelial cells proliferate and form a continuous layer over the device, the impedance values will gradually increase, providing a non-invasive method for monitoring endothelialization progress.

Optionally, the devices and systems herein may also utilize imaging modalities to confirm endothelialization. Near-infrared spectroscopy (NIRS) and optical coherence tomography (OCT) might be integrated into the delivery sheath or device itself to provide real-time imaging of the LAA surface. These imaging techniques may allow visualization of the endothelial layer, enabling the physician to verify that endothelial coverage is progressing as expected. The incorporation of fluorescence markers that bind to endothelial cells may also be used in combination with NIRS or OCT to enhance visualization and provide a more detailed assessment of endothelialization.

The device may also integrate imaging and mapping guidance features to ensure precise placement during deployment. Transesophageal echocardiography (TEE) and intracardiac echocardiography (ICE) may be used to provide real-time imaging data that helps guide the device into the optimal position within the LAA. These imaging modalities may be coupled with artificial intelligence (AI) algorithms designed to enhance image interpretation and provide automated suggestions for positioning adjustments. For example, AI-based image analysis may identify key anatomical landmarks, track device movement, and provide real-time feedback to the operator, reducing the risk of improper placement. Additionally, automation features may allow the device to make small, AI-guided adjustments autonomously to enhance positioning accuracy and ensure a secure fit within the LAA.

The anchoring and deployment process may also include feedback mechanisms to enhance safety and efficacy. For example, force sensors integrated into the anchoring elements could measure contact pressure against the LAA walls in real time, ensuring that the deployment force remains within safe limits to prevent tissue damage. These force sensors may be coupled with adaptive control systems that can automatically adjust the deployment force if unsafe levels are detected, thus providing a fail-safe mechanism during the anchoring process.

In an alternative example, the implantable device may be configured to be positioned outside the left atrial appendage while retaining functionality in monitoring and intervention within the atrium. For example, the device may be anchored in the left atrial wall near the LAA using any of the methods described, or flexible, bioadhesive materials that avoid the need for mechanical expansion or radial tines. The anchoring portion may utilize a soft, biocompatible material that adheres securely to the atrial wall upon contact, avoiding invasive penetration or mechanical engagement with the LAA tissue. This design allows the device to capture atrial activity while remaining secure outside the appendage, thereby reducing potential risks associated with anchoring within the LAA and enabling versatile implant positioning. In another example, these materials or methods are employed within the LAA.

To ensure long-term implant viability and extend battery life, the device may be designed with several energy-efficient mechanisms and energy harvesting capabilities. The device may be equipped with a high-capacity lithium-ion battery that has been optimized for low-power consumption, ensuring reliable operation for an extended period. The expected lifespan of the battery may be between 10 to 15 years, depending on the pacing requirements and patient-specific conditions. To further extend battery life, the device may incorporate an energy harvesting system that converts cardiac motion into electrical energy. This system might utilize piezoelectric elements that generate power from the mechanical vibrations produced by the beating heart, effectively reducing the reliance on the internal battery and extending its operational lifespan.

Additionally, the device may be equipped with an advanced power management unit (PMU) that optimizes energy usage by selectively activating different components only when necessary. The PMU may ensure that sensors, actuators, and communication modules operate in a duty-cycled manner, thereby minimizing overall power consumption. The communication module might use low-energy wireless protocols to transmit data, further conserving battery life.

The device may also incorporate a wireless recharging system, allowing an internal power source (such as a battery) to be recharged periodically through an external inductive charging source. This feature might enable patients to extend the lifespan of the implant without requiring invasive procedures for battery replacement. The inductive charging could be done during routine check-ups or at home with a wearable charging vest, providing convenience and reducing the need for surgical interventions.

In terms of maintenance and replacement, the device may be designed to minimize the need for invasive procedures. The battery status and overall device health may be continuously monitored, and data might be transmitted wirelessly to an external monitoring system. This would allow healthcare providers to assess battery levels and schedule replacement procedures proactively before the battery is fully depleted. If replacement is needed, the device may be designed for minimally invasive retrieval using a specialized catheter that can detach the anchoring components and safely extract the device. The new device can then be implanted in the same location, following the same deployment procedure, ensuring continuity of therapy without significant disruption to the patient.

To further enhance long-term viability, the device may include self-diagnostic capabilities. These diagnostics may substantially continuously assess the condition of various components, including sensors, actuators, and battery performance. Any detected anomalies might be communicated to the healthcare provider through the wireless monitoring system, allowing for timely intervention before a critical failure occurs. Additionally, the use of redundancy in critical components, such as multiple sensors or backup actuators, may ensure continued functionality even if one element fails. This redundancy enhances the reliability of the device over its intended lifespan.

The device may also include several safety mechanisms to ensure appropriate energy delivery during anchoring and ablation. One potential safety feature is the incorporation of an energy threshold limiter that prevents the application of excessive energy, which could damage surrounding tissue. The energy delivery system may be equipped with real-time feedback loops that continuously monitor the impedance at the tissue interface. If impedance readings indicate that the tissue is overheating or experiencing excessive resistance, the system might automatically reduce or shut off energy delivery to prevent damage. Additionally, the energy delivery system may include a temperature monitoring feature that measures local tissue temperature at the point of contact. If temperatures exceed a predetermined safe limit, the system may modulate or cease energy application to maintain safe operating conditions.

The anchoring system may use controlled energy pulses to ensure secure fixation while minimizing the risk of thermal injury. The duration, intensity, and frequency of these pulses may be pre-programmed based on the specific LAA morphology and tissue characteristics and may be adjusted in real time based on feedback from sensors integrated into the device. This adaptive energy modulation allows for optimal tissue contact while safeguarding against excessive energy exposure.

For ablation, the device may employ a multi-electrode configuration that allows for distributed energy delivery. This configuration could ensure that energy is applied evenly across the target area, reducing the likelihood of hotspots that could lead to tissue necrosis. The system may also use a closed-loop control algorithm that monitors tissue response during ablation, adjusting energy output as needed to achieve effective ablation without causing collateral damage. These safety parameters and feedback mechanisms could work in conjunction to provide a controlled and effective energy delivery process, enhancing both the safety and efficacy of the device.

The device may also feature adaptive control systems to interpret real-time data from sensors for pressure, impedance, and force detection, allowing it to adjust its behavior dynamically. The control algorithms may utilize a combination of proportional-integral-derivative (PID) control loops and machine learning models to optimize the device's response to changing physiological conditions. The PID control loops may provide precise and immediate adjustments based on sensor input, ensuring that parameters such as pressure and force are kept within safe limits. For more complex adaptations, such as responding to unexpected variations in LAA morphology or tissue elasticity, the device may employ machine learning algorithms that analyze historical and real-time data to predict optimal adjustments.

The control system may integrate a decision-making framework that prioritizes patient safety by continuously assessing sensor data against predefined thresholds. If pressure sensors detect an increase beyond safe levels, the system could immediately adjust the deployment force or reduce energy delivery to prevent tissue injury. Similarly, if force detection sensors indicate a potential risk of perforation, the control system could trigger a retraction or repositioning of the device to mitigate the risk.

The impedance sensors may be used in conjunction with adaptive algorithms that monitor tissue contact quality during both anchoring and ablation. If impedance values deviate from expected norms, indicating poor contact or overheating, the system might recalibrate energy application to optimize efficiency and safety. The machine learning component of the control system could enable the development of individualized patient profiles, enabling the device to learn from each deployment and make more informed adjustments in future procedures.

The device may also include communication capabilities to interact with other implanted devices, such as ventricular pacemakers, using secure and power-efficient protocols. The communication module might utilize low-energy Bluetooth (BLE) and radio frequency (RF) technologies to establish a reliable connection with other medical implants. To mitigate potential interference, the device may employ frequency hopping spread spectrum (FHSS) techniques, which change the communication frequency at regular intervals, reducing the likelihood of signal disruption and improving communication stability.

In some examples, the device may be equipped with an acoustic transmitter to enable intra-cardiac synchronization with other implanted devices. This transmitter may utilize low-frequency acoustic signals to communicate operational status or synchronization signals without reliance on traditional wireless frequencies, thus reducing energy consumption. By using acoustic waves, the device can achieve short-range communication with other atrial or ventricular implants while conserving battery life. The acoustic-based system may also allow for periodic data synchronization with external monitoring systems via an intermediary device, facilitating real-time monitoring of atrial functions without the need for Bluetooth or low-energy radio frequency communication.

In another example, to facilitate synchronization with other cardiac devices, the implantable device may use passive vibration-based communication. This method involves short-range mechanical signals generated by minor oscillations or tapping within the device housing, detected by adjacent implants within the body. Unlike traditional Bluetooth or acoustic-based protocols, this approach requires minimal power and enables device synchronization through mechanical resonance. The vibrations may be calibrated to encode specific signals, facilitating communication with ventricular or atrial pacemakers without relying on radio frequencies or sound waves, thereby optimizing energy efficiency.

Optionally, to further enhance the security of communication, all data transmitted between devices may be encrypted using advanced encryption standards (AES) to protect patient information and prevent unauthorized access. The communication module may operate on a duty-cycled basis, wherein the device only communicates at predefined intervals or when triggered by an external event, thereby conserving power. Additionally, the system might include error-checking algorithms to ensure data integrity during transmission and employ acknowledgment protocols to confirm successful data exchange.

The communication protocols may be designed to be interoperable with various types of cardiac implants from different manufacturers, ensuring that the device can seamlessly integrate into existing implant ecosystems. The communication module might adhere to established medical communication standards, such as IEEE 11073, to promote compatibility and interoperability. This ensures that the device can share critical data, such as pacing status or hemodynamic parameters, with other implanted devices, allowing for coordinated therapeutic interventions and enhanced patient monitoring.

For post-implant monitoring, the device may include remote monitoring features to allow healthcare providers to assess device performance and patient status on an ongoing basis. The device may transmit data to a secure, cloud-based platform that healthcare providers can access to track metrics such as battery status, pacing efficiency, and hemodynamic changes. This remote monitoring system might also include alert mechanisms that notify healthcare providers in case of anomalies, such as a drop in battery levels or unusual hemodynamic patterns, enabling timely intervention.

Optionally, patients may also be provided with a handheld or wearable interface that allows them to interact with the monitoring system. This interface might display basic information about device function, such as battery levels, and alert patients to contact their healthcare provider if necessary. Routine follow-up appointments may also be complemented by data collected through remote monitoring, reducing the need for frequent in-person visits while ensuring continuous and comprehensive patient care.

Overall, incorporating one or more of segmented anchors, shape-memory alloy scaffolds, modular coverings, pneumatic balloon systems, micro-motors, an external guidewire system, an endothelialization monitoring system, imaging and mapping guidance, energy-efficient mechanisms, wireless recharging, self-diagnostic capabilities, safety mechanisms for controlled energy delivery, adaptive control systems, secure communication protocols, and post-implant remote monitoring may significantly enhance the ability of any of the devices and systems herein to conform to different LAA morphologies, prevent thrombus formation, ensure proper endothelial growth, maintain long-term viability, and communicate effectively with other implants. These elements could work in concert to provide a tailored fit for each patient, improving the efficacy and safety of LAA isolation during atrial fibrillation treatment.

An example of the device may include a single battery-powered leadless pacemaker, designed for placement within the left atrial appendage (LAA) via a catheter delivery system. The device is a miniaturized, self-contained unit encapsulated within a biocompatible housing suitable for the unique environment of the LAA. It may comprise an internal battery power source, a pulse generator, sensing circuitry, and electrodes that interface directly with cardiac tissue. Upon deployment, the pacemaker may anchor itself to the LAA wall using fixation mechanisms such as tines or helixes, allowing it to provide pacing and sensing functions without the need for traditional transvenous leads. This design may ensure a minimally invasive solution for treating patients with cardiac conduction abnormalities or arrhythmias.

To sustain long-term functionality, the device may incorporate multiple energy harvesting mechanisms that convert natural physiological movements and other forms of available energy into electrical power. One such system may be a mechanical energy harvesting module that utilizes cardiac motion. This module may be composed of piezoelectric materials strategically placed within the LAA to undergo deformation in synchrony with the heart's contractions and relaxations. As the heart beats, these piezoelectric elements may be deformed, inducing electrical charges that can be harnessed to power the pacemaker's onboard electronics, including pacing and sensing functionalities.

In some examples, the device may integrate a thermoelectric generator that utilizes the temperature difference between the body's core temperature and the cooler blood in the LAA. This thermoelectric module may contain thermocouples arranged to maximize the Seebeck effect, generating electrical power from the natural physiological temperature gradients. Such an energy source may provide a continuous supply of electricity to maintain device function.

In addition to mechanical energy harvesting, the device may integrate nanogenerators composed of piezoelectric nanowires or thin films to capture subtle vibrations of cardiac tissue and blood flow within the LAA. These nanogenerators may generate electricity continuously, which helps sustain device operations without the reliance on an internal battery. This energy harvesting strategy may be particularly advantageous for ensuring a consistent power supply, especially in a challenging implant environment like the LAA.

The device may also feature a blood flow turbine-based energy harvesting unit, consisting of micro-scale turbines or flexible flaps positioned within the LAA to capture the kinetic energy from pulsatile blood flow. The movement of these turbines may drive a small generator, converting the kinetic energy into electrical power, which may then be stored and regulated to meet the functional requirements of the pacemaker. Such a system may capitalize on the natural hemodynamics of the heart to generate power continuously, minimizing the need for frequent recharging or battery replacements.

Another energy harvesting technique that may be utilized in this device may be based on electromagnetic induction. The device may include conductive coils and magnets that create a magnetic field, and as the conductive blood moves through this field, electrical currents are induced. This electromagnetic induction may provide a sustainable power source for the device's operations, allowing it to function autonomously for extended periods.

In some examples, the device may incorporate triboelectric nanogenerators that harvest energy from frictional interactions between device surfaces and moving cardiac tissues or flowing blood. This triboelectric effect may generate electrical charges that are then converted into power to support device functionalities.

To further extend the device's operational life, the system may be equipped with a wireless power transfer capability through inductive coupling. A receiver coil may be embedded within the device, capable of receiving electromagnetic energy transmitted from an external power source. Patients may wear an external transmitter over the implantation site, which generates an alternating magnetic field to wirelessly power the device or recharge its battery. This method of power transfer may provide a convenient, non-invasive means for maintaining adequate energy levels within the implanted device.

For additional flexibility, the device may incorporate a radiofrequency (RF) energy transfer module, where an external RF transmitter may deliver energy to an implanted antenna and resonant circuitry. This RF energy may then be converted into electrical power, ensuring that the device can continue to function effectively. The external transmitter may be programmed to operate at specific frequencies and intervals to optimize power transfer efficiency and minimize the patient's inconvenience.

In some examples, an ultrasound-mediated power transfer system may be included in the device. It may comprise a piezoelectric element capable of converting acoustic energy into electrical power. An external ultrasound transducer, when activated, may emit focused ultrasound waves towards the implanted device, and the piezoelectric component may then convert these acoustic signals into electricity. This method may allow transcutaneous energy transfer without the need for direct electrical coupling, adding another layer of convenience for recharging or powering the device.

The device may also incorporate advanced power generation methods, such as optical energy transfer using photovoltaic cells. These cells may be sensitive to near-infrared (NIR) light and convert externally applied optical energy into electrical power. An external NIR light source may be used to project light through the patient's tissue towards the implanted device, which may then absorb and convert this light into electricity. Mechanisms for alignment or focusing the incoming light onto the photovoltaic cells may be integrated into the device, ensuring optimal energy absorption and power conversion.

Additionally, the device may include a biofuel cell capable of generating electrical energy through enzymatic reactions involving glucose and oxygen naturally present in the bloodstream. Bio-electrodes coated with specific enzymes may catalyze the oxidation of glucose at the anode and the reduction of oxygen at the cathode, producing a flow of electrons that powers the device's electronic components. This biofuel cell approach may provide a continuous source of power, leveraging the body's natural metabolic processes to sustain the device's operations.

In some examples, the device may include supercapacitors for rapid energy storage and discharge. Supercapacitors may provide high power density and long cycle life, enabling them to store energy captured by various harvesting mechanisms and release it as needed for pacing or sensing functions.

In some examples, the electronics may integrate with existing LAA closure devices. The closure component may serve as a scaffold for embedding sensors, electrodes, and power generation modules, enabling the multifunctional device to simultaneously provide pacing or sensing functions and effectively isolate the LAA to prevent thromboembolism.

In some examples, the device may incorporate shape-memory alloys (SMAs) that change shape in response to temperature changes or electrical stimuli. These SMAs may facilitate deployment and secure anchoring within the LAA by expanding or contracting upon activation, ensuring a stable and conforming fit within the cardiac anatomy, and reducing the risk of device migration.

The anchoring system may utilize a shape-memory polymer that expands upon reaching body temperature, providing a flexible, adaptive fit within the LAA. This polymer may be embedded with micro-adjustable hinges that allow for dynamic shape conformance without the need for Nitinol or metallic components. The device may incorporate a radial expansion triggered by exposure to physiological temperatures, securing the device within the LAA without using tines, screws, or radial force. This polymer-based anchoring system may be further enhanced by a self-expanding lattice structure that can collapse for device insertion and return to its original shape for secure anchoring, enabling gentle, tissue-conforming engagement with the atrial appendage.

The device may also utilize advanced materials such as graphene or carbon nanotubes to enhance its performance. These materials, known for their exceptional electrical conductivity and mechanical strength, may be used in constructing electrodes, energy storage components, or energy harvesting elements, contributing to miniaturization, durability, and improved device functionality.

The pacemaker system may include a rechargeable battery module that can be replenished wirelessly through external means. Inductive or RF-based recharging systems may allow the patient to restore the battery's charge periodically without invasive procedures. Built-in safeguards may prevent overcharging and ensure patient safety during recharging, making the system practical for long-term use. By incorporating a combination of these advanced energy harvesting and power management techniques, the device may maintain consistent functionality, reducing the frequency of surgical interventions and enhancing patient quality of life.

In some examples, the device may also be equipped with thermoelectric generators, triboelectric elements, or magnetohydrodynamic (MHD) systems that generate electrical power from natural body movements, temperature gradients, or blood flow interactions with magnetic fields. The device may further include artificial organelle-based energy production modules or integrate biological circuits to perform specific energy-generating functions. Such versatility may ensure that multiple energy harvesting mechanisms work in tandem to reliably sustain the device's operations.

In some examples, the device may include advanced artificial intelligence (AI) algorithms that autonomously analyze sensor data and make decisions to optimize therapeutic delivery. The AI may adapt to the patient's physiological conditions in real time, adjusting pacing rates, detecting arrhythmias, or modifying energy harvesting strategies for maximum efficiency.

In some examples, the device may incorporate micro-electromechanical systems (MEMS), featuring microscale components like sensors, actuators, and valves, allowing precise manipulation of device functions for localized therapeutic interventions.

In some examples, the device may also employ thermal energy harvesting mechanisms that utilize minor temperature fluctuations between body heat and surrounding blood to generate electrical power, providing additional energy without relying solely on mechanical movement.

The device may also be equipped with integrated biochemical sensors capable of detecting specific physiological markers, such as pH levels, oxygen saturation, electrolytes, or proteins. These sensors may provide diagnostic information, enabling the device to adjust therapeutic functions in response to real-time physiological changes.

To accommodate short-term or targeted applications, in some examples, the device may be constructed from biodegradable or bioresorbable materials, allowing it to dissolve within the body after fulfilling its therapeutic purpose. This may eliminate the need for retrieval and reduce the risks associated with long-term implantation. The device may also be equipped with bioadhesive surfaces for enhanced attachment within the LAA or may incorporate microneedles or microelectrodes to penetrate the endocardial tissue for high-fidelity sensing and stimulation.

In some examples, the device may incorporate optogenetic functionality to modulate genetically modified cells using specific wavelengths of light. This optogenetic approach may be used for precise control of cellular activity, potentially improving cardiac function and reducing arrhythmias.

In some examples, the device may employ a swarm technology system, where multiple miniaturized devices are implanted collaboratively within the LAA. These devices may wirelessly communicate to perform distributed sensing, coordinated pacing, or collective energy harvesting, enhancing the overall system's robustness and therapeutic efficacy.

In some examples, the device may leverage bio-inspired design principles, mimicking natural biological structures such as the helical patterns of cardiac muscle fibers. This bio-inspired approach may improve biocompatibility and integration with cardiac tissue, resulting in more effective therapy delivery.

In some examples, the device may utilize a range of innovative battery technologies to support long-term power needs. Miniaturized primary batteries, such as lithium-based cells, may be incorporated to provide a compact and high-energy-density power source. Thin-film solid-state batteries may also be used, offering a flexible, safe, and reliable power supply. These thin-film batteries may conform to the device's shape, improving compatibility with the LAA and maximizing space efficiency.

The device may also use rechargeable batteries, such as lithium-ion or lithium-polymer cells, with wireless recharging capabilities. In some examples, microbatteries may be integrated directly into the device structure, including arrays of micro-scale batteries embedded in non-functional areas to maximize energy storage without increasing overall device size. Hybrid battery systems combining batteries and supercapacitors may be used to leverage the high energy density of batteries and the rapid charge-discharge capabilities of supercapacitors, extending battery life and improving device performance.

In some examples, biocompatible flexible batteries made from flexible polymers and biocompatible electrodes may be used, allowing the battery to bend and flex with cardiac movements, reducing mechanical stress on the device and surrounding tissues. Biofuel-powered batteries that generate power through biochemical reactions using substrates like glucose and oxygen from the bloodstream may also be incorporated to provide a continuous power source.

In some examples, advanced battery chemistries, such as lithium-air, lithium-sulfur, or solid-state electrolytes, may be employed to increase energy density and safety. Encapsulated battery modules may be used, where the battery is enclosed within biocompatible materials to prevent interaction with bodily fluids. Battery arrays configured for redundancy may also be employed to ensure reliable power supply, with multiple small batteries providing power even if one fails.

In some examples, microfabricated batteries using MEMS technology may be integrated into the device, allowing for compact and efficient designs. Battery integration with device anchoring mechanisms may also be employed to optimize space utilization and improve weight distribution, aiding in stable anchoring and reducing the risk of migration.

To further enhance safety, battery modules with integrated safety features, such as overcharge protection, temperature monitoring, and pressure relief mechanisms, may be used. These features may prevent battery failure modes that could compromise device integrity or patient safety.

In some examples, biodegradable batteries made from materials that safely dissolve in the body over time may be employed for temporary devices. These batteries may provide power for the required duration and then degrade, eliminating the need for battery retrieval and reducing long-term risks associated with residual materials.

In some examples, the device may utilize disposable battery capsules that can be replenished via minimally invasive procedures. The capsules may contain the battery and can be detached and replaced when depleted, allowing for battery replacement without removing the entire device.

In some examples, the device may incorporate advanced power management circuitry that optimizes battery usage by controlling power supplied to different device functions based on necessity. Features like sleep modes, duty cycling, and adaptive pacing algorithms may reduce power consumption and extend battery life. The device may also include rechargeable batteries that can be charged on-demand using an external energy source, with the patient or clinician initiating charging sessions as needed.

Overall, by integrating a wide range of energy harvesting techniques, innovative battery technologies, advanced materials, secure anchoring mechanisms, innovative power management strategies, artificial intelligence, and bio-inspired designs, the device may autonomously provide therapeutic and diagnostic functionalities within the LAA for an extended period. These features may collectively enhance its applicability for treating a variety of cardiac conditions, offering a versatile and minimally invasive solution for patients.

The device may physically occlude the opening of the LAA while incorporating electronic components for pacing, sensing, or therapeutic functions. It may consist of a self-expanding metal frame made from materials like nitinol or other shape-memory alloys, covered with a permeable or impermeable membrane. Upon deployment, the frame may expand to engage the walls of the LAA securely. Electronic components such as sensors, electrodes, and power sources may be integrated into the frame or membrane. The membrane may serve both to isolate the LAA and as a platform for electronic integration.

The device may include a collapsible framework that expands upon deployment to conform snugly to the LAA's internal anatomy, providing a stable platform for electronic components. Constructed from flexible materials, the device may be constrained during catheter delivery and expand when released into the LAA. It may feature anchoring elements like radial force mechanisms or flexible struts that press against the LAA walls, ensuring consistent contact and secure anchoring, accommodating various LAA sizes and shapes. The device may also feature a helical or corkscrew shape that anchors by being gently screwed into the LAA tissue. Delivered via a rotational catheter system, the device may be rotated during deployment to embed its helical structure into the LAA wall. The central shaft may house electronic components, and the helical threads may act as both the anchoring mechanism and the electrical interface with cardiac tissue.

The device may open like an umbrella or disc within the LAA to provide occlusion and anchoring. Delivered in a collapsed state, the device's canopy may expand upon deployment to cover the LAA orifice. Peripheral anchors or hooks may secure it to the LAA walls. The umbrella surface may be embedded with sensors, electrodes, or energy-harvesting components, ensuring even distribution of force and minimizing pressure points on the tissue. A plug-shaped device may feature expandable lobes or flanges that anchor into the LAA. Inserted into the LAA opening, the device's lobes may expand outward to press against the walls, securing it in place. The central body may contain the electronics and power source. The plug may serve the dual purpose of anchoring the device and occluding the LAA to prevent blood flow into the appendage.

The device may also be a mesh structure that expands to fill the LAA, providing anchoring and a surface area for electronic integration. Made from self-expanding materials, the device may be compressed during delivery and expand within the LAA. The mesh may conform to the appendage's internal contours, and its struts may incorporate electrodes and sensors. It may exert gentle radial force to remain in place without damaging the tissue. The device may feature biomimetic designs that mimic biological anchoring mechanisms, such as tendrils or barbed structures, to enhance integration with cardiac tissue. Featuring micro-scale barbs, hooks, or Velcro-like surfaces, the device may engage with the natural trabeculations of the LAA. These structures may provide secure anchoring while minimizing trauma and promoting endothelialization, potentially reducing the risk of thrombosis.

A device may use biocompatible adhesives to bond with the LAA tissue. Upon deployment, the device may apply a medical-grade adhesive to its contact surfaces, creating a strong bond with the endocardial tissue. This method may avoid mechanical fixation, reducing the risk of tissue perforation or damage. The device may also anchor by inflating a balloon within the LAA to fill the space and press against the walls. It may include a deflated balloon during delivery, which may be inflated with a biocompatible fluid or gas upon placement. The balloon may conform to the LAA shape, providing stable anchoring. Its surface may be embedded with electrodes or sensors and may include a self-sealing mechanism to maintain inflation.

The device may use mechanical anchors in conjunction with membranes to both secure and isolate the LAA. It may deploy mechanical anchors such as hooks or expandable frames and include a membrane or mesh that covers the LAA entrance. The membrane may provide a barrier to blood flow, aiding in thrombosis prevention, while the anchors ensure device stability. The device may also anchor by attaching sutures to the LAA tissue. Specialized delivery tools may place sutures through the device and into the LAA wall. Once secured, the sutures may be tied or fixed with clips, anchoring the device. This method may allow for precise positioning and adjustment during the procedure.

The device may use magnetic attraction to anchor within the LAA. It may contain magnetic elements that interact with magnetized particles delivered to or coated on the LAA tissue. Magnetic forces may hold the device in place without mechanical penetration, potentially reducing tissue trauma. The device may be equipped with claw-like appendages that grip the LAA tissue upon deployment. The claws may be folded during delivery and extend outward when the device reaches the LAA. They may gently grasp the tissue, providing secure anchoring with atraumatic tips to minimize injury.

The device may utilize hydrogels that expand upon absorbing bodily fluids to anchor within the LAA. Components made of hydrogel materials may swell after exposure to blood, filling the LAA cavity and securing the device through mechanical pressure. The hydrogels may be biocompatible and designed to degrade over time if temporary anchoring is desired. The device may also be a mesh structure with integrated barbs that anchor into the LAA tissue. Deployed within the LAA, the mesh's barbs may engage with the trabeculated inner surface, providing a scaffold for electronic components. The mesh may be customized to match the patient's anatomy for optimal fit.

The device may include flexible tails or filaments that extend into the LAA for anchoring. The tails may become entangled within the natural trabeculations of the LAA, anchoring the device through friction and mechanical interaction without penetrating the tissue. The device may adhere to the LAA walls using suction created by negative pressure. It may include suction cups or chambers that, when activated, create a vacuum effect, causing it to adhere to the tissue. This anchoring method may avoid mechanical fixation and can be reversed if repositioning is necessary.

The device may deploy a biocompatible foam within the LAA to provide anchoring and occlusion. Upon deployment, the device may release a foam that expands to fill the LAA. The foam may solidify, anchoring the device and isolating the LAA. It may be formulated to be biodegradable if temporary occlusion is desired. The device may be composed of multiple parts that assemble and lock together within the LAA. Delivered sequentially via catheter, the components may interconnect within the LAA, forming a cohesive structure. This approach may allow larger devices to be implanted through smaller catheters, providing secure anchoring through mechanical interlocking.

The device may utilize materials that change properties in response to stimuli like temperature or pH. Made from shape-memory polymers or alloys, the device may remain flexible during delivery and become rigid upon exposure to body temperature or specific chemical environments. This change may enhance anchoring and structural integrity. The device may partition the LAA into smaller sections to prevent thrombus formation and provide anchoring surfaces. It may extend frameworks or barriers within the LAA, reducing chamber size and limiting blood stasis. These structures may anchor the device and promote tissue integration.

The device may anchor by exerting outward radial force against the LAA walls. Similar to vascular stents, the device may expand to press uniformly against the LAA interior, providing frictional anchoring without penetrating the tissue. It may accommodate different LAA sizes. The device may be designed with surfaces that encourage tissue growth into them, enhancing long-term anchoring. Featuring porous surfaces or bioactive coatings, the device may promote endothelial cell migration and tissue ingrowth. Over time, it may become integrated with the cardiac tissue, providing stable anchoring and reducing dislodgement risk.

The device may include anchoring elements that may be adjusted post-deployment to optimize positioning. For example, the device may include components that can be expanded, contracted, or repositioned using external controls or specialized catheters, allowing fine-tuning of the device's position and anchoring force after deployment. The device may also feature flexible joints that accommodate cardiac motion, reducing stress on anchoring points. Incorporating ball-and-socket or flexible joint mechanisms, the device may move with the heart's contractions, minimizing mechanical stress on both the tissue and the device, potentially increasing durability and anchoring reliability.

These mechanical configurations provide a range of strategies for designing implantable devices that anchor securely within the LAA and effectively isolate it from the left atrium. By carefully considering factors such as anatomical variability, deliverability, stability, and integration with electronic components, these designs aim to enhance patient outcomes while minimizing risks associated with implantation. Key considerations for mechanical implementation include ensuring biocompatibility, deliverability, stability, retrievability, promoting endothelialization, minimizing thrombus formation, and supporting the incorporation of electronic components without compromising mechanical function. By combining these mechanical designs with advanced electronic capabilities, implantable devices in the LAA may provide effective therapies for cardiac conditions while ensuring patient safety and device longevity.

In one example, the systems and methods herein may provide a system for estimating LA pressure and predicting heart failure exacerbation by analyzing mechanical and electrical signals from the LAA. The system may include an implantable device positioned within the LAA, containing sensors to detect mechanical activity of the LAA. A data acquisition module collects and synchronizes mechanical sensor data and ECG signals, and a processing unit analyzes the data, estimates LA pressure, and predicts clinical outcomes such as heart failure hospitalization.

The device be may designed for implantation within the left atrial appendage. It may be constructed from biocompatible materials suitable for long-term implantation. In one example, the device features a cap or cover designed to occlude the LAA. This component acts as a structure on which sensors are mounted, providing stability and ensuring close contact with the atrial wall.

Optionally, the device may include various types of sensors for detecting mechanical events within or outside of the LAA. Strain gauges may be affixed to the cap or cover to detect mechanical deformation of the LAA. These sensors are highly sensitive to changes in the stretching or compression of the LAA, allowing for an accurate estimation of LA pressure. Accelerometers may detect vibrations and movements associated with the cardiac cycle, including the a, c, and v waves, providing data on the timing and amplitude of mechanical events within the LAA. In some implementations, pressure sensors may be used to directly measure changes in LA pressure, providing high-accuracy data to further refine the LA pressure estimation. Bioimpedance sensors may measure changes in the electrical impedance of the atrial tissue, which varies based on blood volume and tissue compliance, complementing other mechanical measurements. Acoustic sensors, such as microphones, may also be included to detect the sounds produced by cardiac activity, including heart valve closures and flow-related turbulence. These sounds can be correlated with mechanical events to provide additional insights.

The device may include a non-mechanical pressure sensing system that estimates left atrial pressure by indirect means, such as acoustic or optical measurements. An embedded optical sensor may capture subtle refractive index changes within or outside of the LAA, correlating to left atrial pressure fluctuations. In another example, an ultrasonic transducer can emit low-power pulses to detect atrial wall motion and infer pressure changes through waveform analysis. This approach avoids the use of piezoelectric elements or strain gauges by implementing non-invasive techniques to assess hemodynamic parameters, thereby providing a continuous LAP estimation with minimal contact.

The system may include an ECG module that provides electrical signals corresponding to the cardiac cycle, including P-waves, QRS complexes, and T-waves. These electrical signals serve as a reference for correlating the timing of mechanical events detected by the sensors within the LAA. The data acquisition module is responsible for collecting signals from the mechanical sensors and ECG module. The system ensures that all collected data is accurately time-stamped to synchronize the electrical and mechanical events. The data may then be transmitted wirelessly to an external processing unit or clinician's workstation for further analysis.

The processing unit may be configured to analyze the synchronized electrical and mechanical data to estimate LA pressure and predict heart failure hospitalization risk. The processing unit may measure time delays between electrical events, such as the P-wave, QRS complex, and T-wave detected by the ECG module, and corresponding mechanical responses, such as the a, c, and v waves detected by the sensors. The processing unit may analyze the characteristics of the mechanical signals, including amplitude, frequency, and phase, as well as the time delays between electrical and mechanical events. Changes in these parameters are indicative of changes in LA pressure and compliance of the atrial wall. Machine learning algorithms may also be employed to model the relationship between sensor data and LA pressure. The system may be trained using datasets with known LA pressure values obtained through invasive methods, allowing for the prediction of LA pressure in real time. The processing unit may use the estimated LA pressure and other physiological markers to predict heart failure exacerbation or hospitalization risk. Patterns or trends in the data may be analyzed to identify early signs of worsening cardiac function.

The cardiac cycle is characterized by a series of electrical and mechanical events. The ECG provides electrical signals that correspond to atrial and ventricular depolarization and repolarization. These electrical events initiate mechanical responses in the heart, including atrial contraction (a wave), ventricular contraction impacting the atrium (c wave), and venous filling (v wave). The P-wave represents atrial depolarization, which is followed by atrial contraction (a wave). The system may measure the time delay between these events to estimate atrial compliance and pressure. The QRS complex represents ventricular depolarization, which causes the mitral valve to bulge into the left atrium, creating the c wave. The time delay between these events may be analyzed to assess left atrial pressure. The T-wave represents ventricular repolarization, and the v wave corresponds to passive filling of the left atrium. The timing and magnitude of this mechanical event are indicative of the pressure within the left atrium.

Changes in LA pressure affect the compliance of the atrial wall, which in turn affects the timing and magnitude of the mechanical events. By measuring the time delays between electrical signals from the ECG and the corresponding mechanical responses, the system may estimate LA pressure. Increased LA pressure typically results in delayed or dampened mechanical responses due to reduced compliance of the atrial wall.

The system may provide real-time, continuous estimation of LA pressure, allowing clinicians to monitor patients with heart failure remotely. By detecting early signs of elevated LA pressure, the system facilitates timely interventions, potentially preventing hospitalizations and improving patient outcomes. For patients with atrial fibrillation, the system may detect changes in LA pressure that indicate an increased risk of arrhythmic events. Continuous monitoring of LA pressure can guide treatment adjustments to maintain optimal cardiac function. In patients with delayed interatrial conduction, the system may be used to optimize atrial pacing. By analyzing the timing of mechanical events in the left atrium relative to the right atrium, pacing parameters may be adjusted to improve atrial synchrony and overall cardiac efficiency.

Optionally, the systems herein may incorporate additional sensors to enhance the accuracy of LA pressure estimation and outcome prediction. These sensors may include optical sensors, such as photoplethysmography (PPG) sensors, which detect changes in blood volume within the atrium, providing additional data on cardiac function. Ultrasound sensors may also be used to detect detailed mechanical activity within the heart, including wall motion and valve function. The system may employ adaptive machine learning models that personalize LA pressure estimation based on individual patient data. These models can learn from each patient's unique physiology, improving the accuracy of predictions over time.

While the devices herein are generally described as being implanted within the LAA, the devices may be adapted for placement in other cardiac regions where LA pressure estimation is feasible. For example, the device may be positioned in the left atrium itself or in other adjacent structures to capture relevant mechanical activity. The device may include power management features to extend battery life, possibly incorporating energy harvesting technologies. Wireless communication may be encrypted to ensure data security and patient privacy, complying with regulatory requirements.

The systems and methods herein may be applicable in the development of implantable cardiac monitoring systems. For example, the system may provide an approach to non-invasively estimate LA pressure, benefiting patients with cardiovascular diseases by enabling continuous monitoring and improved management of their conditions. The system may also be integrated into comprehensive heart failure management programs to optimize patient care and reduce healthcare costs.

In some examples, the system may be integrated with wearable devices to provide additional physiological data, such as activity level or respiratory rate. This integration could further improve the accuracy of heart failure exacerbation predictions by considering factors beyond LA pressure. The system may also be expanded to include a closed-loop therapy component, wherein LA pressure estimates are used to automatically adjust therapeutic interventions, such as atrial pacing or medication delivery. This approach may enable real-time, adaptive treatment of heart failure and other cardiac conditions.

For example, the systems may include advanced data analysis techniques, such as deep learning algorithms, to enhance the system's ability to detect subtle patterns in the data. These algorithms may improve the early detection of heart failure exacerbations and provide personalized treatment recommendations. The system may be expanded to include a multi-sensor array within the LAA or other cardiac regions, providing a more comprehensive view of cardiac mechanics. The use of multiple sensors could improve the accuracy and robustness of LA pressure estimation and clinical outcome predictions.

It is fully contemplated that the features, components, and/or steps described with respect to one or more examples, methods, or figures herein may be combined with the features, components, and/or steps described with respect to other examples, methods, or figures of the present disclosure.

Further, in describing representative examples, the specification may have presented the method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

We claim:

1. A system for treating a patient, comprising:
a cardiac device sized for implantation within or near the left atrial appendage of a heart;
a cap configured to cover at least a portion of the device when positioned within the left atrial appendage;
a power source configured to supply energy to the device;
a communication component configured to transmit data from the device to a receiver;
one or more sensors integrated with the cap and configured to detect mechanical changes in the cap associated with variations in left atrial pressure; and
a processor configured to receive data from the one or more sensors and estimate left atrial pressure or changes in left atrial pressure in the heart based at least in part on the mechanical changes detected by the one or more sensors.

2. The cardiac device of claim 1, wherein the one or more sensors comprise one or more strain gauges.

3. The cardiac device of claim 1, wherein the one or more sensors comprise one or more piezoelectric elements.

4. The cardiac device of claim 1, further comprising an accelerometer configured to detect motion or vibrations associated with cardiac activity.

5. The cardiac device of claim 1, further comprising a microphone configured to capture heart sounds for diagnostic purposes.

6. The cardiac device of claim 1, further comprising a pressure sensor carried by the device, wherein the processor is configured to periodically calibrate the estimated left atrial pressure by cross-referencing the mechanical changes detected by the one or more sensors with real pressure data from the pressure sensor.

7. The cardiac device of claim 1, wherein the processor is further configured to store historical data of left atrial pressure measurements in memory.

8. The cardiac device of claim 1, wherein the processor is configured to analyze trends in left atrial pressure over time and adjust operation of the device base at least in part on the trends.

9. The cardiac device of claim 1, wherein the processor is configured to analyze the estimated left atrial pressure data to detect early signs of worsening heart failure.

10. The cardiac device of claim 1, wherein the processor is configured to trigger an alert when the estimated left atrial pressure exceeds a predetermined threshold.

11. A system for treating a patient, comprising a cardiac device for configured for placement within a left atrial appendage of a heart;
one or more mechanical sensors carried by the device and configured to detect mechanical changes corresponding to left atrial pressure;
at least one electrical sensor configured to measure electrocardiogram (ECG) signals of the patient;
a power supply; and
a processor configured to:
receive signals from the one or more mechanical sensors and the at least one electrical sensor; and
estimate changes in left atrial pressure in the heart based at least in part on comparing time delays between the ECG signals and the mechanical signals.

12. The cardiac device of claim 11, wherein the one or more mechanical sensors comprise one or more accelerometers configured to detect mechanical vibrations corresponding to the estimated left atrial pressure.

13. The cardiac device of claim 11, wherein the one or more mechanical sensors comprise one or more strain gauges configured to detect strains corresponding to the estimated left atrial pressure.

14 The cardiac device of claim 11, wherein the processor is further configured to identify components of atrial pressure waveform, including at least one of the following: an a wave, c wave, and v wave of the heart.

15. The cardiac device of claim 14, wherein the processor is configured to estimate the left atrial pressure based at least in part on analyzing changes in timing and amplitude of mechanical signals.

16. The cardiac device of claim 15, wherein the processor is configured to determine a patient's volume status based on the changes in the timing and the amplitude of the mechanical signals.

17. The cardiac device of claim 11, further comprising a leadless pacemaker configured to deliver pacing pulses.

18. The cardiac device of claim 17, wherein the pacemaker is programmed to adjust a heart rate of the heart to enhance identification of the patient's volume status.

19. The cardiac device of claim 11, wherein the processor is configured to align mechanical measurements with the ECG signals to correlate mechanical events with specific phases of a cardiac cycle of the heart.

20. The cardiac device of claim 11, wherein the processor is configured to perform waveform analysis to identify and analyze components of a atrial pressure waveform.

21. A method for estimating left atrial pressure in a patient using a cardiac device implanted within or near a left atrial appendage of a heart of the patient, the method comprising:
detecting mechanical changes in a cap of the device associated with variations in left atrial pressure using one or more sensors carried by the cap;
transmitting data from the device to a receiver via a communication component;
receiving, by a processor, data from the one or more sensors; and
estimating left atrial pressure or changes in left atrial pressure in the heart based at least in part on the mechanical changes detected by the one or more sensors.

22 The method of claim 21, wherein detecting mechanical changes comprises measuring strain using one or more strain gauges.

23. The method of claim 21, wherein detecting mechanical changes comprises sensing mechanical deformation using one or more piezoelectric elements.

24 The method of claim 21, further comprising detecting motion or vibrations associated with cardiac activity using an accelerometer.

25. The method of claim 21, further comprising capturing heart sounds for diagnostic purposes using a microphone.

26. The method of claim 21, further comprising:
periodically calibrating the estimated left atrial pressure by cross-referencing the mechanical changes detected by the one or more sensors with real pressure data from a pressure sensor carried by the device to ensure long-term accuracy of the measurements.

27. The method of claim 21, further comprising storing historical data of left atrial pressure measurements in memory coupled to the processor.

28. The method of claim 21, further comprising analyzing trends in the estimated left atrial pressure over time and adjusting operation of the device based at least in part on the trends.

29. The method of claim 21, further comprising detecting early signs of worsening heart failure by analyzing the estimated left atrial pressure.

30. The method of claim 21, further comprising triggering an alert when the estimated left atrial pressure exceeds a predetermined threshold.

31. A method of estimating changes in left atrial pressure in a patient using an implantable cardiac device placed within the left atrial appendage of a heart, the method comprising:
detecting mechanical signals corresponding to left atrial pressure in the heart using one or more mechanical sensors carried by the device;
measuring electrocardiogram (ECG) signals of the patient using at least one electrical sensor;
receiving, by a processor, signals from the one or more mechanical sensors and the at least one electrical sensor; and
estimating changes in left atrial pressure based at least in part on comparing time delays between the ECG signals and the mechanical signals.

32. The method of claim 31, wherein detecting mechanical changes comprises detecting mechanical vibrations corresponding to left atrial pressure using one or more accelerometers.

33. The method of claim 31, wherein detecting mechanical changes comprises detecting strains corresponding to left atrial pressure using one or more strain gauges.

34. The method of claim 31, further comprising identifying components of an atrial pressure waveform of the heart, including at least one of the following: an a wave, c wave, and v wave of the heart.

35 The method of claim 34, wherein estimating the left atrial pressure comprises analyzing changes in timing and amplitude of mechanical signals.

36. The method of claim 35, further comprising determining the patient's volume status based at least in part on the changes in the timing and the amplitude of the mechanical signals.

37. The method of claim 31, further comprising delivering pacing pulses using a leadless pacemaker operatively coupled to the device.

38. The method of claim 37, wherein adjusting ae heart rate of the heart via the pacemaker to enhance identification of the patient's volume status.

39. The method of claim 31, further comprising aligning mechanical measurements with the ECG signals to correlate mechanical events with specific phases of a cardiac cycle of the heart.

40 The method of claim 31, further comprising performing waveform analysis to identify and analyze components of an atrial pressure waveform of the heart.
